SEQUENCE LISTING (1 sequence)

SEQ ID NO: 1 - protein, 191 aa, Homo sapiens [sequence omitted]

(19) United States
(12) Patent Application Publication
(10) Pub. No.: US 2009/0105134 A1
(43) Pub. Date: Apr. 23, 2009

(54) C-Terminally Pegylated Growth Hormones

(75) Inventors: Magali Zundel, Dyssegaard (DK); Bernd Peschke, Malov (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 11/815,779

(22) PCT Filed: Feb. 10, 2006
(86) PCT No.: PCT/EP2006/050822

§ 371 Date: May 5, 2008

(30) Foreign Application Priority Data
Feb. 10, 2005 (DK) PA 2005 00195

(51) Int. Cl. A61K 38/02; C07K 1/00; A61P 5/00

(52) U.S. Cl. 514/12; 530/402

(57) ABSTRACT

Conjugated growth hormones of the structure (I) are provided together with methods for manufacturing said conjugates. The conjugates are useful in therapy.

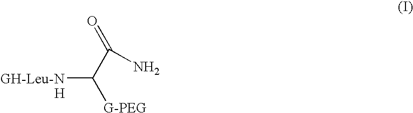

### FIELD OF THE INVENTION

The present invention relates to growth hormone compounds that have been PEGylated at the C-terminal, and to methods of preparing and using such compounds. These compounds are useful in therapy.

### BACKGROUND OF THE INVENTION

It is well-known to modify the properties and characteristics of peptides by conjugating groups to the peptide which duly changes the properties of the peptide. Such conjugation generally requires some functional group in the peptide to react with another functional group in a conjugating group. Typically, amino groups, such as the N-terminal amino group or the C-amino group in lysines, have been used in combination with a suitable acylating reagent. Alternatively, polyethylene glycol (PEG) or derivatives thereof may be attached to proteins. For a review, see Exp. Opion. Ther. Patent., 14, 859-894, 2004. It has been shown that the attachment of PEG to growth hormone may have a positive effect on the plasma half-life of growth hormone, WO 03/044056.

The use of carboxypeptidases to modify the C-terminal of peptides has been described earlier. WO 92/05271 discloses the use of carboxypeptidases and nucleophilic compounds to amidate the C-terminal carboxy group, and WO 98/38285 discloses variants of carboxypeptidase Y particular suitable for this purpose.

EP 243 929 discloses the use of carboxypeptidase to incorporate polypeptides, re-porter groups or cytotoxic agents into the C-terminal of proteins or polypeptides.

WO 2005/035553 describes methods for selective conjugation of peptides by enzymatically incorporating a functional group at the C-terminal of a peptide.

Growth hormone is a key hormone involved in the regulation of not only somatic growth, but also in the regulation of metabolism of proteins, carbohydrates and lipids. The major effect of growth hormone is to promote growth. Human growth hormone is a 191 amino acid residue protein with the sequence FPTIPLSRLFDNAMLRAHRLHQLAFDTYQEFEEAYIPKEQKYSFLQNPQTSLCFSESIPTPSN REETQQKSNLELLRISLLLIQSWLEPVQFLRSVFANSLVYGASDSNVYDLLKDLEEGIQTLMG RLEDGSPRTGQIFKQTYSKFDTNSHNDDALLKNYGLLYCFRKDMDKVETFLRIVQCRSVEG SCGF (SEQ ID NO:1).

Administration of human growth hormone and closely related variants thereof is used to treat a variety of growth hormone deficiency related diseases. Being a peptide, growth hormone is administered parenterally, i.e., by means of a needle. Growth hormone is, furthermore, characterised by a relative short half-life, hence frequent administrations are required with the corresponding pain and inconvenience for the patient. Hence, there is still a need for the provision of growth hormone compounds with improved pharmacological properties, such as e.g. prolonged half-life.

The present invention provides novel growth hormone conjugates with improved pharmacological properties as well as methods for their production.

### SUMMARY OF THE INVENTION

The present inventors have surprising found that growth hormone compounds (GH) which have a —XX-Ala sequence at their C-terminal may be PEGylated to obtain GH conjugates with improved pharmacological properties. Accordingly, in one embodiment, the present invention relates to a compound according to formula I

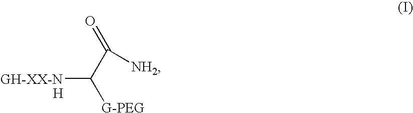

wherein GH represent a growth hormone compound; G represents a biradical of any chemical moiety; PEG represents a polyethylene glycol radical; and XX represent an amino acid residue selected from histidine, asparatic acid, arginine, phenylalanine, alanine, glycine, glutamine, glutamic acid, lysine, leucine, methionine, asparagine, serine, tyrosine, threonine, isoleucine, tryptophane, proline, and valine; and pharmaceutically acceptable salts, solvates and prodrugs thereof.

In one embodiment, the invention provides compounds according to formula I for use in therapy.

In one embodiment, the invention provides a pharmaceutical composition comprising a compound of formula I.

In one embodiment, the invention provides a therapeutic method, the method comprising the administration of a therapeutically effective amount of a compound of formula I to a patient in need thereof.

In one embodiment, the invention provides the use of a compound of formula I in the manufacture of a medicament.

In one embodiment, the invention provides a method for the manufacture of a compound of formula I, the method comprising the steps of
i) reacting in one or more steps a GH-XX-Ala with a first compound bearing one or more functional groups, which are not accessible in any of the amino acids constituting said GH-XX-Ala, in the presence of Carboxypeptidase Y (CPY) cable of catalysing the incorporation of said first compound into the C-terminal of said GH-XX-Ala to form a transacylated compound, and
ii) reacting in one or more steps said transacylated compound with a second compound comprising a PEG moiety and one or more functional groups, wherein said functional group(s) do not react with functional groups accessible in the amino acid residues constituting said GH-XX-Ala, and wherein said functional group(s) in said second compound is capable of reacting with said functional group(s) in said first compound so that a covalent bond between said transacylated compound and said second compound is formed.

It is a further objective of the present invention to provide growth hormone compounds which have been extended at the C-terminal by a —XX-Ala sequence, i.e. compound of the formula GH-XX-Ala.

It is a still further objective of the present invention to provide a method for improving the properties of a GH by conjugation said peptide according to the methods of the pre-sent invention.

### DESCRIPTION OF THE FIGURES

FIG. 1: Vector map of pNNC13.4 encoding Zbasic2mt-D4K-hGH-Leu-Ala. Sac II and BamHI sites used for insertion of the PCR amplicon can be seen.

### DEFINITIONS

In the present context, the term “transacylation” is intended to indicate a reaction in which a leaving group is exchanged for a nucleophile, wherein a nucleophile is understood to be an electron-rich reagent that tends to attack the nucleus of carbons. Transpeptidation is one example of a transacylation.

In the present context, the term “not accessible” is intended to indicate that some-thing is absent or de facto absent in the sense that it cannot be reached. When it is stated that functional groups are not accessible in a peptide to be conjugated it is intended to indicate that said functional group is absent from the peptide or, if present, in some way pre-vented from taking part in reactions. By way of example, said functional group could be buried deep in the structure of the peptide so that it is shielded from participating in the reaction. It is recognised that whether or not a functional group is accessible depends on the reaction conditions. It may be envisaged that, e.g. in the presence of denaturing agents or at elevated temperatures the peptide may unfold to expose otherwise not accessible functional groups. It is to be understood that “not accessible” means “not accessible at the reaction condition chosen for the particular reaction of interest”.

In the present context, the term “oxime bond” is intended to indicate a moiety of the formula-C═N—O—.

In the present context, the term “hydrazone bond” is intended to indicate a moiety of the formula —C═N—N—.

In the present context, the term “phenylhydrazone bond” is intended to indicate a moiety of the formula

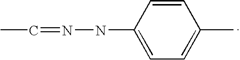

In the present context, the term “semicarbazone bond” is intended to indicate a moiety of the formula —C═N—N—C(O)—N—.

The term “alkane” is intended to indicate a saturated, linear, branched and/or cyclic hydrocarbon. Unless specified with another number of carbon atoms, the term is intended to indicate hydrocarbons with from 1 to 30 (both included) carbon atoms, such as 1 to 20 (both included), such as from 1 to 10 (both included), e.g. from 1 to 5 (both included). The terms alkyl and alkylene refer to the corresponding radical and bi-radical, respectively.

The term “alkene” is intended to indicate linear, branched and/or cyclic hydrocarbons comprising at least one carbon-carbon double bond. Unless specified with another number of carbon atoms, the term is intended to indicate hydrocarbons with from 2 to 30 (both included) carbon atoms, such as 2 to 20 (both included), such as from 2 to 10 (both included), e.g. from 2 to 5 (both included). The terms alkenyl and alkenylene refer to the corresponding radical and bi-radical, respectively.

The term “alkyne” is intended to indicate linear, branched and/or cyclic hydrocarbons comprising at least one carbon-carbon triple bond, and it may optionally comprise one or more carbon-carbon double bonds. Unless specified with another number of carbon atoms, the term is intended to indicate hydrocarbons with from 2 to 30 (both included) carbon atoms, such as from 2 to 20 (both included), such as from 2 to 10 (both included), e.g. from 2 to 5 (both included). The terms alkynyl and alkynylene refer to the corresponding radical and bi-radical, respectively.

The term “homocyclic aromatic compound” is intended to indicate aromatic hydrocarbons, such as benzene and naphthalene.

The term “heterocyclic compound” is intended to indicate a cyclic compound comprising 5, 6 or 7 ring atoms from which 1, 2, 3 or 4 are hetero atoms selected from N, O and/or S. Examples include heterocyclic aromatic compounds, such as thiophene, furan, pyran, pyrrole, imidazole, pyrazole, isothiazole, isooxazole, pyridine, pyrazine, pyrimidine, pyridazine, as well as their partly or fully hydrogenated equivalents, such as piperidine, pirazolidine, pyrrolidine, pyrroline, imidazolidine, imidazoline, piperazine and morpholine.

The terms “hetero alkane”, “hetero alkene” and “hetero alkyne” is intended to indicate alkanes, alkenes and alkynes as defined above, in which one or more hetero atom or group have been inserted into the structure of said moieties. Examples of hetero groups and atoms include —O—, —S—, —S(O)—, —S(O)₂—, —C(O)— —C(S)— and —N(R*)—, wherein R* represents hydroqen or C₁-C₆-alkyl. Examples of heteroalkanes include.

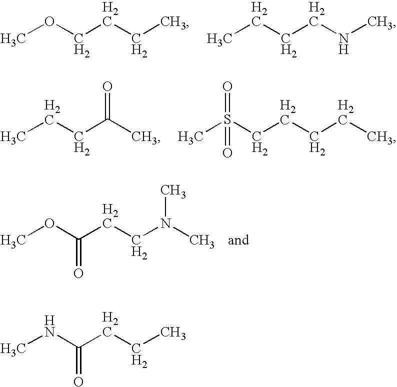

The term “radical” or “biradical” is intended to indicate a compound from which one or two, respectively, hydrogen atoms have been removed. When specifically stated, a radical may also indicate the moiety formed by the formal removal of a larger group of atoms, e.g. hydroxyl, from a compound.

The term “halogen” is intended to indicate members of the seventh main group of the periodic table, e.g. F, Cl, Br and I.

The term “PEG” is intended to indicate polyethylene glycol of a molecular weight between approximately 100 and approximately 1,000,000 Da, including analogues thereof, wherein for instance the terminal OH-group has been replaced by an alkoxy group, such as e.g. a methoxy group, an ethoxy group or a propoxy group. In particular, the PEG wherein the terminal —OH group has been replaced by methoxy is referred to as mPEG.

The term “mPEG” (or more properly “mPEGyl”) means a polydisperse or monodisperse radical of the structure

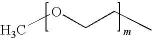

wherein m is an integer larger than 1. Thus, a mPEG wherein m is 90 has a molecular weight of 3975 Da, i.e. approx 4 kDa. Likewise, a mPEG with an average molecular weight of 20 kDa has an average m of 454. Due to the process for producing mPEG these molecules often have a distribution of molecular weights. This distribution is described by the polydispersity index.

The term “polydispersity index” as used herein means the ratio between the weight average molecular weight and the number average molecular weight, as known in the art of polymer chemistry (see e.g. “Polymer Synthesis and Characterization”, J. A. Nairn, University of Utah, 2003). The polydispersity index is a number which is greater than or equal to one, and it may be estimated from Gel Permeation Chromatographic data. When the polydispersity index is 1, the product is monodisperse and is thus made up of compounds with a single molecular weight. When the polydispersity index is greater than 1 it is a measure of the polydispersity of that polymer, i.e. how broad the distribution of polymers with different molecular weights is.

The use of for example “mPEG20000” in formulas, compound names or in molecular structures indicates an mPEG residue wherein mPEG is polydisperse and has a molecular weight of approximately 20 kDa.

The polydispersity index typically increases with the molecular weight of the PEG or mPEG. When reference is made to 5 kDa PEG and in particular 5 kDa mPEG it is intended to indicate a compound (or in fact a mixture of compounds) with a polydisperisty index below 1.06, such as below 1.05, such as below 1.04, such as below 1.03, such as between 1.02 and 1.03. When reference is made to 10 kDa PEG and in particular 10 kDa mPEG it is intended to indicate a compound (or in fact a mixture of compounds) with a polydisperisty index below 1.06, such as below 1.05, such as below 1.04, such as below 1.03, such as between 1.02 and 1.03. When reference is made to 15 kDa PEG and in particular 15 kDa mPEG it is intended to indicate a compound (or in fact a mixture of compounds) with a polydisperisty index below 1.06, such as below 1.05, such as below 1.04, such as below 1.03, such as between 1.02 and 1.03. When reference is made to 20 kDa PEG and in particular 20 kDa mPEG it is intended to indicate a compound (or in fact a mixture of compounds) with a polydisperisty index below 1.06, such as below 1.05, such as below 1.04, such as below 1.03, such as between 1.02 and 1.03. When reference is made to 30 kDa PEG and in particular 30 kDa mPEG it is intended to indicate a compound (or in fact a mixture of compounds) with a polydisperisty index below 1.06, such as below 1.05, such as below 1.04, such as below 1.03, such as between 1.02 and 1.03. When reference is made to 40 kDa PEG and in particular 40 kDa mPEG it is intended to indicate a compound (or in fact a mixture of compounds) with a polydisperisty index below 1.06, such as below 1.05, such as below 1.04, such as below 1.03, such as between 1.02 and 1.03. When reference is made to 60 kDa PEG and in particular 60 kDa mPEG it is intended to indicate a compound (or in fact a mixture of compounds) with a polydisperisty index below 1.06, such as below 1.05, such as below 1.04, such as below 1.03, such as between 1.02 and 1.03.

In the present context, the words “peptide” and “protein” are used interchangeably and are intended to indicate the same. The term “peptide” is intended to indicate a compound with two or more amino acid residues linked by a peptide bond. The amino acids may be natural or unnatural. The term is also intended to include said compounds substituted with other peptides, saccharides, lipids, or other organic compound, as well as compounds wherein one or more amino acid residue have been chemically modified and peptides comprising a prosthetic group.

In the present context, the term “aryl” is intended to indicate a carbocyclic aromatic ring radical or a fused aromatic ring system radical wherein at least one of the rings are aromatic. Typical aryl groups include phenyl, biphenylyl, naphthyl, and the like.

The term “heteroaryl”, as used herein, alone or in combination, refers to an aromatic ring radical with for instance 5 to 7 member atoms, or to a fused aromatic ring system radical with for instance from 7 to 18 member atoms, wherein at least one ring is aromatic, containing one or more heteroatoms as ring atoms selected from nitrogen, oxygen, or sulfur heteroatoms, wherein N-oxides and sulfur monoxides and sulfur dioxides are permissible heteroaromatic substitutions. Examples include furanyl, thienyl, thiophenyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, quinolinyl, isoquinolinyl, benzofuranyl, benzothiophenyl, indolyl, and indazolyl, and the like.

The term “conjugate” as a noun is intended to indicate a modified peptide, i.e. a peptide with a moiety bonded to it to modify the properties of said peptide. As a verb, the term is intended to indicate the process of bonding a moiety to a peptide to modify the properties of said peptide.

As used herein, the term “prodrug” indicates biohydrolyzable amides and biohydrolyzable esters and also encompasses a) compounds in which the biohydrolyzable functionality in such a prodrug is encompassed in the compound according to the present invention, and b) compounds which may be oxidized or reduced biologically at a given functional group to yield drug substances according to the present invention. Examples of these functional groups include 1,4-dihydropyridine, N-alkylcarbonyl-1,4-dihydropyridine, 1,4-cyclohexadiene, tertbutyl, and the like.

As used herein, the term “biohydrolyzable ester” is an ester of a drug substance (in casu, a compound according to the invention) which either a) does not interfere with the biological activity of the parent substance but confers on that substance advantageous properties in vivo such as duration of action, onset of action, and the like, or b) is biologically inactive but is readily converted in vivo by the subject to the biologically active principle. The advantage is, for example increased solubility or that the biohydrolyzable ester is orally absorbed from the gut and is transformed to a compound according to the present invention in plasma. Many examples of such are known in the art and include by way of example lower alkyl esters (e.g., C₁-C₄), lower acyloxyalkyl esters, lower alkoxyacyloxyalkyl esters, alkoxyacyloxy esters, alkyl acylamino alkyl esters, and choline esters.

As used herein, the term “biohydrolyzable amide” is an amide of a drug substance (in casu, a compound according to the present invention) which either a) does not interfere with the biological activity of the parent substance but confers on that substance advantageous properties in vivo such as duration of action, onset of action, and the like, or b) is biologically inactive but is readily converted in vivo by the subject to the biologically active principle. The advantage is, for example increased solubility or that the biohydrolyzable amide is orally absorbed from the gut and is transformed to a compound according to the present invention in plasma. Many examples of such are known in the art and include by way of example lower alkyl amides, α-amino acid amides, alkoxyacyl amides, and alkylaminoalkylcarbonyl amides.

In the present context, the term “pharmaceutically acceptable salt” is intended to indicate salts which are not harmful to the patient. Such salts include pharmaceutically acceptable acid addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids and the like. Further examples of pharmaceutically acceptable inorganic or organic acid addition salts include the pharmaceutically acceptable salts listed in J. Pharm. Sci. 1977, 66, 2, which is incorporated herein by reference. Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like.

A “therapeutically effective amount” of a compound as used herein means an amount sufficient to cure, alleviate or partially arrest the clinical manifestations of a given disease and its complications. An amount adequate to accomplish this is defined as “therapeutically effective amount”. Effective amounts for each purpose will depend on the severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician or veterinary.

The term “treatment” and “treating” as used herein means the management and care of a patient for the purpose of combating a condition, such as a disease or a disorder. The term is intended to include the full spectrum of treatments for a given condition from which the patient is suffering, such as administration of the active compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of a patient for the purpose of combating the disease, condition, or disorder and includes the administration of the active compounds to prevent the onset of the symptoms or complications. The patient to be treated is preferably a mammal, in particular a human being, but it may also include animals, such as dogs, cats, cows, sheep and pigs.

### DESCRIPTION OF THE INVENTION

In one embodiment of the invention, XX represents Leu, i.e. relates to a compound according to formula I with a structure of formula Ia

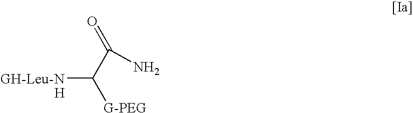

In one embodiment, the invention relates to a compound according to formula Ia with the structure of formula Ib

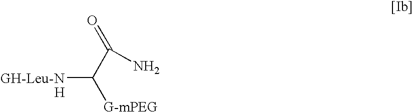

wherein GH represents a growth hormone compound; mPEG represents any straight or branched methoxy polyethylene glycol moiety with a molecular weight between 0.1 kDa and 1000 kDa; and
G represents R-A-E; wherein
R and E both independently represent a bond or a linker, and A represents a biradical; and pharmaceutically acceptable salts, solvates and prodrugs thereof.

In a particular embodiment, the compound of formula Ib has a structure according to formula Ic

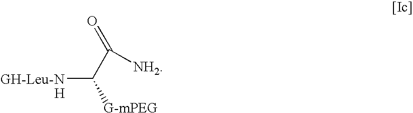

In one embodiment, the invention relates to a compound according to formula Ia with the structure of formula Id, Ie, or If,

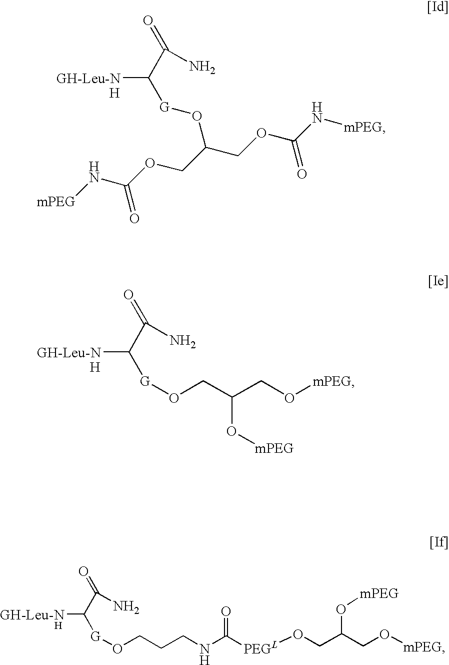

wherein GH represents a growth hormone compound;
mPEG represents any straight or branched methoxy polyethylene glycol moiety with a molecular weight between 0.1 kDa and 1000 kDa; PEG^{L} is a di-radical of a polyethylenglycol-moiety with a molecular weight between 2 kDa and 5 kDa, and
G represents R-A-E; wherein
R and E both independently represent a bond or a linker, and A represents a biradical; and pharmaceutically acceptable salts, solvates and prodrugs thereof.

In a particular embodiment, the compounds of formulas Id, Ie, and If have the structures according to formulas Ig, Ih, and Ii, respectively,

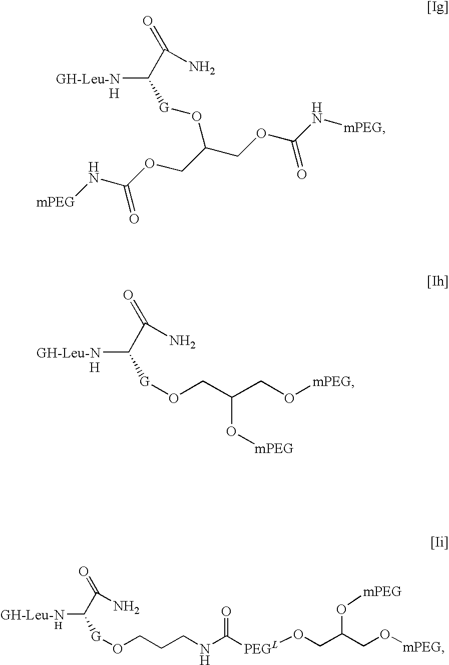

wherein PEG^{L} is a di-radical of a polyethylenglycol-moiety with a molecular weight between 2 kDa and 5 kDa.

In one embodiment, GH represents human growth hormone, hGH.

In one embodiment, the GH is a derivative of hGH, obtained by chemically modifying one or more amino acids in the hGH sequence. Exemplary GH derivatives include, but are not limited to, hGH covalently conjugated to one or more other moities.

In one embodiment, GH is a variant of hGH, wherein a variant is understood to be the compound obtained by substituting one or more amino acid residues in the hGH sequence with another natural or unnatural amino acid; and/or by adding one or more natural or unnatural amino acids to the hGH sequence; and/or by deleting one or more amino acid residue from the hGH sequence, wherein any of these steps may optionally be followed by further derivatization of one or more amino acid residues. In particular, such substitutions are conservative in the sense that one amino acid residue is substituted by another amino acid residue from the same group, i.e. by another amino acid residue with similar properties. Amino acids may conveniently be divided in the following groups based on their properties: Basic amino acids (such as arginine, lysine, histidine), acidic amino acids (such as glutamic acid and aspartic acid), polar amino acids (such as glutamine, cysteine and asparagine), hydrophobic amino acids (such as leucine, isoleucine, proline, methionine and valine), aromatic amino acids (such as phenylalanine, tryptophan, tyrosine) and small amino acids (such as glycine, alanine, serine and threonine.).

In one embodiment, GH has at least 80%, such as at least 85%, such as at least 90%, such as at least 95% identity with hGH. In one embodiment, said identities to hGH is coupled to at least 20%, such as at least 40%, such as at least 60%, such as at least 80% of the growth hormone activity of hGH as determined in assay I herein.

The term “identity” as known in the art, refers to a relationship between the sequences of two or more proteins, as determined by comparing the sequences. In the art, “identity” also means the degree of sequence relatedness between proteins, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related proteins can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math., 48:1073 (1988).

Preferred methods to determine identity are designed to give the largest match between the sequences tested. Methods to determine identity are described in publicly available computer programs. Preferred computer program methods to determine identity between two sequences include the GCG program package, including GAP (Devereux et al., Nucl. Acid. Res., 12:387 (1984); Genetics Computer Group, University of Wisconsin, Madison, Wis.), BLASTP, BLASTN, and FASTA (Altschul et al., J. Mol. Biol., 215:403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well known Smith Waterman algorithm may also be used to determine identity.

For example, using the computer algorithm GAP (Genetics Computer Group, University of Wisconsin, Madison, Wis.), two proteins for which the percent sequence identity is to be determined are aligned for optimal matching of their respective amino acids (the “matched span”, as determined by the algorithm). A gap opening penalty (which is calculated as 3.times. the average diagonal; the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. A standard comparison matrix (see Dayhoff et al., Atlas of Protein Sequence and Structure, vol. 5, supp. 3 (1978) for the PAM 250 comparison matrix; Henikoff et al., Proc. Natl. Acad. Sci. USA, 89:10915-10919 (1992) for the BLOSUM 62 comparison matrix) is also used by the algorithm.

Preferred parameters for a protein sequence comparison include the following:

Algorithm: Needleman et al., J. Mol. Biol, 48:443-453 (1970); Comparison matrix: BLOSUM 62 from Henikoff et al., Proc. Natl. Acad. Sci. USA, 89:10915-10919 (1992); Gap Penalty: 12, Gap Length Penalty: 4, Threshold of Similarity: 0.

The GAP program is useful with the above parameters. The aforementioned parameters are the default parameters for protein comparisons (along with no penalty for end gaps) using the GAP algorithm.

Both R and E independently represent a bond or a linker. These linkers may be absent (i.e. R and/or E represents a bond) or selected from amongst alkane, alkene or alkyne diradicals and hetero alkane, hetero alkene and hetero alkyne diradicals, wherein one or more optionally substituted aromatic homocyclic biradical or biradical of a heterocyclic compound, e.g. phenylene or piperidine biradical may be inserted into the aforementioned biradicals. It is to be understood that said linkers may also comprise substitutions by groups selected from amongst hydroxyl, halogen, nitro, cyano, carboxyl, aryl, alkyl and heteroaryl.

Typical examples of E and R include bi-radicals of straight, branched and/or cyclic C₁₋₁₀alkane, C₂₋₁₀alkene, C₂₋₁₀alkyne, C₁₋₁₀heteroalkane, C₂₋₁₀heteroalkene, C₂₋₁₀heteroalkyne, wherein one or more homocyclic aromatic compound biradical or heterocyclic compound biradical may be inserted. Particular examples of E and R include

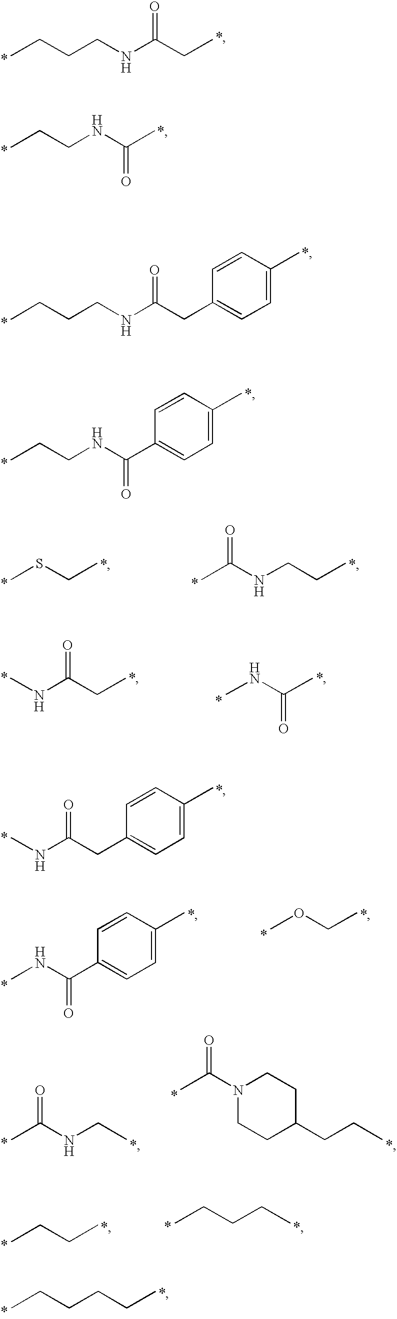

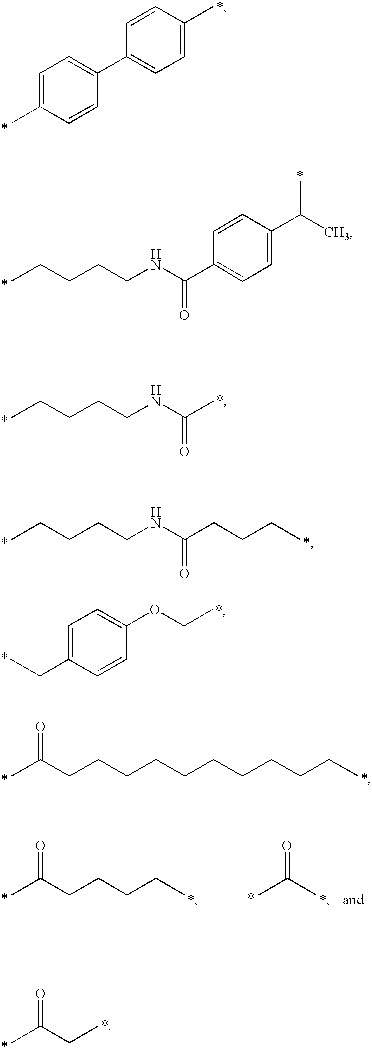

A represents the biradical formed in the reaction between X and Y as described below. Examples of A include oxime bond, hydrazone bond, phenylhydrazone bond, semicarbazone moiety, triazole bond, isooxazolidine bond, amide bond or aralkyne bond.

In one embodiment, G, i.e. R-A-E, represents

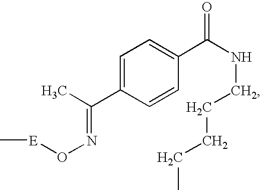

such as

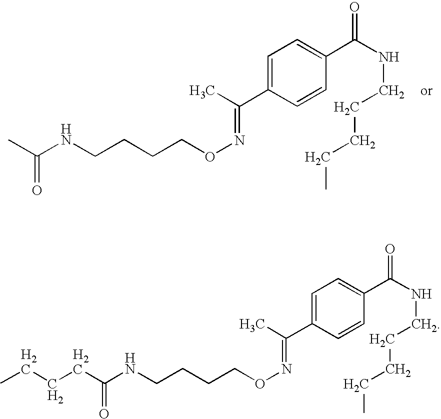

In one embodiment, G, i.e. R-A-E represents

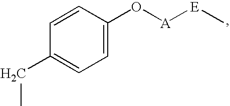

such as, e.g.,

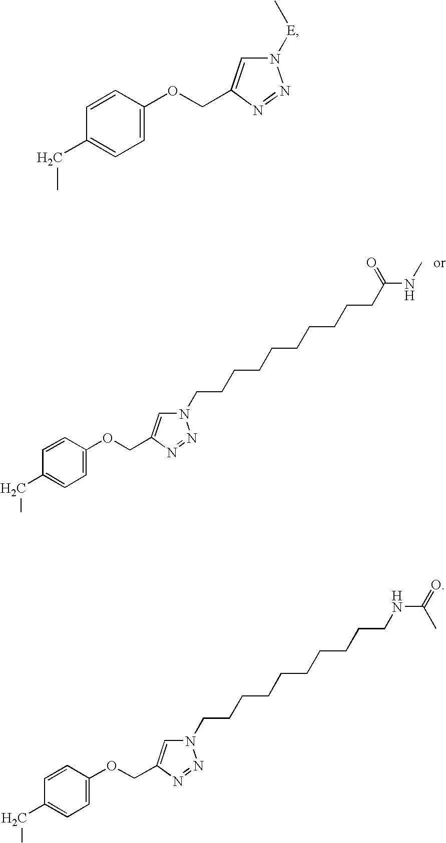

In one embodiment, G, i.e., R-A-E, represents

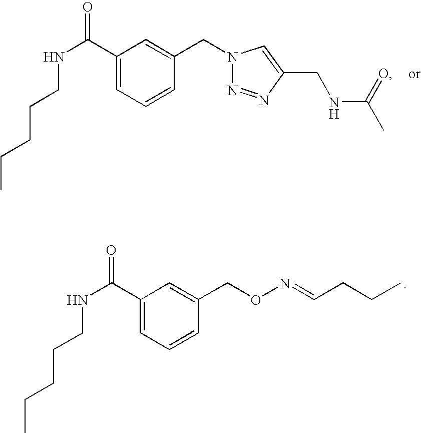

In one embodiment, G, i.e., R-A-E, represents

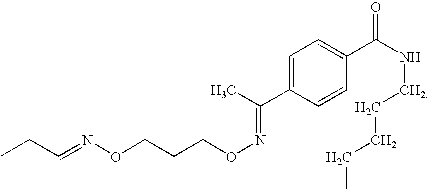

In one embodiment, mPEG represents a mPEG with a molecular weight between around 0.5 kDa and around 100 kDa, such as between around 5 kDa and around 80 kDa, such as between around 10 kDa and around 60 kDa, such as between around 10 kDa and around 40 kDa. In particular mPEG with a molecular weight between around 10 kDa and around 30 kDa, such as between around 10 kDa and around 20 kDa. Particular mentioning is made of mPEG with a molecular weight around 5 kDa, around 10 kDa, around 15 kDa, around 20 kDa, around 30 kDa, around 40 kDa, and around 60 kDa. Said mPEG may be branched in the sense that the moiety comprises more than one mPEG arm, typically two or three arms. As an example, mPEG with a molecular weight around 40 kDa may be difficult to prepare as a single chain molecule, but may be prepared as a branched mPEG comprising two mPEG arms, each with a molecular weight of around 20 kDa.

Particular examples of compounds of formula I include

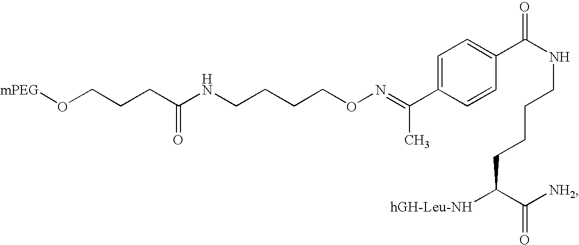

in which mPEG has a molecular weight around 5 kDa, 10 kDa, 15 kDa, 20 kDa, 30 kDa, 40 kDa or 60 kDa;

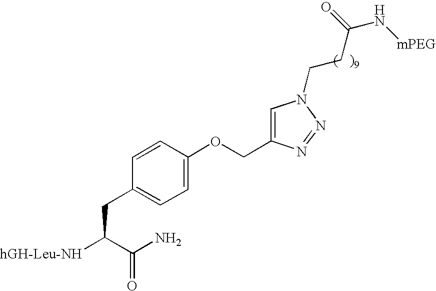

in which mPEG has a molecular weight around 5 kDa, 10 kDa, 15 kDa, 20 kDa, 30 kDa, 40 kDa or 60 kDa;

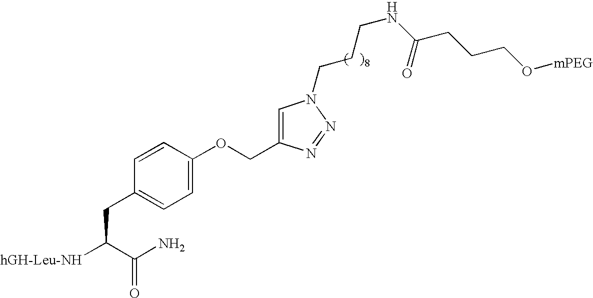

in which mPEG has a molecular weight around 5 kDa, 10 kDa, 15 kDa, 20 kDa, 30 kDa, 40 kDa or 60 kDa;

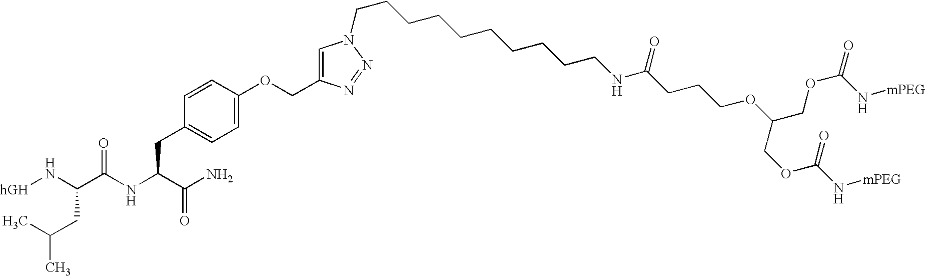

in which each mPEG has a molecular weight of about 5 kDa, 10 kDa, 15 kDa, 20 kDa, 30 kDa, 40 kDa or 60 kDa;

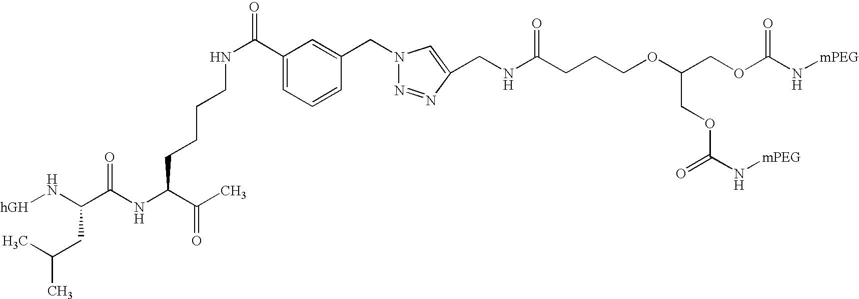

in which each mPEG has a molecular weight of about 5 kDa, 10 kDa, 15 kDa, 20 kDa, 30 kDa, 40 kDa or 60 kDa;

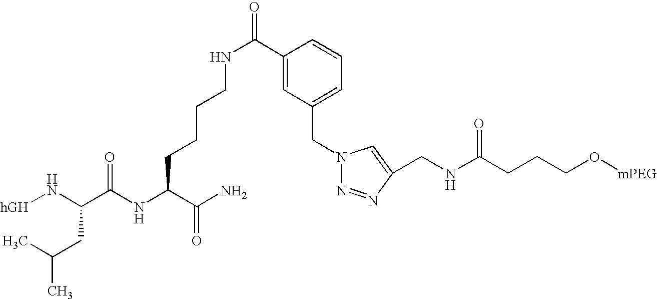

in which mPEG has a molecular weight of about 5 kDa, 10 kDa, 15 kDa, 20 kDa, 30 kDa, 40 kDa or 60 kDa;

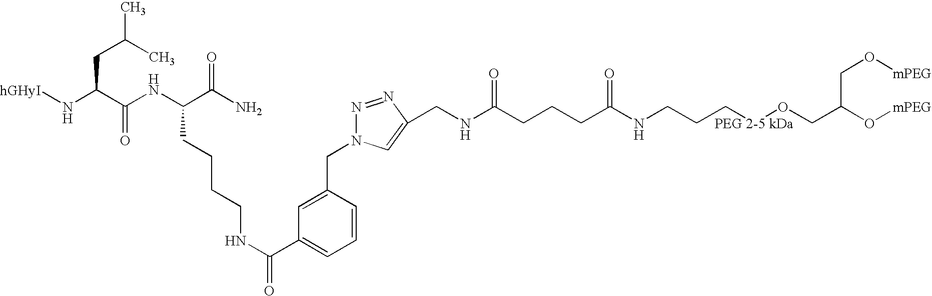

in which each mPEG has a molecular weight of about 5 kDa, 10 kDa, 15 kDa, 20 kDa, 30 kDa, 40 kDa or 60 kDa;

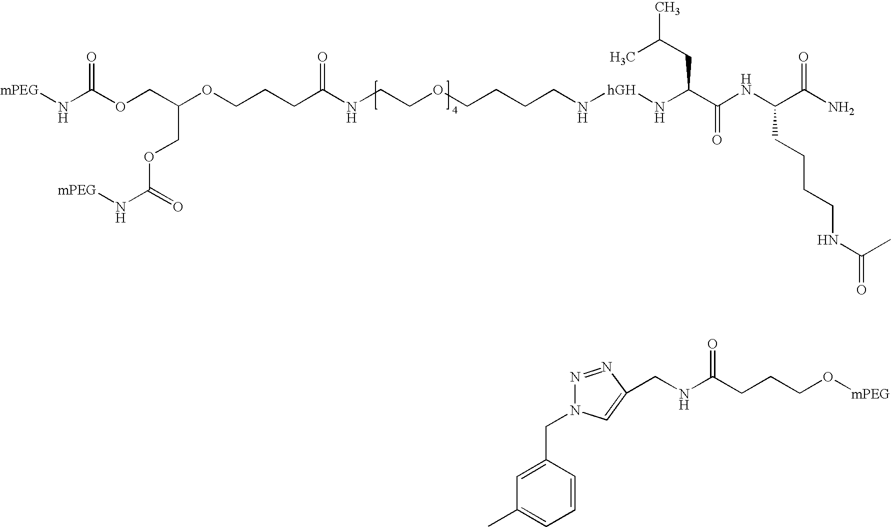

in which each mPEG has a molecular weight of about 5 kDa, 10 kDa, 15 kDa, 20 kDa, 30 kDa, 40 kDa or 60 kDa;

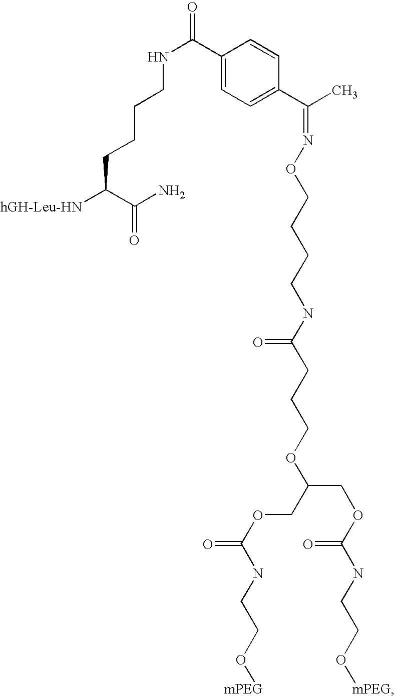

in which each mPEG has a molecular weight of about 5 kDa, 10 kDa, 15 kDa, 20 kDa, 30 kDa, 40 kDa or 60 kDa;

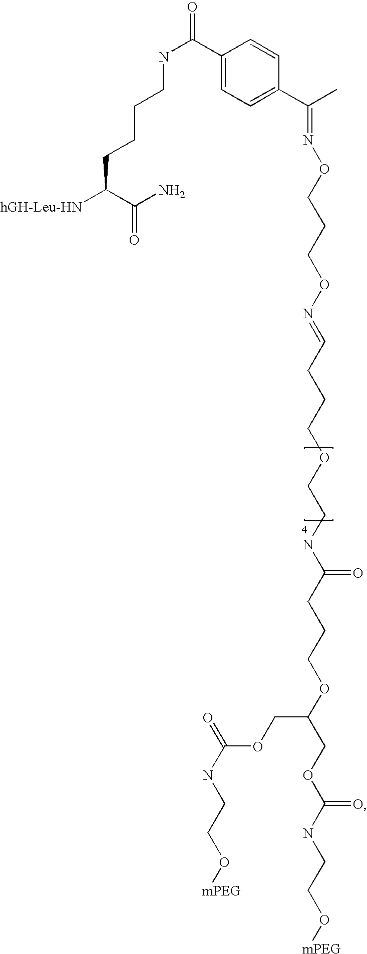

in which mPEG has a molecular weight of about 5 kDa, 10 kDa, 15 kDa, 20 kDa, 30 kDa, 40 kDa or 60 kDa;

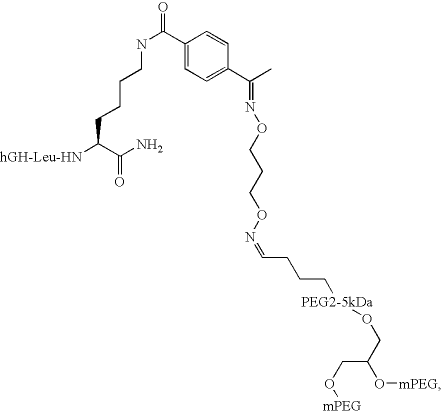

in which mPEG has a molecular weight of about 5 kDa, 10 kDa, 15 kDa, 20 kDa, 30 kDa, 40 kDa or 60 kDa; and

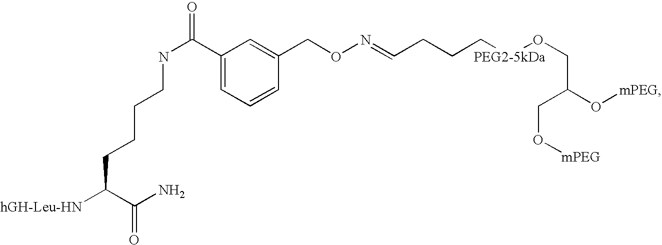

in which mPEG has a molecular weight of about 5 kDa, 10 kDa, 15 kDa, 20 kDa, 30 kDa, 40 kDa or 60 kDa.

The compounds of the present invention have improved pharmacological properties compared to the corresponding un-conjugated growth hormone, which is also referred to as the parent compound. In this context, the unconjugated GH may both represent GH and GH-XX-Ala. Examples of such pharmacological properties include functional in vivo half-life, immunogencity, renal filtration protease protection and albumin binding.

The term “functional in vivo half-life” is used in its normal meaning, i.e., the time at which 50% of the biological activity of the GH or GH conjugate are still present in the body/target organ, or the time at which the activity of the GH or GH conjugate is 50% of its initial value. As an alternative to determining functional in vivo half-life, “in vivo plasma half-life” may be determined, i.e., the time at which 50% of the GH or GH conjugate circulate in the plasma or bloodstream prior to being cleared. Determination of plasma half-life is often more simple than determining functional half-life and the magnitude of plasma half-life is usually a good indication of the magnitude of functional in vivo half-life. Alternative terms to plasma half-life include serum half-life, circulating half-life, circulatory half-life, serum clearance, plasma clearance, and clearance half-life.

The term “increased” as used in connection with the functional in vivo half-life or plasma half-life is used to indicate that the relevant half-life of the GH conjugate is statistically significantly increased relative to that of the parent GH, as determined under comparable conditions. For instance the relevant half-life may be increased by at least about 25%, such as by at lest about 50%, e.g., by at least about 100%, 150%, 200%, 250%, or 500%. In one embodiment, the compounds of the present invention exhibit an increase in half-life of at least about 5 h, preferably at least about 24 h, more preferably at least about 72 h, and most preferably at least about 7 days, relative to the half-life of the parent GH.

Measurement of in vivo plasma half-life can be carried out in a number of ways as described in the literature. An increase in in vivo plasma half-life may be quantified as a decrease in clearance (CL) or as an increase in mean residence time (MRT). Conjugated GH of the present invention for which the CL is decreased to less than 70%, such as less than 50%, such than less than 20%, such than less than 10% of the CL of the parent GH as determined in a suitable assay is said to have an increased in vivo plasma half-life. Conjugated GH of the present invention for which MRT is increased to more than 130%, such as more than 150%, such as more than 200%, such as more than 500% of the MRT of the parent GH in a suitable assay is said to have an increased in vivo plasma half-life. Clearance and mean residence time can be assessed in standard pharmacokinetic studies using suitable test animals. It is within the capabilities of a person skilled in the art to choose a suitable test animal for a given protein. Tests in human, of course, represent the ultimate test. Suitable text animals include normal, Sprague-Dawley male rats, mice and cynomolgus monkeys. Typically the mice and rats are in injected in a single subcutaneous bolus, while monkeys may be injected in a single subcutaneous bolus or in a single iv dose. The amount injected depends on the test animal. Subsequently, blood samples are taken over a period of one to five days as appropriate for the assessment of CL and MRT. The blood samples are conveniently analysed by ELISA techniques.

The term “Immunogenicity” of a compound refers to the ability of the compound, when administered to a human, to elicit a deleterious immune response, whether humoral, cellular, or both. In any human sub-population, there may exist individuals who exhibit sensitivity to particular administered proteins. Immunogenicity may be measured by quantifying the presence of growth hormone antibodies and/or growth hormone responsive T-cells in a sensitive individual, using conventional methods known in the art. In one embodiment, the conjugated GH of the present invention exhibit a decrease in immunogenicity in a sensitive individual of at least about 10%, preferably at least about 25%, more preferably at least about 40% and most preferably at least about 50%, relative to the immunogenicity for that individual of the parent GH.

The term “protease protection” or “protease protected” as used herein is intended to indicate that the conjugated GH of the present invention is more resistant to the plasma peptidase or proteases than is the parent GH. Protease and peptidase enzymes present in plasma are known to be involved in the degradation of circulating proteins, such as e.g. circulating peptide hormones, such as growth hormone.

Growth hormone may be susceptible to degradation by for instance thrombin, plasmin, subtilisin, and chymotrypsin-like serine proteinase. Assays for determination of degradation of these proteases are described ain J. Biotech., 65, 183, 1998. In one embodiment, the rate of hydrolysis of the GH conjugate is less than 70%, such as less than 40%, such as less than 10% of that of the parent GH.

The most abundant protein component in circulating blood of mammalian species is serum albumin, which is normally present at a concentration of approximately 3 to 4.5 grams per 100 milliters of whole blood. Serum albumin is a blood protein of approximately 70,000 daltons which has several important functions in the circulatory system. It functions as a transporter of a variety of organic molecules found in the blood, as the main transporter of various metabolites such as fatty acids and bilirubin through the blood, and, owing to its abundance, as an osmotic regulator of the circulating blood. Serum albumin has a half-life of more than one week, and one approach to increasing the plasma half-life of proteins has been to conjugate to the protein a group that binds to serum albumin. Albumin binding property may be determined as described in J. Med. Chem., 43, 2000, 1986-1992, which is incorporated herein by reference.

Compounds of formula (I) exert growth hormone activity and may as such be used in the treatment of diseases or states which will benefit from an increase in the amount of circulating growth hormone. In particular, the invention provides a method for the treatment of growth hormone deficiency (GHD); Turner Syndrome; Prader-Willi syndrome (PWS); Noonan syndrome; Down syndrome; chronic renal disease, juvenile rheumatoid arthritis; cystic fibrosis, HIV-infection in children receiving HAART treatment (HIV/HALS children); short children born short for gestational age (SGA); short stature in children born with very low birth weight (VLBW) but SGA; skeletal dysplasia; hypochondroplasia; achondroplasia; idiopathic short stature (ISS); GHD in adults; fractures in or of long bones, such as tibia, fibula, femur, humerus, radius, ulna, clavicula, matacarpea, matatarsea, and digit; fractures in or of spongious bones, such as the scull, base of hand, and base of food; patients after tendon or ligament surgery in e.g. hand, knee, or shoulder; patients having or going through distraction oteogenesis; patients after hip or discus replacement, meniscus repair, spinal fusions or prosthesis fixation, such as in the knee, hip, shoulder, elbow, wrist or jaw; patients into which osteosynthesis material, such as nails, screws and plates, have been fixed; patients with non-union or mal-union of fractures; patients after osteatomia, e.g. from tibia or 1^{st} toe; patients after graft implantation; articular cartilage degeneration in knee caused by trauma or arthritis; osteoporosis in patients with Turner syndrome; osteoporosis in men; adult patients in chronic dialysis (APCD); malnutritional associated cardiovascular disease in APCD; reversal of cachexia in APCD; cancer in APCD; chronic abstractive pulmonal disease in APCD; HIV in APCD; elderly with APCD; chronic liver disease in APCD, fatigue syndrome in APCD; Crohn's disease; impaired liver function; males with HIV infections; short bowel syndrome; central obesity; HIV-associated lipodystrophy syndrome (HALS); male infertility; patients after major elective surgery, alcohol/drug detoxification or neurological trauma; aging; frail elderly; osteo-arthritis; traumatically damaged cartilage; erectile dysfunction; fibromyalgia; memory disorders; depression; traumatic brain injury; subarachnoid haemorrhage; very low birth weight; metabolic syndrome; glucocorticoid myopathy; or short stature due to glucucorticoid treatment in children, the method comprising administering to a patient in need thereof a therapeutically effective amount of a compound according to formula (I)

In one aspect, the invention provides a method for the acceleration of the healing of muscle tissue, nervous tissue or wounds; the acceleration or improvement of blood flow to damaged tissue; or the decrease of infection rate in damaged tissue, the method comprising administration to a patient in need thereof an effective amount of a therapeutically effective amount of a compound of formula I.

In one embodiment, the invention relates to the use of compounds according to formula I in the manufacture of diseases benefiting from an increase in the growth hormone plasma level, such as the disease mentioned above.

A typical parenteral dose is in the range of 10⁻⁹ mg/kg to about 100 mg/kg body weight per administration. Typical administration doses are from about 0.0000001 to about 10 mg/kg body weight per administration. The exact dose will depend on e.g. indication, medicament, frequency and mode of administration, the sex, age and general condition of the subject to be treated, the nature and the severity of the disease or condition to be treated, the desired effect of the treatment and other factors evident to the person skilled in the art.

Typical dosing frequencies are twice daily, once daily, bi-daily, twice weekly, once weekly or with even longer dosing intervals. Due to the prolonged half-lifes of the fusion proteins of the present invention, a dosing regime with long dosing intervals, such as twice weekly, once weekly or with even longer dosing intervals is a particular embodiment of the invention.

Many diseases are treated using more than one medicament in the treatment, either concomitantly administered or sequentially administered. It is therefore within the scope of the present invention to use compounds of formula (I) in therapeutic methods for the treatment of one of the above mentioned diseases in combination with one or more other therapeutically active compound normally used in the treatment said diseases. By analogy, it is also within the scope of the present invention to use compounds of formula (I) in combination with other therapeutically active compounds normally used in the treatment of one of the above mentioned diseases in the manufacture of a medicament for said disease.

Compounds of formula I may advantageously be prepared in a two-step enzyme catalysed reaction. The present inventors have surprisingly found that Carboxypeptidase Y (CPY) is particularly well-suited to incorporate into the C-terminal of growth hormones compounds (GH) which have been extended at the C-terminal by a —XX-Ala sequence, a first compound comprising one or more functional groups, which are not accessible in the GH, to form a transacylated compound, and that this transacylated compound may subsequently be reacted with another compound comprising a PEG moiety and one or more functional groups which react with the functional group of the first compound but not with other functional groups accessible in the GH. Such method provides a high degree of specificity in that CPY only catalyses the incorporation at the C-terminal, and the two functional groups are selected so that they only react with each other, not with other functional groups accessible in the GH. In this way, the PEG moiety is only attached at the C-terminal, and by selecting the functional groups, the number of PEG moieties can be controlled.

From the above definition of growth hormone compound it is clear that the above method may also be used on GH which themselves have a C-terminal —XX-Ala sequence. Alternatively, the above method includes an initial step in which the —XX-Ala sequence is attached to the C-terminal of GH. This may be done using standard protein chemistry techniques, e.g. de novo synthesis. Alternatively, GH-XX-Ala may be produced using standard genetic engineering techniques in which a nucleic acid sequence encoding GH-XX-Ala is inserted in a suitable vector, said vector is introduced into a suitable host cell which is fermented to allow the isolation of GH-XX-Ala from the fermentation broth, e.g. after lysis of the cells.

Carboxypeptidease Y belongs to the classification groups E.C. 3.4.16.5. The in vivo reaction catalysed by said enzyme is the hydrolysis of the C-terminal amino acid residue. During the catalytic cycle an enzyme-substrate complex is formed which under normal in vivo conditions is subjected to a nucleophilic attack by a water molecule, which eventually leads to the hydrolysis of the peptide bond. In the methods of the present invention, however, a nucleophilic reagent is added, which can out compete water as a nucleophile. Moreover, the water activity may be reduced by running the reaction in solvents or in aqueous solvents. In the methods of the present invention, said nucleophile attacks the enzyme-substrate complex eventually forming a transacylated compound. On top of being a nuclophile, said reagent also has to comprise one or more functional groups, which are not accessible in the peptide to be conjugated.

CPY has specific requirements to the amino acid sequence of a peptide to be able to incorporate a nucleophile into the a C-terminal. The particular combination of CPY and GH-XX-Ala, and in particular GH-Leu-Ala is advantageously in that e.g. higher yield are obtained in the corresponding reaction between CPY and GH-Ala, while maintaining a close relationship to the GH. This aspect could be of importance if GH represents hGH. A close relationship to the natural peptide is generally regarded as an advantage with therapeutic interventions comprising administration of variants or analogues of this natural peptide as it minimizes the risk of e.g. any unwanted antibody generation.

Many nucleophilic compounds are known which could be incorporated into peptides according to the methods of the present invention, and α-amino acids is one such type of nucleophilic compounds. For the purpose of the present invention, it is, however, preferred to select the nucleophilic compound so that the transacylated compound formed is not itself a substrate for the enzyme applied. Stated differently, it is preferred to apply a nucleophilic compound which effectively blocks any further reaction of the enzyme. One example of such compounds is amides of α-amino acids as carboxy amidated peptides are not substrates for carboxypeptidases.

It is recognised that whether or not a compound is a substrate for a given enzyme in principle depends on the conditions, e.g. the time frame, under which the reaction takes place. Given sufficient time, many compounds are, in fact, substrates for an enzyme although they are not under normal conditions regarded as such. When it is stated above that the transacylated compound itself should not be a substrate of the enzyme it is intended to indicate that the tranacylated compound itself is not a substrate for the enzyme to an extent where the following reactions in the method of the present invention are disturbed. If the transacylated compound is, in fact, a substrate for the enzyme, the enzyme may be removed or inactivated, e.g. by enzyme inhibitors, following the transacylation reaction.

In one embodiment, the invention relates to a method of conjugating GH-XX-Ala, wherein GH-XX-Ala is reacted in one or more steps with a first compound, which is an α-amino acid amide represented by the formula

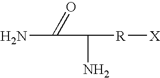

in the presence of CPY to form a transacylated compound of the formula

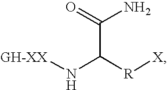

said transacylated peptide being further reacted in one or more steps with a second compound of the formula

Y-E-PEG

to form a conjugated GH of the formula

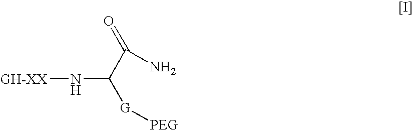

wherein G represent R-A-E, wherein R represents a linker or a bond; E represents a linker or a bond; A represents the moiety formed by the reaction between the functional groups comprised in X and Y; and
GH represent a growth hormone compound;
X represents a radical comprising a functional group not accessible in the amino acid residues constituting the GH;
Y represents a radical comprising one or more functional groups which groups react with functional groups present in X, and which functional groups do not react with functional groups accessible in the GH;
PEG represent a poly ethylene glycol moiety;
and XX represents an amino acid residue selected from histidine, asparatic acid, arginine, phenylalanine, alanine, glycine, glutamine, glutamic acid, lysine, leucine, methionine, asparagine, serine, tyrosine, threonine, isoleucine, tryptophane, proline, and valine.

In a further embodiment, the invention relates to methods of conjugating GH-XX-Ala as disclosed above, which further comprises the step of formulating the resulting conjugated peptide in a pharmaceutical composition.

Following the conjugation, the conjugated GH-XX-Ala may be isolated and purified by techniques well-known in the art. The conjugated peptide may also be converted into a pharmaceutically acceptable salt or prodrug, if relevant.

In one embodiment, XX represents Leu.

The moiety, A, formed in the reaction between the functional groups of X and Y may in principle be of any kind depending on what properties of the final conjugated peptide is desired. In some situation it may be desirable to have a labile bond which can be cleaved at some later stage, e.g. by some enzymatic action or by photolysis. In other situations, it may be desirable to have a stable bond, so that a stable conjugated peptide is obtained. Particular mentioning is made of the type of moieties formed by reactions between amine derivatives and carbonyl groups, such as oxime, hydrazone, phenylhydrazone and semicarbazone moieties.

In one embodiment the functional groups of X and Y are selected from amongst carbonyl groups, such as keto and aldehyde groups, and amino derivatives, such as

hydrazine derivatives —NH—NH₂,
hydrazine carboxylate derivatives —O—C(O)—NH—NH₂,
semicarbazide derivatives —NH—C(O)—NH—NH₂,
thiosemicarbazide derivatives —NH—C(S)—NH—NH₂,
carbonic acid dihydrazide derivatives —NHC(O)—NH—NH—C(O)—NH—NH₂,
carbazide derivatives —NH—NH—C(O)—NH—NH₂,
thiocarbazide derivatives —NH—NH—C(S)—NH—NH₂,
aryl hydrazine derivatives —NH—C(O)—C₆H₄—NH—NH₂, and
hydrazide derivatives —C(O)—NH—NH₂;
oxylamine derivatives, such as —O—NH₂, —C(O)—O—NH₂, —NH—C(O)—O—NH₂ and —NH—C(S)—O—NH₂.

It is to be understood, that if the functional group comprised in X is a carbonyl group, then the functional group comprised in Y is an amine derivative, and vice versa. Due to the presence of —NH₂ groups in most peptides, a better selectivity is believed to be obtained if X comprises a keto- or an aldehyde-functionality.

Another example of a suitable pair of X and Y is azide derivatives (—N₃) and alkynes (or vice versa) which react to form a triazole moiety.

Another example of a suitable pair of X and Y is alkyne and nitril-oxide (or vice versa), which reacts to form a isooxazolidine moiety.

Another example of a suitable pair of X and Y is alkyne and haloaryl (or vice versa), which reacts to form a aralkyne moiety.

Another example of a suitable pair of X and Y is alkyne and aryl trifluorosulphonate (or vice versa), which reacts to form a aralkyne moiety.

In particular, the group to be transacylated,

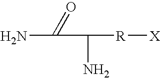

may be selected from amongst 2-amino-3-oxo-butyramide, 2-amino-6-(4-oxo-pentanoylamino)-hexanoic acid amide, 2-amino-3-(2-oxo-2-phenyl-ethylsulfanyl)-propionamide, 2-amino-5-oxo-hexanoic acid amide, 2-amino-3-oxo-propionamide, 2-amino-6-(4-acetylbenzoylamino)hexanoic acid amide, (2S)-2-amino-3-[4-(2-oxopropoxy)phenyl]propionamide, (2S)-2-amino-3-[4-(2-oxobutoxy)phenyl]propionamide, (2S)-2-amino-3-[4-(2-oxopentoxy)phenyl]propionamide, (2S)-2-amino-3-[4-(4-oxopentoxy)phenyl]propionamide, (2S)-2-amino-6-(4-oxo-4-phenylbutyrylamino)hexanoic acid amide, (2S)-2-amino-6-(4-oxo-4-(4-chlorophenylbutyrylamino)hexanoic acid amide, 3-acetyl-N-((5S)-5-amino-5-carbamoylpentyl)benzamide, 2-acetyl-N-((5S)-5-amino-5-carbamoylpentyl)benzamide, (2S)-2-amino-3-(4-(prop-2-ynyloxy)phenyl)propionamide, (S)-2-aminopent-4-ynoicacid amide, (2S)-2-amino-6-(30-(prop-2-ynyl)benzoylamino)hexanoic amide, (2S)-2-amino-6-(4-(prop-2-ynyl)benzoylamino)hexanoic amide, (2S)-2-amino-6-(2-(prop-2-ynyl)benzoylamino)hexanoic amide, (2S)-2-amino-3-[4-(1-oxoethyoxy)phenyl)propionamide and S-phenylacylcysteine amide.

Particular examples of Y-E-PEG includes

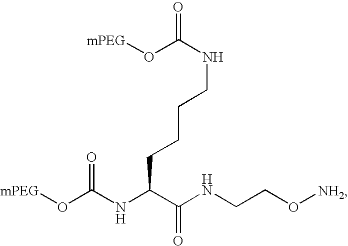

wherein mPEG has a molecular weight of around 10 kDa, around 20 kDa, around 30 kDa or around 40 kDa,

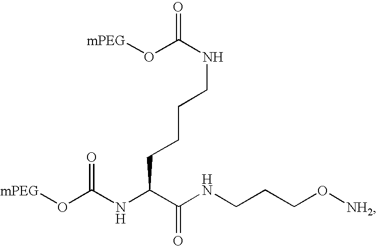

wherein mPEG has a molecular weight of around 10 kDa, around 20 kDa, around 30 kDa or around 40 kDa,

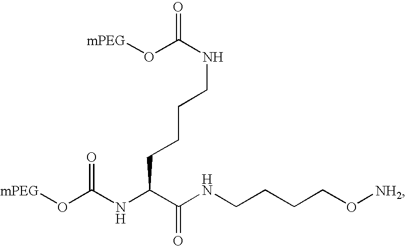

wherein mPEG has a molecular weight of around 10 kDa, around 20 kDa, around 30 kDa or around 40 kDa,

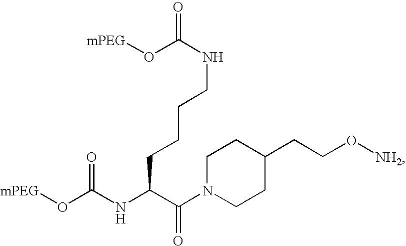

wherein mPEG has a molecular weight of around 10 kDa, around 20 kDa, around 30 kDa or around 40 kDa,

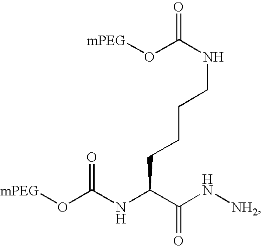

wherein mPEG has a molecular weight of around 10 kDa, around 20 kDa, around 30 kDa or around 40 kDa,

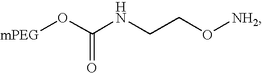

wherein mPEG has a molecular weight of around 10 kDa, around 20 kDa, around 30 kDa or around 40 kDa,

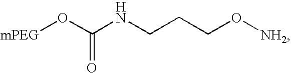

wherein mPEG has a molecular weight of around 10 kDa, around 20 kDa, around 30 kDa or around 40 kDa,

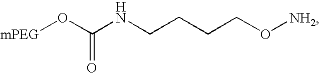

wherein mPEG has a molecular weight of around 10 kDa, around 20 kDa, around 30 kDa or around 40 kDa,

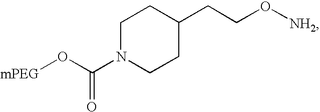

wherein mPEG has a molecular weight of around 10 kDa, around 20 kDa, around 30 kDa or around 40 kDa,

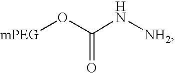

wherein mPEG has a molecular weight of around 10 kDa, around 20 kDa, around 30 kDa or around 40 kDa,

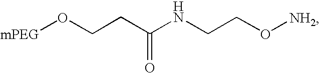

wherein mPEG has a molecular weight of around 10 kDa, around 20 kDa, around 30 kDa or around 40 kDa,

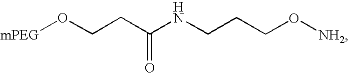

wherein mPEG has a molecular weight of around 10 kDa, around 20 kDa, around 30 kDa or around 40 kDa,

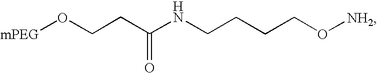

wherein mPEG has a molecular weight of around 10 kDa, around 20 kDa, around 30 kDa or around 40 kDa,

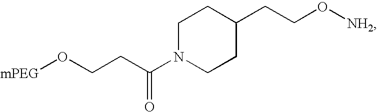

wherein mPEG has a molecular weight of around 10 kDa, around 20 kDa, around 30 kDa or around 40 kDa,

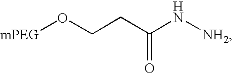

wherein mPEG has a molecular weight of around 10 kDa, around 20 kDa, around 30 kDa or around 40 kDa,

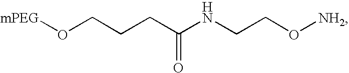

wherein mPEG has a molecular weight of around 10 kDa, around 20 kDa, around 30 kDa or around 40 kDa,

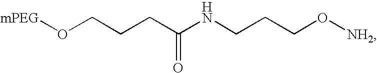

wherein mPEG has a molecular weight of around 10 kDa, around 20 kDa, around 30 kDa or around 40 kDa,

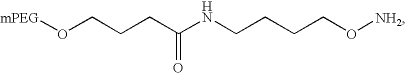

wherein mPEG has a molecular weight of around 10 kDa, around 20 kDa, around 30 kDa or around 40 kDa,

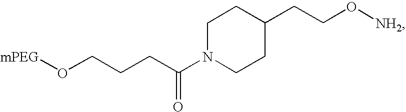

wherein mPEG has a molecular weight of around 10 kDa, around 20 kDa, around 30 kDa or around 40 kDa,

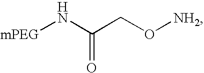

wherein mPEG has a molecular weight of around 10 kDa, around 20 kDa, around 30 kDa or around 40 kDa,

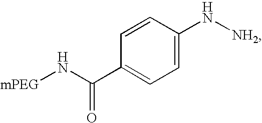

wherein mPEG has a molecular weight of around 10 kDa, around 20 kDa, around 30 kDa or around 40 kDa,

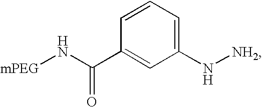

wherein mPEG has a molecular weight of around 10 kDa, around 20 kDa, around 30 kDa or around 40 kDa,

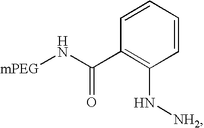

wherein mPEG has a molecular weight of around 10 kDa, around 20 kDa, around 30 kDa or around 40 kDa,

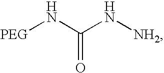

wherein mPEG has a molecular weight of around 10 kDa, around 20 kDa, around 30 kDa or around 40 kDa,

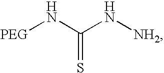

wherein mPEG has a molecular weight of around 10 kDa, around 20 kDa, around 30 kDa or around 40 kDa,

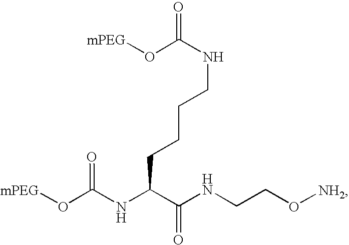

wherein mPEG has a molecular weight of around 10 kDa, around 20 kDa, around 30 kDa or around 40 kDa,

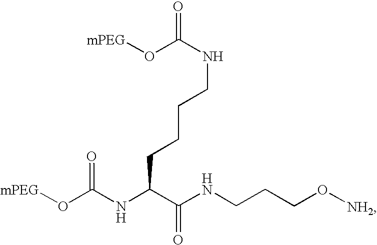

wherein mPEG has a molecular weight of around 10 kDa, around 20 kDa, around 30 kDa or around 40 kDa,

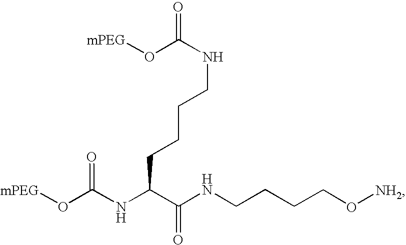

wherein mPEG has a molecular weight of around 10 kDa, around 20 kDa, around 30 kDa or around 40 kDa,

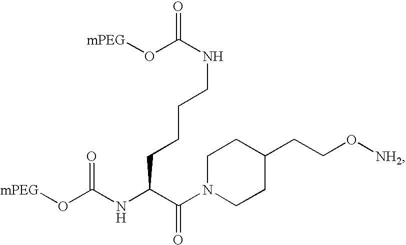

wherein mPEG has a molecular weight of around 10 kDa, around 20 kDa, around 30 kDa or around 40 kDa,

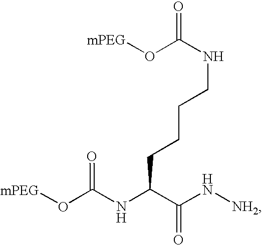

wherein mPEG has a molecular weight of around 10 kDa, around 20 kDa, around 30 kDa or around 40 kDa,

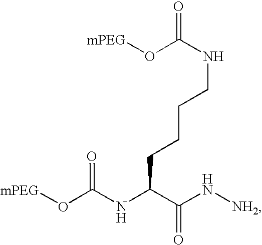

wherein mPEG has a molecular weight of around 10 kDa, around 20 kDa, around 30 kDa or around 40 kDa,

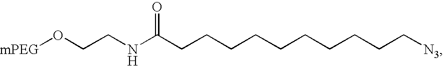

wherein mPEG has a molecular weight of around 10 kDa, around 20 kDa, around 30 kDa or around 40 kDa,

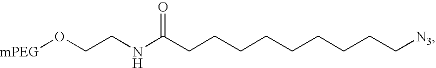

wherein mPEG has a molecular weight of around 10 kDa, around 20 kDa, around 30 kDa or around 40 kDa,

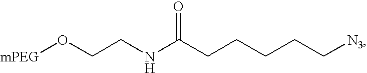

wherein mPEG has a molecular weight of around 10 kDa, around 20 kDa, around 30 kDa or around 40 kDa,

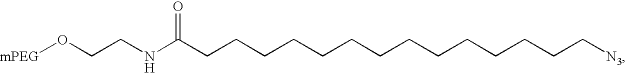

wherein mPEG has a molecular weight of around 10 kDa, around 20 kDa, around 30 kDa or around 40 kDa,

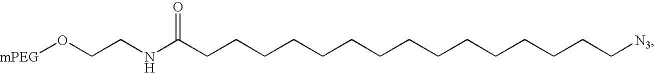

wherein mPEG has a molecular weight of around 10 kDa, around 20 kDa, around 30 kDa or around 40 kDa,

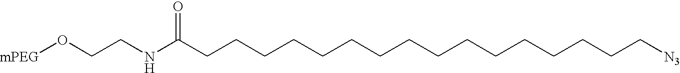

wherein mPEG has a molecular weight of around 10 kDa, around 20 kDa, around 30 kDa or around 40 kDa,

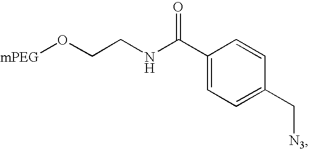

wherein mPEG has a molecular weight of around 10 kDa, around 20 kDa, around 30 kDa or around 40 kDa,

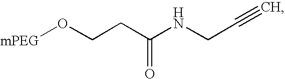

wherein mPEG has a molecular weight of around 10 kDa, around 20 kDa, around 30 kDa or around 40 kDa,

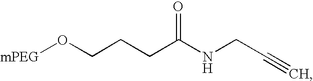

wherein mPEG has a molecular weight of around 10 kDa, around 20 kDa, around 30 kDa or around 40 kDa,

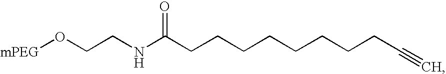

wherein mPEG has a molecular weight of around 10 kDa, around 20 kDa, around 30 kDa or around 40 kDa,

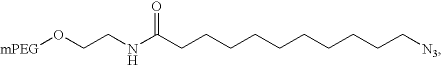

wherein mPEG has a molecular weight of around 10 kDa, around 20 kDa, around 30 kDa or around 40 kDa,

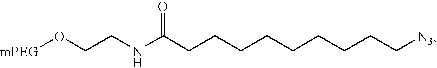

wherein mPEG has a molecular weight of around 10 kDa, around 20 kDa, around 30 kDa or around 40 kDa,

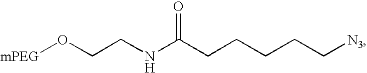

wherein mPEG has a molecular weight of around 10 kDa, around 20 kDa, around 30 kDa or around 40 kDa,

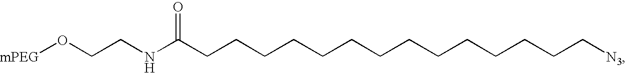

wherein mPEG has a molecular weight of around 10 kDa, around 20 kDa, around 30 kDa or around 40 kDa,

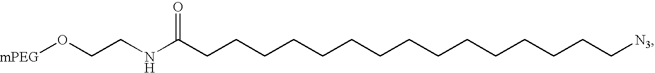

wherein mPEG has a molecular weight of around 10 kDa, around 20 kDa, around 30 kDa or around 40 kDa,

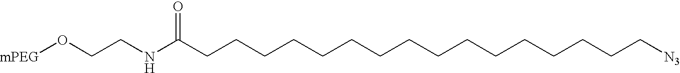

wherein mPEG has a molecular weight of around 10 kDa, around 20 kDa, around 30 kDa or around 40 kDa,

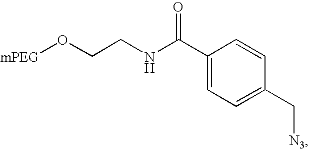

wherein mPEG has a molecular weight of around 10 kDa, around 20 kDa, around 30 kDa or around 40 kDa,

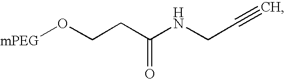

wherein mPEG has a molecular weight of around 10 kDa, around 20 kDa, around 30 kDa or around 40 kDa,

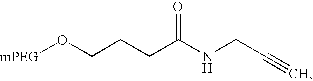

wherein mPEG has a molecular weight of around 10 kDa, around 20 kDa, around 30 kDa or around 40 kDa,

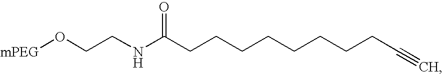

wherein mPEG has a molecular weight of around 10 kDa, around 20 kDa, around 30 kDa or around 40 kDa,

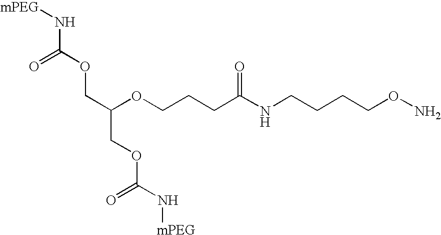

wherein mPEG has a molecular weight of around 10 kDa, around 20 kDa, around 30 kDa or around 40 kDa,

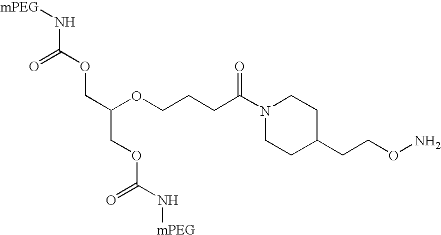

wherein mPEG has a molecular weight of around 10 kDa, around 20 kDa, around 30 kDa or around 40 kDa,

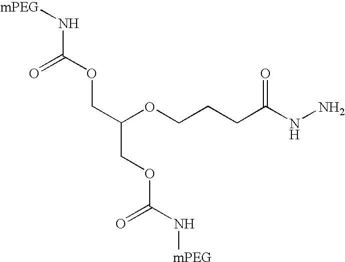

wherein mPEG has a molecular weight of around 10 kDa, around 20 kDa, around 30 kDa or around 40 kDa,

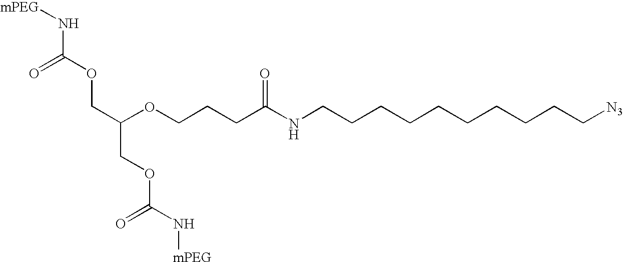

wherein mPEG has a molecular weight of around 10 kDa, around 20 kDa, around 30 kDa or around 40 kDa,

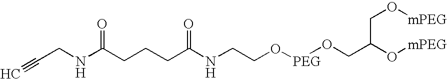

wherein mPEG has a molecular weight of around 10 kDa, around 20 kDa, around 30 kDa or around 40 kDa,

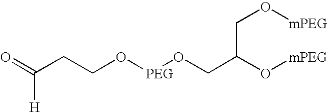

wherein mPEG has a molecular weight of around 10 kDa, around 20 kDa, around 30 kDa or around 40 kDa, and

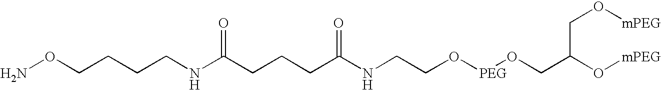

wherein mPEG has a molecular weight of around 10 kDa, around 20 kDa, around 30 kDa or around 40 kDa.

In one embodiment, the invention relates to growth hormone compounds suitable for the conjugation according to the methods of the present invention, i.e. to compounds of the formula GH-XX-Ala, wherein XX represents an amino acid residue selected from histidine, asparatic acid, arginine, phenylalanine, alanine, glycine, glutamine, glutamic acid, lysine, leucine, methionine, asparagine, serine, tyrosine, threonine, isoleucine, tryptophane, proline, and valine. Particular mentioning is made of hGH-Leu-Ala.

### Pharmaceutical Compositions

Another purpose is to provide a pharmaceutical composition comprising a conjugated GH of the present invention which is present in a concentration from 10⁻¹⁵ mg/ml to 200 mg/ml, such as e.g. 10⁻¹⁰ mg/ml to 5 mg/ml and wherein said composition has a pH from 2.0 to 10.0. The composition may further comprise pharmaceutical exhibients, such as a buffer system, preservative(s), tonicity agent(s), chelating agent(s), stabilizers and surfactants. In one embodiment of the invention the pharmaceutical composition is an aqueous composition, i.e. composition comprising water. Such composition is typically a solution or a suspension. In a further embodiment of the invention the pharmaceutical composition is an aqueous solution. The term “aqueous composition” is defined as a composition comprising at least 50% w/w water. Likewise, the term “aqueous solution” is defined as a solution comprising at least 50% w/w water, and the term “aqueous suspension” is defined as a suspension comprising at least 50% w/w water.

In another embodiment the pharmaceutical composition is a freeze-dried composition, whereto the physician or the patient adds solvents and/or diluents prior to use.

In another embodiment the pharmaceutical composition is a dried composition (e.g. freeze-dried or spray-dried) ready for use without any prior dissolution.

In a further aspect the invention relates to a pharmaceutical composition comprising an aqueous solution of a GH conjugate, and a buffer, wherein said GH conjugate is present in a concentration from 0.1-100 mg/ml or above, and wherein said composition has a pH from about 2.0 to about 10.0.

In a another embodiment of the invention the pH of the composition is selected from the list consisting of 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, and 10.0.

In a further embodiment of the invention the buffer is selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid or mixtures thereof. Each one of these specific buffers constitutes an alternative embodiment of the invention.

In a further embodiment of the invention the composition further comprises a pharmaceutically acceptable preservative. In a further embodiment of the invention the preservative is selected from the group consisting of phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, bronopol, benzoic acid, imidurea, chlorohexidine, sodium dehydroacetate, chlorocresol, ethyl p-hydroxybenzoate, benzethonium chloride, chlorphenesine (3p-chlorphenoxypropane-1,2-diol) or mixtures thereof. In a further embodiment of the invention the preservative is present in a concentration from 0.1 mg/ml to 20 mg/ml. In a further embodiment of the invention the preservative is present in a concentration from 0.1 mg/ml to 5 mg/ml. In a further embodiment of the invention the preservative is present in a concentration from 5 mg/ml to 10 mg/ml. In a further embodiment of the invention the preservative is present in a concentration from 10 mg/ml to 20 mg/ml. Each one of these specific preservatives constitutes an alternative embodiment of the invention. The use of a preservative in pharmaceutical compositions is well-known to the skilled person. For convenience reference is made to Remington: The Science and Practice of Pharmacy, 20^{th} edition, 2000.

In a further embodiment of the invention the composition further comprises an isotonic agent. In a further embodiment of the invention the isotonic agent is selected from the group consisting of a salt (e.g. sodium chloride), a sugar or sugar alcohol, an amino acid (e.g. L-glycine, L-histidine, arginine, lysine, isoleucine, aspartic acid, tryptophan, threonine), an alditol (e.g. glycerol (glycerine), 1,2-propanediol (propyleneglycol), 1,3-propanediol, 1,3-butanediol) polyethyleneglycol (e.g. PEG400), or mixtures thereof. Any sugar such as mono-, di-, or polysaccharides, or water-soluble glucans, including for example fructose, glucose, mannose, sorbose, xylose, maltose, lactose, sucrose, trehalose, dextran, pullulan, dextrin, cyclodextrin, soluble starch, hydroxyethyl starch and carboxymethylcellulose-Na may be used. In one embodiment the sugar additive is sucrose. Sugar alcohol is defined as a C₄-C₈ hydrocarbon having at least one —OH group and includes, for example, mannitol, sorbitol, inositol, galactitol, dulcitol, xylitol, and arabitol. In one embodiment the sugar alcohol additive is mannitol. The sugars or sugar alcohols mentioned above may be used individually or in combination. There is no fixed limit to the amount used, as long as the sugar or sugar alcohol is soluble in the liquid preparation and does not adversely effect the stabilizing effects obtained using the methods of the invention. In one embodiment, the sugar or sugar alcohol concentration is between about 1 mg/ml and about 150 mg/ml. In a further embodiment of the invention the isotonic agent is present in a concentration from 1 mg/ml to 50 mg/ml. In a further embodiment of the invention the isotonic agent is present in a concentration from 1 mg/ml to 7 mg/ml. In a further embodiment of the invention the isotonic agent is present in a concentration from 8 mg/ml to 24 mg/ml. In a further embodiment of the invention the isotonic agent is present in a concentration from 25 mg/ml to 50 mg/ml. Each one of these specific isotonic agents constitutes an alternative embodiment of the invention. The use of an isotonic agent in pharmaceutical compositions is well-known to the skilled person. For convenience reference is made to Remington: The Science and Practice of Pharmacy, 20^{th} edition, 2000.

In a further embodiment of the invention the composition further comprises a chelating agent. In a further embodiment of the invention the chelating agent is selected from salts of ethylenediaminetetraacetic acid (EDTA), citric acid, and aspartic acid, and mixtures thereof. In a further embodiment of the invention the chelating agent is present in a concentration from 0.1 mg/ml to 5 mg/ml. In a further embodiment of the invention the chelating agent is present in a concentration from 0.1 mg/ml to 2 mg/ml. In a further embodiment of the invention the chelating agent is present in a concentration from 2 mg/ml to 5 mg/ml. Each one of these specific chelating agents constitutes an alternative embodiment of the invention. The use of a chelating agent in pharmaceutical compositions is well-known to the skilled person. For convenience reference is made to Remington: The Science and Practice of Pharmacy, 20^{th} edition, 2000.

In a further embodiment of the invention the composition further comprises a stabilizer. The use of a stabilizer in pharmaceutical compositions is well-known to the skilled per-son. For convenience reference is made to Remington: The Science and Practice of Pharmacy, 20^{th} edition, 2000.

More particularly, compositions of the invention are stabilized liquid pharmaceutical compositions whose therapeutically active components include a protein that possibly exhibits aggregate formation during storage in liquid pharmaceutical compositions. By “aggregate formation” is intended a physical interaction between the protein molecules that results in formation of oligomers, which may remain soluble, or large visible aggregates that precipitate from the solution. By “during storage” is intended a liquid pharmaceutical composition or composition once prepared, is not immediately administered to a subject. Rather, following preparation, it is packaged for storage, either in a liquid form, in a frozen state, or in a dried form for later reconstitution into a liquid form or other form suitable for administration to a subject. By “dried form” is intended the liquid pharmaceutical composition or composition is dried either by freeze drying (i.e., lyophilization; see, for example, Williams and Polli (1984) J. Parenteral Sci. Technol. 38:48-59), spray drying (see Masters (1991) in Spray-Drying Handbook (5th ed; Longman Scientific and Technical, Essez, U.K.), pp. 491-676; Broadhead et al. (1992) Drug Devel. Ind. Pharm. 18:1169-1206; and Mumenthaler et al. (1994) Pharm. Res. 11:12-20), or air drying (Carpenter and Crowe (1988) Cryobiology 25:459-470; and Roser (1991) Biopharm. 4:47-53). Aggregate formation by a protein during storage of a liquid pharmaceutical composition can adversely affect biological activity of that protein, resulting in loss of therapeutic efficacy of the pharmaceutical composition. Furthermore, aggregate formation may cause other problems such as blockage of tubing, membranes, or pumps when the protein-containing pharmaceutical composition is administered using an infusion system.

The pharmaceutical compositions of the invention may further comprise an amount of an amino acid base sufficient to decrease aggregate formation by the protein during storage of the composition. By “amino acid base” is intended an amino acid or a combination of amino acids, where any given amino acid is present either in its free base form or in its salt form. Where a combination of amino acids is used, all of the amino acids may be present in their free base forms, all may be present in their salt forms, or some may be present in their free base forms while others are present in their salt forms. In one embodiment, amino acids to use in preparing the compositions of the invention are those carrying a charged side chain, such as arginine, lysine, aspartic acid, and glutamic acid. Any stereoisomer (i.e., L or D isomer, or mixtures thereof) of a particular amino acid (methionine, histidine, arginine, lysine, isoleucine, aspartic acid, tryptophan, threonine and mixtures thereof) or combinations of these stereoisomers or glycine or an organic base such as but not limited to imidazole, may be present in the pharmaceutical compositions of the invention so long as the particular amino acid or organic base is present either in its free base form or its salt form. In one embodiment the L-stereoisomer of an amino acid is used. In one embodiment the L-stereoisomer is used. Compositions of the invention may also be formulated with analogues of these amino acids. By “amino acid analogue” is intended a derivative of the naturally occurring amino acid that brings about the desired effect of decreasing aggregate formation by the protein during storage of the liquid pharmaceutical compositions of the invention. Suitable arginine analogues include, for example, aminoguanidine, ornithine and N-monoethyl L-arginine, suitable methionine analogues include ethionine and buthionine and suitable cysteine analogues include S-methyl-L cysteine. As with the other amino acids, the amino acid analogues are incorporated into the compositions in either their free base form or their salt form. In a further embodiment of the invention the amino acids or amino acid analogues are used in a concentration, which is sufficient to prevent or delay aggregation of the protein.

In a further embodiment of the invention methionine (or other sulphuric amino acids or amino acid analogous) may be added to inhibit oxidation of methionine residues to methionine sulfoxide when the protein acting as the therapeutic agent is a protein comprising at least one methionine residue susceptible to such oxidation. By “inhibit” is intended minimal accumulation of methionine oxidized species over time. Inhibiting methionine oxidation results in greater retention of the protein in its proper molecular form. Any stereoisomer of methionine (L or D isomer) or any combinations thereof can be used. The amount to be added should be an amount sufficient to inhibit oxidation of the methionine residues such that the amount of methionine sulfoxide is acceptable to regulatory agencies. Typically, this means that the composition contains no more than about 10% to about 30% methionine sulfoxide. Generally, this can be obtained by adding methionine such that the ratio of methionine added to methionine residues ranges from about 1:1 to about 1000:1, such as 10:1 to about 100:1.

In a further embodiment of the invention the composition further comprises a stabilizer selected from the group of high molecular weight polymers or low molecular compounds. In a further embodiment of the invention the stabilizer is selected from polyethylene glycol (e.g. PEG 3350), polyvinyl alcohol (PVA), polyvinylpyrrolidone, carboxy/hydroxycellulose or derivates thereof (e.g. HPC, HPC-SL, HPC-L and HPMC), cyclodextrins, sulphur-containing substances as monothioglycerol, thioglycolic acid and 2-methylthioethanol, and different salts (e.g. sodium chloride). Each one of these specific stabilizers constitutes an alternative embodiment of the invention.

The pharmaceutical compositions may also comprise additional stabilizing agents, which further enhance stability of a therapeutically active protein therein. Stabilizing agents of particular interest to the present invention include, but are not limited to, methionine and EDTA, which protect the protein against methionine oxidation, and a nonionic surfactant, which protects the protein against aggregation associated with freeze-thawing or mechanical shearing.

In a further embodiment of the invention the composition further comprises a surfactant. In a further embodiment of the invention the surfactant is selected from a detergent, ethoxylated castor oil, polyglycolyzed glycerides, acetylated monoglycerides, sorbitan fatty acid esters, polyoxypropylene-polyoxyethylene block polymers (eg. poloxamers such as Pluronic® F68, poloxamer 188 and 407, Triton X-100), polyoxyethylene sorbitan fatty acid esters, polyoxyethylene and polyethylene derivatives such as alkylated and alkoxylated derivatives (tweens, e.g. Tween-20, Tween-40, Tween-80 and Brij-35), monoglycerides or ethoxylated derivatives thereof, diglycerides or polyoxyethylene derivatives thereof, alcohols, glycerol, lectins and phospholipids (eg. phosphatidyl serine, phosphatidyl choline, phosphatidyl ethanolamine, phosphatidyl inositol, diphosphatidyl glycerol and sphingomyelin), derivates of phospholipids (eg. dipalmitoyl phosphatidic acid) and lysophospholipids (eg. palmitoyl lysophosphatidyl-L-serine and 1-acyl-sn-glycero-3-phosphate esters of ethanolamine, choline, serine or threonine) and alkyl, alkoxyl (alkyl ester), alkoxy (alkyl ether)-derivatives of lysophosphatidyl and phosphatidylcholines, e.g. lauroyl and myristoyl derivatives of lysophosphatidylcholine, dipalmitoylphosphatidylcholine, and modifications of the polar head group, that is cholines, ethanolamines, phosphatidic acid, serines, threonines, glycerol, inositol, and the positively charged DODAC, DOTMA, DCP, BISHOP, lysophosphatidylserine and lysophosphatidylthreonine, and glycerophospholipids (eg. cephalins), glyceroglycolipids (eg. galactopyransoide), sphingoglycolipids (eg. ceramides, gangliosides), dodecylphosphocholine, hen egg lysolecithin, fusidic acid derivatives—(e.g. sodium tauro-dihydrofusidate etc.), long-chain fatty acids and salts thereof C₆-C₁₂ (eg. oleic acid and caprylic acid), acylcarnitines and derivatives, N^{α}-acylated derivatives of lysine, arginine or histidine, or side-chain acylated derivatives of lysine or arginine, N′-acylated derivatives of dipeptides comprising any combination of lysine, arginine or histidine and a neutral or acidic amino acid, N′-acylated derivative of a tripeptide comprising any combination of a neutral amino acid and two charged amino acids, DSS (docusate sodium, CAS registry no [577-11-7]), docusate calcium, CAS registry no [128-49-4]), docusate potassium, CAS registry no [7491-09-0]), SDS (sodium dodecyl sulphate or sodium lauryl sulphate), sodium caprylate, cholic acid or derivatives thereof, bile acids and salts thereof and glycine or taurine conjugates, ursodeoxycholic acid, sodium cholate, sodium deoxycholate, sodium taurocholate, sodium glycocholate, N-Hexadecyl-N,N-dimethyl-3-ammonio-1-propanesulfonate, anionic (alkyl-aryl-sulphonates) monovalent surfactants, zwifterionic surfactants (e.g. N-alkyl-N,N-dimethylammonio-1-propanesulfonates, 3-cholamido-1-propyldimethylammonio-1-propanesulfonate, cationic surfactants (quaternary ammonium bases) (e.g. cetyl-trimethylammonium bromide, cetylpyridinium chloride), non-ionic surfactants (eg. Dodecyl β-D-glucopyranoside), poloxamines (eg. Tetronic's), which are tetrafunctional block copolymers derived from sequential addition of propylene oxide and ethylene oxide to ethylenediamine, or the surfactant may be selected from the group of imidazoline derivatives, or mixtures thereof. Each one of these specific surfactants constitutes an alternative embodiment of the invention.

The use of a surfactant in pharmaceutical compositions is well-known to the skilled person. For convenience reference is made to Remington: The Science and Practice of Pharmacy, 20^{th} edition, 2000.

It is possible that other ingredients may be present in the pharmaceutical composition of the present invention. Such additional ingredients may include wetting agents, emulsifiers, antioxidants, bulking agents, tonicity modifiers, chelating agents, metal ions, oleaginous vehicles, proteins (e.g., human serum albumin, gelatine or proteins) and a zwitterion (e.g., an amino acid such as betaine, taurine, arginine, glycine, lysine and histidine). Such additional ingredients, of course, should not adversely affect the overall stability of the pharmaceutical composition of the present invention.

Pharmaceutical compositions containing a GH conjugate according to the present invention may be administered to a patient in need of such treatment at several sites, for example, at topical sites, for example, skin and mucosal sites, at sites which bypass absorption, for example, administration in an artery, in a vein, in the heart, and at sites which involve absorption, for example, administration in the skin, under the skin, in a muscle or in the abdomen.

Administration of pharmaceutical compositions according to the invention may be through several routes of administration, for example, lingual, sublingual, buccal, in the mouth, oral, in the stomach and intestine, nasal, pulmonary, for example, through the bronchioles and alveoli or a combination thereof, epidermal, dermal, transdermal, vaginal, rectal, ocular, for examples through the conjunctiva, uretal, and parenteral to patients in need of such a treatment.

Compositions of the current invention may be administered in several dosage forms, for example, as solutions, suspensions, emulsions, microemulsions, multiple emulsion, foams, salves, pastes, plasters, ointments, tablets, coated tablets, rinses, capsules, for example, hard gelatine capsules and soft gelatine capsules, suppositories, rectal capsules, drops, gels, sprays, powder, aerosols, inhalants, eye drops, ophthalmic ointments, ophthalmic rinses, vaginal pessaries, vaginal rings, vaginal ointments, injection solution, in situ transforming solutions, for example in situ gelling, in situ setting, in situ precipitating, in situ crystallization, infusion solution, and implants.

Compositions of the invention may further be compounded in, or attached to, for example through covalent, hydrophobic and electrostatic interactions, a drug carrier, drug delivery system and advanced drug delivery system in order to further enhance stability of the GH conjugate, increase bioavailability, increase solubility, decrease adverse effects, achieve chronotherapy well known to those skilled in the art, and increase patient compliance or any combination thereof. Examples of carriers, drug delivery systems and advanced drug delivery systems include, but are not limited to, polymers, for example cellulose and derivatives, polysaccharides, for example dextran and derivatives, starch and derivatives, poly(vinyl alcohol), acrylate and methacrylate polymers, polylactic and polyglycolic acid and block copolymers thereof, polyethylene glycols, carrier proteins, for example albumin, gels, for example, thermogelling systems, for example block co-polymeric systems well known to those skilled in the art, micelles, liposomes, microspheres, nanoparticulates, liquid crystals and dispersions thereof, L2 phase and dispersions there of, well known to those skilled in the art of phase behaviour in lipid-water systems, polymeric micelles, multiple emulsions, self-emulsifying, self-microemulsifying, cyclodextrins and derivatives thereof, and dendrimers.

Compositions of the current invention are useful in the composition of solids, semi-solids, powder and solutions for pulmonary administration of GH conjugate, using, for example a metered dose inhaler, dry powder inhaler and a nebulizer, all being devices well known to those skilled in the art.

Compositions of the current invention are specifically useful in the composition of controlled, sustained, protracting, retarded, and slow release drug delivery systems. More specifically, but not limited to, compositions are useful in composition of parenteral controlled release and sustained release systems (both systems leading to a many-fold reduction in number of administrations), well known to those skilled in the art. Even more preferably, are controlled release and sustained release systems administered subcutaneous. Without limiting the scope of the invention, examples of useful controlled release system and compositions are hydrogels, oleaginous gels, liquid crystals, polymeric micelles, microspheres, nanoparticles,

Methods to produce controlled release systems useful for compositions of the current invention include, but are not limited to, crystallization, condensation, co-crystallization, precipitation, co-precipitation, emulsification, dispersion, high pressure homogenisation, encapsulation, spray drying, microencapsulating, coacervation, phase separation, solvent evaporation to produce microspheres, extrusion and supercritical fluid processes. General reference is made to Handbook of Pharmaceutical Controlled Release (Wise, D. L., ed. Marcel Dekker, New York, 2000) and Drug and the Pharmaceutical Sciences vol. 99: Protein Composition and Delivery (MacNally, E. J., ed. Marcel Dekker, New York, 2000).

Parenteral administration may be performed by subcutaneous, intramuscular, intraperitoneal or intravenous injection by means of a syringe, optionally a pen-like syringe. Alternatively, parenteral administration can be performed by means of an infusion pump. A further option is a composition which may be a solution or suspension for the administration of the GH conjugate in the form of a nasal or pulmonal spray. As a still further option, the pharmaceutical compositions containing the GH conjugate of the invention can also be adapted to transdermal administration, e.g. by needle-free injection or from a patch, optionally an iontophoretic patch, or transmucosal, e.g. buccal, administration.

The term “stabilized composition” refers to a composition with increased physical stability, increased chemical stability or increased physical and chemical stability.

The term “physical stability” of the protein composition as used herein refers to the tendency of the protein to form biologically inactive and/or insoluble aggregates of the protein as a result of exposure of the protein to thermo-mechanical stresses and/or interaction with interfaces and surfaces that are destabilizing, such as hydrophobic surfaces and interfaces. Physical stability of the aqueous protein compositions is evaluated by means of visual inspection and/or turbidity measurements after exposing the composition filled in suitable containers (e.g. cartridges or vials) to mechanical/physical stress (e.g. agitation) at different temperatures for various time periods. Visual inspection of the compositions is performed in a sharp focused light with a dark background. The turbidity of the composition is characterized by a visual score ranking the degree of turbidity for instance on a scale from 0 to 3 (a composition showing no turbidity corresponds to a visual score 0, and a composition showing visual turbidity in daylight corresponds to visual score 3). A composition is classified physical unstable with respect to protein aggregation, when it shows visual turbidity in daylight. Alternatively, the turbidity of the composition can be evaluated by simple turbidity measurements well-known to the skilled person. Physical stability of the aqueous protein compositions can also be evaluated by using a spectroscopic agent or probe of the conformational status of the protein. The probe is preferably a small molecule that preferentially binds to a non-native conformer of the protein. One example of a small molecular spectroscopic probe of protein structure is Thioflavin T. Thioflavin T is a fluorescent dye that has been widely used for the detection of amyloid fibrils. In the presence of fibrils, and perhaps other protein configurations as well, Thioflavin T gives rise to a new excitation maximum at about 450 nm and enhanced emission at about 482 nm when bound to a fibril protein form. Unbound Thioflavin T is essentially non-fluorescent at the wavelengths.

Other small molecules can be used as probes of the changes in protein structure from native to non-native states. For instance the “hydrophobic patch” probes that bind preferentially to exposed hydrophobic patches of a protein. The hydrophobic patches are generally buried within the tertiary structure of a protein in its native state, but become exposed as a protein begins to unfold or denature. Examples of these small molecular, spectroscopic probes are aromatic, hydrophobic dyes, such as antrhacene, acridine, phenanthroline or the like. Other spectroscopic probes are metal-amino acid complexes, such as cobalt metal complexes of hydrophobic amino acids, such as phenylalanine, leucine, isoleucine, methionine, and valine, or the like.

The term “chemical stability” of the protein composition as used herein refers to chemical covalent changes in the protein structure leading to formation of chemical degradation products with potential less biological potency and/or potential increased immunogenic properties compared to the native protein structure. Various chemical degradation products can be formed depending on the type and nature of the native protein and the environment to which the protein is exposed. Elimination of chemical degradation can most probably not be completely avoided and increasing amounts of chemical degradation products is often seen during storage and use of the protein composition as well-known by the person skilled in the art. Most proteins are prone to deamidation, a process in which the side chain amide group in glutaminyl or asparaginyl residues is hydrolysed to form a free carboxylic acid. Other degradations pathways involves formation of high molecular weight transformation products where two or more protein molecules are covalently bound to each other through transamidation and/or disulfide interactions leading to formation of covalently bound dimer, oligomer and polymer degradation products (Stability of Protein Pharmaceuticals, Ahern. T. J. & Manning M. C., Plenum Press, New York 1992). Oxidation (of for instance methionine residues) can be mentioned as another variant of chemical degradation. The chemical stability of the protein composition can be evaluated by measuring the amount of the chemical degradation products at various time-points after exposure to different environmental conditions (the formation of degradation products can often be accelerated by for instance increasing temperature). The amount of each individual degradation product is often determined by separation of the degradation products depending on molecule size and/or charge using various chromatography techniques (e.g. SEC-HPLC and/or RP-HPLC).

Hence, as outlined above, a “stabilized composition” refers to a composition with increased physical stability, increased chemical stability or increased physical and chemical stability. In general, a composition must be stable during use and storage (in compliance with recommended use and storage conditions) until the expiration date is reached.

In one embodiment of the invention the pharmaceutical composition comprising the GH conjugate is stable for more than 6 weeks of usage and for more than 3 years of storage.

In another embodiment of the invention the pharmaceutical composition comprising the GH conjugate is stable for more than 4 weeks of usage and for more than 3 years of storage.

In a further embodiment of the invention the pharmaceutical composition comprising the GH conjugateis stable for more than 4 weeks of usage and for more than two years of storage.

In an even further embodiment of the invention the pharmaceutical composition comprising the GH conjugate is stable for more than 2 weeks of usage and for more than two years of storage.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law).

All headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law.

### EXAMPLES

### HPLC-Methods

### Method 02-B1-2:

The RP-analyses was performed using an Alliance Waters 2695 system fitted with a Waters 2487 dualband detector. UV detections at 214 nm and 254 nm were collected using a Symmetry300 C18, 5 um, 3.9 mm×150 mm column, 42° C. The compounds are eluted with a linear gradient of 0-60% acetonitrile in water which is buffered with 0.05% trifluoroacetic acid over 15 minutes at a flow-rate of 1.0 min/min.

### Method 02-B4-4:

The RP-analyses was performed using an Alliance Waters 2695 system fitted with a Waters 2487 dualband detector. UV detections at 214 nm and 254 nm were collected using a Symmetry300 C18, 5 um, 3.9 mm×150 mm column, 42° C. The compounds are eluted with a linear gradient of 5-95% acetonitrile in water which is buffered with 0.05% trifluoroacetic acid over 15 minutes at a flow-rate of 1.0 min/min.

### Method 03-B1-1:

The RP-analysis was performed using a Waters 2690 systems fitted with a Waters 996 diode array detector. UV detections were collected at 214, 254, 276, and 301 nm on a 218TP54 4.6 mm×250 mm 5μ C-18 silica column (The Seperations Group, Hesperia), which was eluted at 1 ml/min at 42° C. The column was equilibrated with 5% acetonitrile, which was buffered with 0.1% trifluoroacetic acid, in a 0.1% aqueous solution of trifluoroacetic acid in water. After injection, the sample was eluted by a gradient of 0% to 90% acetonitrile, which was buffered with 0.1% trifluoroacetic acid, in a 0.1% aqueous solution of trifluoroacetic acid in water during 50 min.

Mass spectra for peptides were obtained on an Agilent 1100 Series in the range of 500-1800 Da or on Perkin Elmer PE API 100 in the range of 500-2000 Da. Typically the found signals for m/z correspond to a series of any of z=1, 2, 3, 4, 5, or 6.

MALDI-TOF spectra were obtained on a Bruker Daltonix autoflex.
Following abbreviations are used:

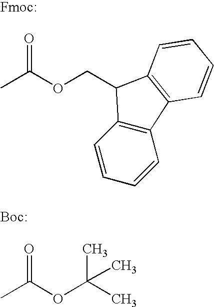

### DMSO: Dimethylsulfoxide

CHCA: 4-Hydroxy-alpha-cyanocinnamic acid

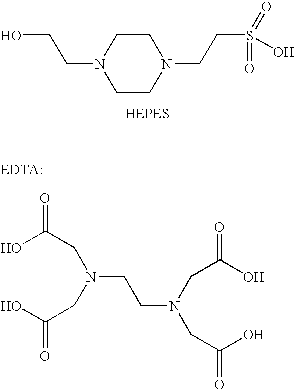

### CPY: Carboxypeptidase Y.

PMSF: Phenylmethanesulfonyl fluoride.

The transacylating compound, e.g. the compound of the formula

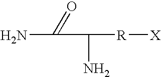

and the conjugating moiety, Y-E-PEG may either be acquired commercially or synthesized according to the following guidelines in general methods below.

### General Method (A):

A compound of the general formula

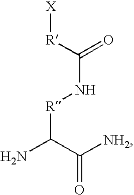

wherein R′ and R″ independently represents C₁₋₁₅alkylene, C₂₋₁₅alkenylene, C₂₋₁₅alkynylene, C₁₋₁₅heteroalkylene, C₂₋₁₅heteroalkenylene, C₂₋₁₅heteroalkynylene, wherein one or more homocyclic aromatic compound biradical or heterocyclic compound biradical may be inserted, may be prepared from a suitable amino acid methyl ester which is protected at the alpha-amino group by a suitable protecting group PG as described in the literature (e.g. T. W. Greene, P. G. M. Wuts, Protective groups in organic synthesis, 2^{nd} ed., 1991 John Wiley & Sons, Inc. New York)

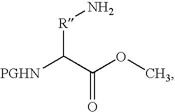

by an acylation method, e.g. using an suitable acid, in which X may or may not be protected by a suitable protective group, as described in the literature (e.g. T. W. Greene, P. G. M. Wuts, Protective groups in organic synthesis, 2^{nd} ed., 1991 John Wiley & Sons, Inc. New York)

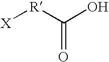

and a coupling reagent such as e.g. 1-hydroxybenzotriazole, 3,4-dihydro-3-hydroxybenzotriazin-4-one or 7-azabenzotriazole in combination with e.g. a carbodiimide such as e.g. diisopropylcarbodiimide or 1-(3-dimethylaminopropyl)-3-ethylcarbodiimide hydrochloride in the presence or absence of a suitable base such as e.g. triethylamine or ethyldiisopropylamine to form the ester of type

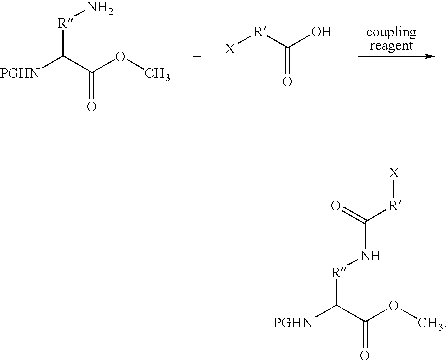

The ester may be transformed into the corresponding amide by reaction with e.g. ammonia in a suitable solvent or mixture of solvents such as e.g. water or N,N-dimethylformamide.

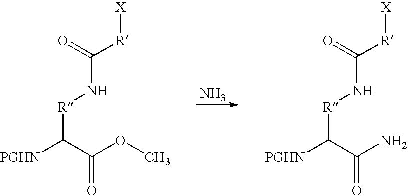

The removal of all protective groups may be performed in one or several steps by methods as described in the literature (e.g. T. W. Greene, P. G. M. Wuts, Protective groups in organic synthesis, 2^{nd} ed., 1991 John Wiley & Sons, Inc. New York)

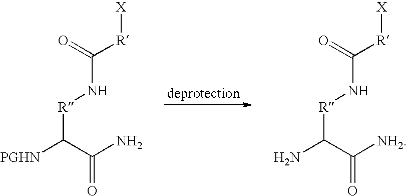

As defined in General Method (A)

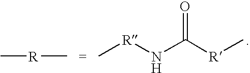

Amino acid methyl esters are generally commercially available, or they may be synthesized by well-known methods.

### General Method (B):

A compound of the general formula

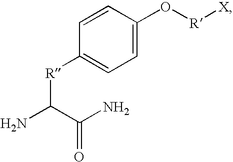

wherein R′ and R″ are defined as above, may be prepared from a suitable amino acid methyl ester which is protected at the alpha-amino group by a suitable protecting group PG, as described in the literature (e.g. T. W. Greene, P. G. M. Wuts, Protective groups in organic synthesis, 2^{nd} ed., 1991 John Wiley & Sons, Inc. New York)

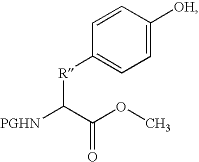

by an alkylation of the aromatic hydroxyl group using an suitable alcohol, in which X may or may not be protected by a suitable protective group, as described in the literature (e.g. T. W. Greene, P. G. M. Wuts, Protective groups in organic synthesis, 2^{nd} ed., 1991 John Wiley & Sons, Inc. New York)

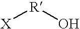

under conditions which effect alkylation, as described in the literature, e.g. Mitsunobu conditions such as e.g. triphenylphosphine and ethyl azodicarboxylate to form the ester of type

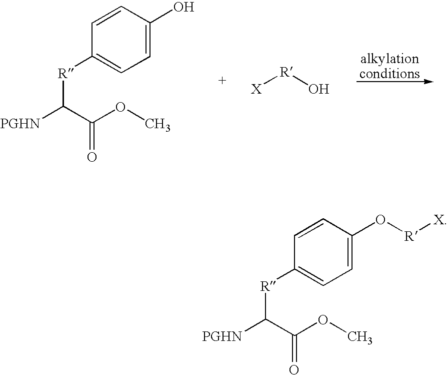

The ester may be transformed into the corresponding amide by reaction with e.g. ammonia in a suitable solvent or mixture of solvents such as e.g. water or N,N-dimethylformamide.

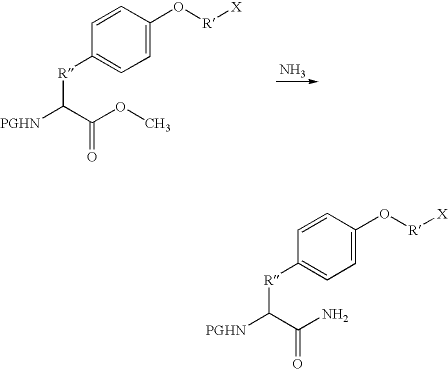

The removal of all protective groups may be performed in one or several steps by methods as described in the literature (e.g. T. W. Greene, P. G. M. Wuts, Protective groups in organic synthesis, 2^{nd} ed., 1991 John Wiley & Sons, Inc. New York)

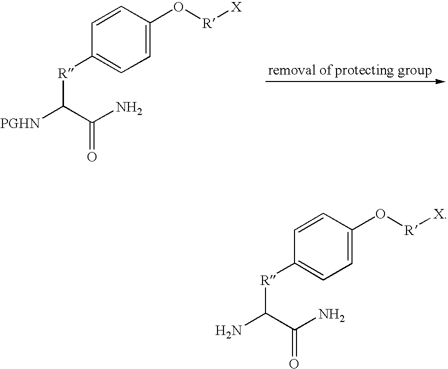

As defined in General Method (B)

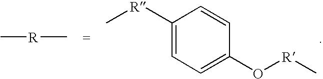

### General Method (C):

A compound of the general formula

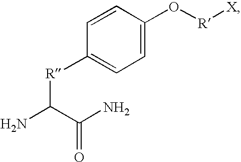

wherein R′ and R″ are defined as above, may be prepared from a suitable amino acid methyl ester which is protected at the alpha-amino group by a suitable protecting group PG, as and described in the literature, e.g. in T. W. Greene, P. G. M. Wuts, Protective groups in organic synthesis, 2^{nd} ed., 1991 John Wiley & Sons, Inc. New York)

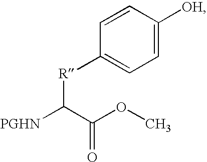

by an alkylation of the aromatic hydroxyl group, using an suitable alkylation reagent

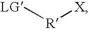

in which the anion of LG′ is a suitable leaving group such as halogenide or sulfonate and X may or may not be protected by a suitable protective group as described in the literature, e.g. in T. W. Greene, P. G. M. Wuts, Protective groups in organic synthesis, 2^{nd} ed., 1991 John Wiley & Sons, Inc. New York. The reaction may take place under basic conditions, applying bases such as e.g. potassium carbonate, diazabicylo[5,4,0]undec-5-ene, or tert-butyltetramethyluanidine at a suitable temperature, typically between −78° C. and 200° C.

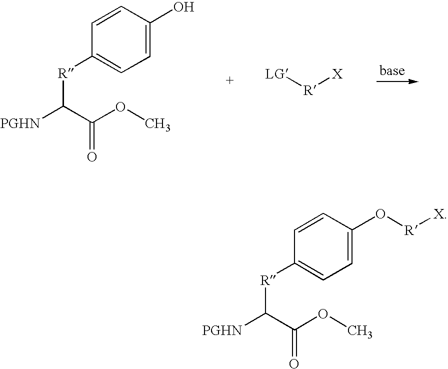

The ester may be transformed into the corresponding amide by reaction with e.g. ammonia in a suitable solvent or mixture of solvents such as e.g. water or N,N-dimethylformamide.

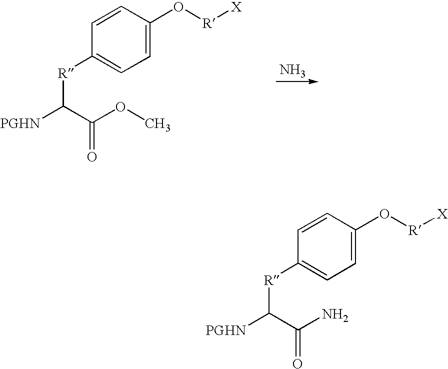

The removal of all protective groups may be performed in one or several steps by methods as described in the literature, e.g. in T. W. Greene, P. G. M. Wuts, Protective groups in organic synthesis, 2^{nd} ed., 1991 John Wiley & Sons, Inc. New York

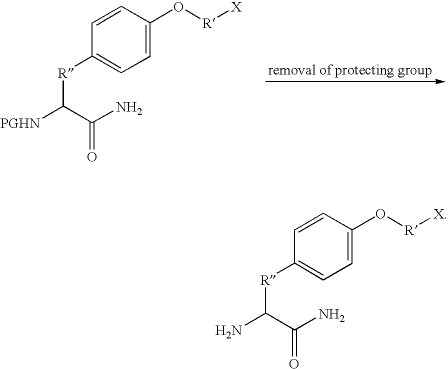

As defined in General Method (C)

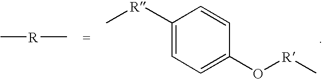

### General Method (D):

A compound of the general formula

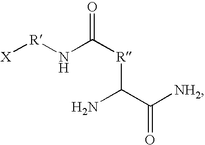

wherein R′ and R″ are defined as above, may be prepared from a suitable acid which is protected at the alpha-amino group by a suitable protecting group PG, as described in the literature, e.g. in T. W. Greene, P. G. M. Wuts, Protective groups in organic synthesis, 2^{nd} ed., 1991 John Wiley & Sons, Inc. New York

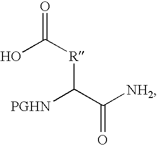

by reaction with a suitable primary or secondary amine, in which X may or may not be protected by a suitable protecting group, using acylation conditions known to a person skilled in the art e.g. a coupling reagent such as e.g. 1-hydroxybenzotriazole, 3,4-dihydro-3-hydroxybenzotriazin-4-one or 7-azabenzotriazole in combination with e.g. a carbodiimide such as e.g. diisopropylcarbodiimide or 1-(3-dimethylaminopropyl)-3-ethylcarbodiimide hydrochloride in the presence or absence of a suitable base such as e.g. triethylamine or ethyldiisopropylamine to form an amide

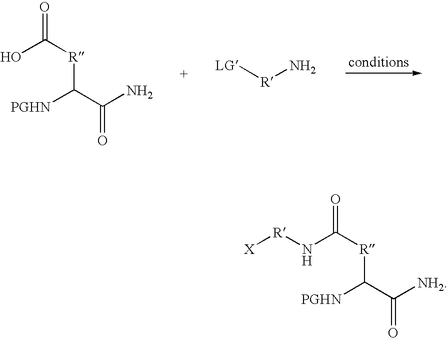

The removal of all protective groups may be performed in one or several steps as described in the literature, T. W. Greene, P. G. M. Wuts, Protective groups in organic synthesis, 2^{nd} ed., 1991 John Wiley & Sons, Inc. New York

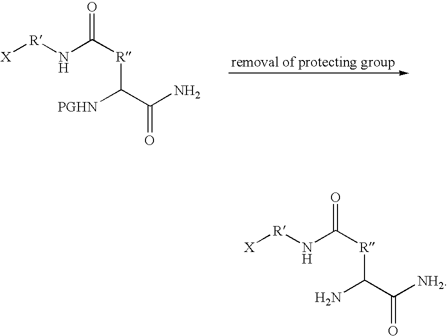

As defined in General Method (D)

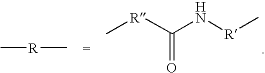

General Method (E): Synthesis of Ketogroup-Containing Amino Acid Amides from Cysteine

A conveniently N-protected cysteine derivative (for instance an ester, N-(2,4-dimethoxybenzyl)amide or N-bis(cyclopropyl)methyl amide) or conveniently N-protected cysteine amide is treated with a carbonyl-group-containing alkylating agent (R⁵¹CO(CH₂)ₙLG″, LG″=leaving group for nucleophilic displacement selected from halogen, sulfonate (—O—SO₂—R⁵¹), dialkylsulfonium, phenyliodonium, or hydroxy, wherein R⁵¹ represents C₁₋₆alkyl, partially or completely fluorinated C₁₋₆alkyl, or aryl, optionally substituted with alkyl, halogen, nitro, cyano, or acetamido, and R⁵⁰ represents hydrogen, alkyl, aryl, or heteroaryl, said aryl or heteroaryl being optionally substituted once or several times with C₁₋₆alkoxy, hydroxy, halogen, cyano, acyl, alkyl, or nitro, under suitable reaction conditions to yield an S-alkylated cysteine derivative. This derivative is converted into an amino acid amide by conversion of the acid derivative into an amide and deprotection of the alpha-amino group. Suitable N-protecting groups are for instance trityl, phthaloyl, or alkoxycarbonyl groups, such as tert-butyloxycarbonyl

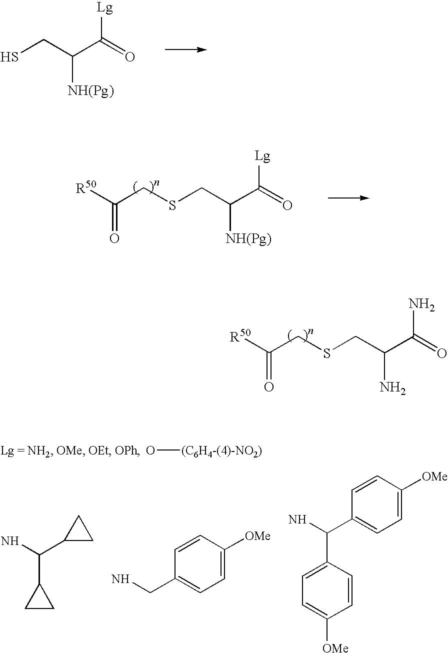

wherein n represents an integer from 1 to 10.
General Method (F): Synthesis of Ketogroup-Containing Amino Acid Amides from Aspartic or Glutamic Acid
Aspartic or glutamic acids can be selectively protected by treatment of an N-alkoxycarbonyl derivative with formaldehyde, to yield cyclic esters as shown below:

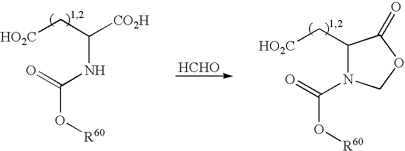

These derivatives, in which R⁶⁰ represents tert-butyl, benzyl, 2-chlorobenzyl, allyl, 2-(trimethylsilyl)ethyl, 2,2,2-trichloroethyl, or benzhydryl, can be converted to protected, ketone-containing amino acid derivatives by activation of the carboxylic acid (LvG representing halogen, aryloxy, or heteroaryloxy) and reaction with a carbon nucleophile R⁸⁰—M¹, in which R⁸⁰ represents alkyl, aryl, or heteroaryl, said aryl or heteroaryl being optionally substituted once or several times with C₁₋₆alkoxy, hydroxy, halogen, cyano, acyl, alkyl, or nitro, and in which M¹ represents an alkali metal, Mg, Zn, Ti, Zr, Mn, Cu, Ce, or Ca, optionally in the presence of a suitable catalyst. Reaction of the product with ammonia and deprotection will yield the desired amino acid amide

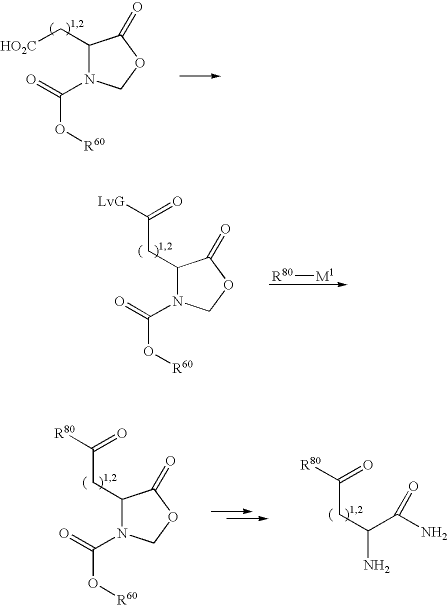

Similarly, reaction of N-alkoxycarbonyl pyroglutamic acid esters, in which R⁷⁰ represents tertbutyl, benzyl, 2-chlorobenzyl, allyl, 2-(trimethylsilyl)ethyl, 2,2,2-trichloroethyl, or benzhydryl, and R⁸⁰ represents lower alkyl, with nucleophilic carbon reagents can yield protected, ketogroup-containing amino acid derivatives. Reaction of the product with ammonia and deprotection will yield the desired amino acid amide:

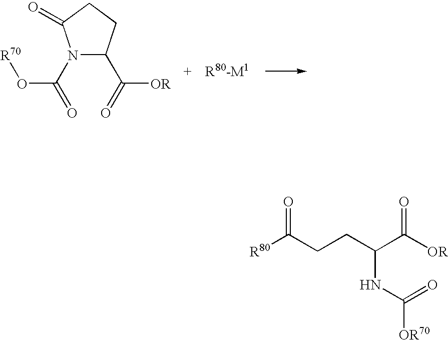

Similarly, suitably N-protected glutamic acid diesters as those shown below, in which R⁹⁰ represents lower alkyl, can be selectively acylated at carbon to yield, after hydrolysis and de-carboxylation, protected derivatives of keto-group-containing amino acids, which can be converted into amino acid amides using standard procedures

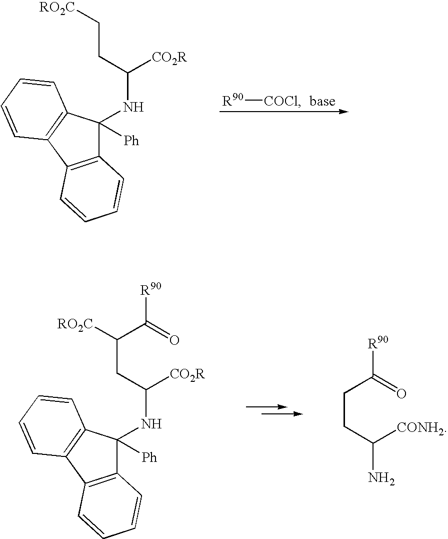

### General Method (G)

A compound of the general formula

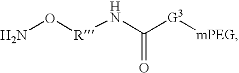

wherein R′″ represents C₁₋₁₅alkylene, C₂₋₁₅alkenylene, C₂₋₁₅alkynylene, C₁₋₁₅heteroalkylene, C₂₋₁₅heteroalkenylene, C₂₋₁₅heteroalkynylene, wherein one or more homocyclic aromatic compound biradical or heterocyclic compound biradical may be inserted and wherein G³ is a bond or a linker, may be prepared from a suitable protected primary or secondary amine

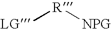

in which PG may be a suitable protection group, as described in the literature, e.g. in T. W. Greene, P. G. M. Wuts, Protective groups in organic synthesis, 2^{nd} ed., 1991 John Wiley & Sons, Inc. New York, and wherein the anion of LG′″ is a leaving group, such as e.g. halogenide or sulfonate.
This amine is reacted with a suitable protected hydroxylamine

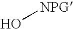

wherein PG′ is a protecting group, which is chosen in a way that PG can be removed from an amine without removal of PG′ from the hydroxylamine. Examples for that can be found in the literature, e.g. in T. W. Greene, P. G. M. Wuts, Protective groups in organic synthesis, 2^{nd} ed., 1991 John Wiley & Sons, Inc. New York.

The two components are reacted under basic conditions such as e.g. sodium hydride at a suitable temperature such as e.g −78° C. to 200° C.

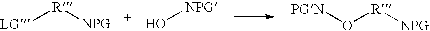

The protecting group of the amine may be removed selectively with a method described in the literature

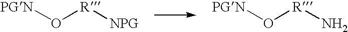

The amine may be acylated with a suitable acid and a coupling reagent such as e.g. 1-hydroxybenzotriazole, 3,4-dihydro-3-hydroxybenzotriazin-4-one or 7-azabenzotriazole in combination with e.g. a carbodiimide such as e.g. diisopropylcarbodiimide or 1-(3-dimethylaminopropyl)-3-ethylcarbodiimide hydrochloride in the presence or absence of a suitable base such as e.g. triethylamine or ethyldiisopropylamine, or with an active ester of a suitable acid such as e.g. 2,5-pyrrolidin-1-yl-ester, to give an amide.

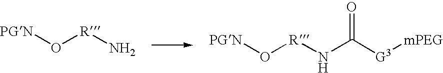

Finally, the protecting group of the hydroxylamine may be removed by a method described in the literature, e.g. in T. W. Greene, P. G. M. Wuts, Protective groups in organic synthesis, 2^{nd} ed., 1991 John Wiley & Sons, Inc. New York

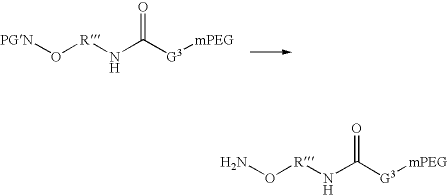

### General Method (H)

### A Compound of the General Formula

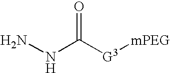

wherein G³ is a bond or a linker may be prepared from a suitable ester, in which R^{IV} is C₁₋₁₀alkyl in a suitable solvent such as ethanol by addition of hydrazine hydrate.

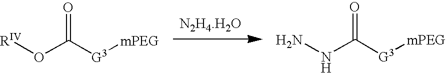

### General Method (J) Transacylation Reaction

At a suitable temperature such as e.g. 5-50° C. or room temperature, a solution of GH-XX-Ala, (final concentration 0.01-10 mM) and the nucleophile in question (final concentration 10 mM-2M) is dissolved or suspended in water containing low concentrations of EDTA.

Organic solvents may be added to improve the solubility of the reactants. The mixture may be buffered to a suitable pH-value such as e.g. between pH 1 and pH 14, between e.g. between pH 3.5 and pH 9, between pH 6 and pH 8.5, with a suitable buffer such as e.g. phosphate buffer or HEPES, or the pH can be maintained by addition of base or acid. Carboxypeptidase Y is added to the said mixture of peptide and nucleophile. The reaction may be stopped after a suitable time e.g. between 5 min and 10 days, by changing temperature or pH-value, by adding organic solvents, by addition of an inhibitor of CPY, such as e.g. phenylmethane sulfonyl fluoride, or by dialysis or gel filtration.

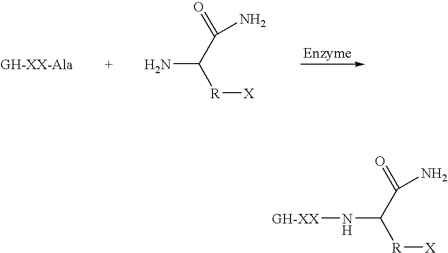

### General Method (K) Oxime Formation

An oxime moiety may be formed by dissolving the transacylated peptide in question, in which Rv may be a substituted or undsubstituted aromatic ring, a substituted or an unsubstituted heteroaromatic ring, hydrogen, or C₁₋₁₀alkyl, in water. Organic solvents may be added to increase solubility. The solution is buffered to a suitable pH-value such as e.g. between pH 0 and pH 14, between pH 3 and pH 6, or pH 5 and kept at a suitable temperature such as e.g. 0-60° C. The hydroxylamine in question is added, and oxime moiety is formed according to the reaction scheme below

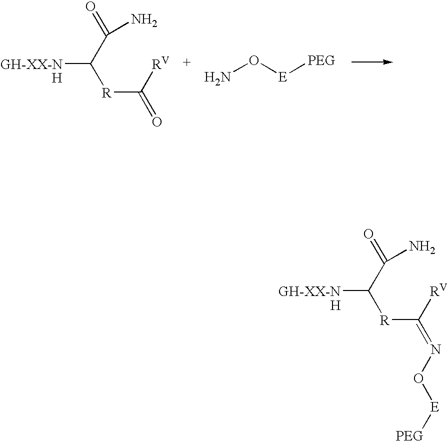

General method (L) Hydrazone Formation

### Hydrazone Formation (I)

An hydrazone moiety is formed by dissolving the transacylated peptide in question, in which R^{VI} may be a substituted or undsubstituted aromatic ring, a substituted or an unsubstituted heteroaromatic ring, hydrogen, or C₁₋₁₀alkyl, in water. The solution is buffered to a suitable pH-value such as e.g. between pH 2 and pH 14 or between pH 0 and pH 4 and kept at a suitable temperature such as e.g. 0-60° C. The hydrazide in question is added, whereby the hydrazone is formed

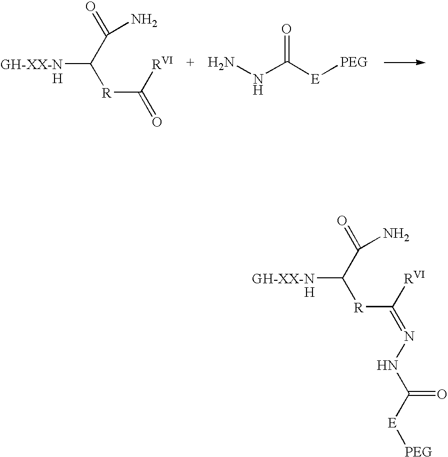

### General Method (M) Isoxazole Formation

An isoxazole can be formed by reaction between a nitril-oxide and an alkyne. The nitril-oxide is formed by addition of a suitable oxidation-reagent such as e.g. bleach to an excess of a suitable oxime. A solution of an excess of the freshly formed nitrile-oxide may be added to the peptide in question.

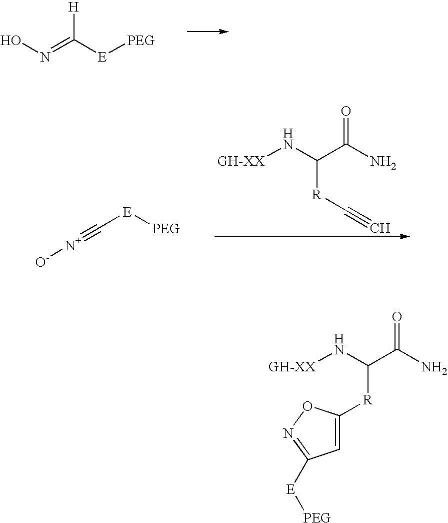

### General Method (N) Triazole Formation

A triazole can be formed by reaction between an azide which is attached to PEG and an alkyne, which is attached to the growth hormone in question, in the presence of Cu(I)-ions in a suitable solvent such as water or a mixture of water and an organic solvent such as e.g. acetonitrile. The triazole may be formed in two possible regioisomers.

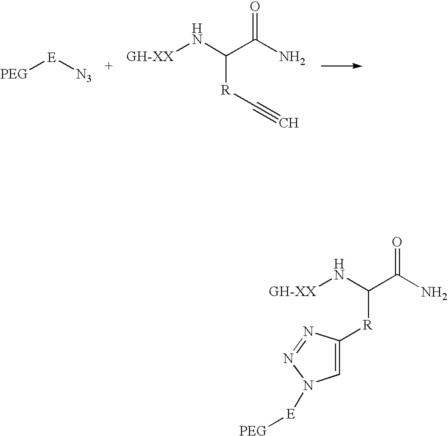

### General Method (O) Triazole Formation

A triazole can be formed by reaction between an alkyne which is attached to PEG and an azide, which is attached to the growth hormone in question, in the presence of Cu(I)-ions in a suitable solvent such as water or a mixture of water and an organic solvent such as e.g. acetonitrile. The triazole may be formed in two possible regioisomers.

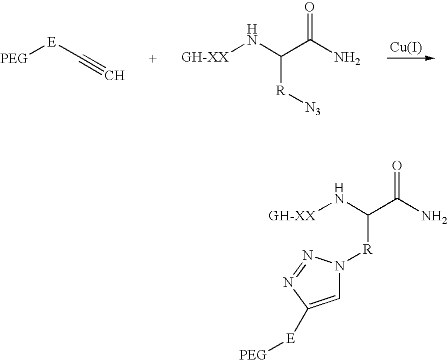

### General Method (P) Amide Formation

An amide can be regioselectively formed by reaction of an azide, which is covalently attached to growth hormone with an ester, containing a triphenylphosphine-moiety as it is described in e.g. Tetrahedron Lett. 2003, 44, 4515-4518.

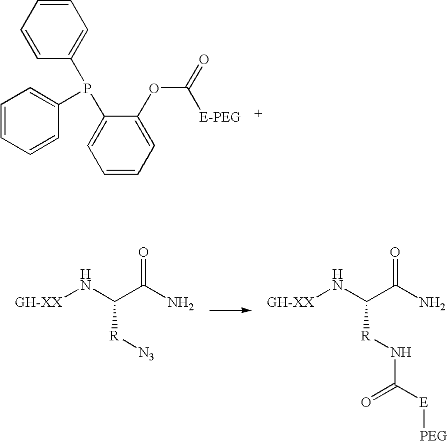

### General Method (Q) Amide Formation

An amide can be regioselectively formed by reaction of an azide, which is covalently attached to growth hormone with a thioester, containing a diphenylphosphine-moiety as it is described in e.g. J. Org. Chem. 2002, 67, 4993-4996.

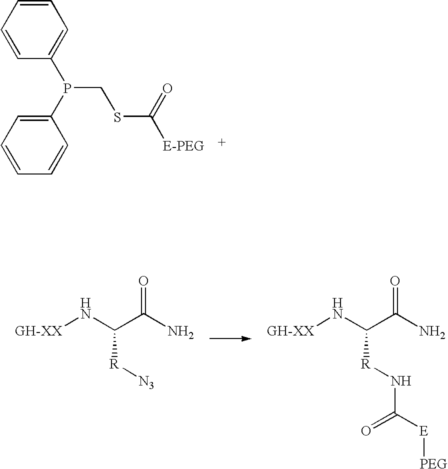

### General Method (R) Arylalkyne Formation

An arylalkyne can be formed by reaction between an alkyne, which is covalently attached to a growth hormone and a haloaryl compound in the presence of a palladium catalyst, which is water-soluable, as described in e.g. Bioconjugate Chemistry, 2004, 15, 231-234. The haloaryl compound may be exchanged with the corresponding aryl trifluorosulfonate.

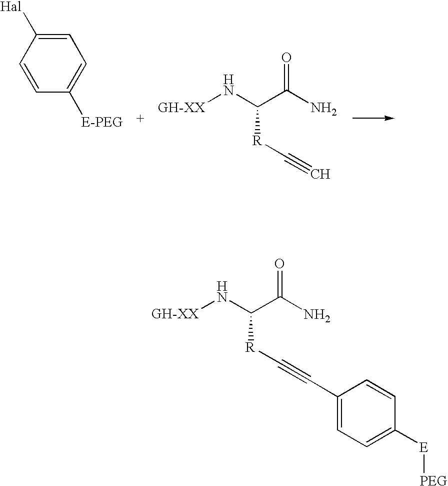

### General Method (S) Arylalkyne Formation

An arylalkyne might be formed by reaction between a haloaryl-moiety, which is covalently attached to a growth hormone and an alkyne in the presence of a palladium catalyst, which is water-soluable, as described in e.g. Bioconjugate Chemistry, 2004, 15, 231-234. Instead of the haloaryl-moiety a trifluorosulfonyloxyaryl-moiety, which is attached to a peptide may be used as well.

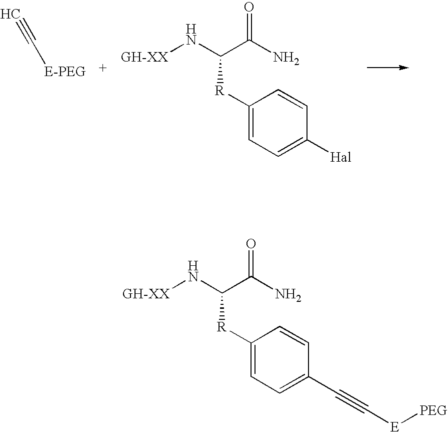

### General Method (T)

A compound of the general formula

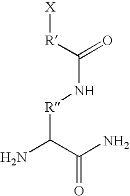

wherein R′ and R″ are as defined above may be prepared from a suitable amino acid, which is protected at the alpha-amino group, with an acid-labile protecting group PG¹ such as e.g. BOC or trityl, and which is protected at the omega-amino group with a base-labile protecting group PG² such as e.g. Fmoc. The acid may be attached to a Rink-amide resin using standard coupling conditions known to a person skilled in the art, such as e.g. use of a carbodiimide e.g. diisopropylcarbodiimide in the presence or absence of a reagent such as e.g. 1-hydroxybenzotriazole, 1-hydroxy-7-azabenzotriazole or 3,4-dihydro-3-hydroxy-4-oxo-1,2,3-benzotriazin and in the presence or absence of a base such as e.g. triethylamine or ethyldiisopropylamine. The protecting group at the omega-amine PG², may be removed under basic conditions described for the particular protecting group in the literature such as e.g. T. W. Greene, P. G. M. Wuts, Protective groups in organic synthesis, 2^{nd} ed., 1991 John Wiley & Sons, Inc. New York.

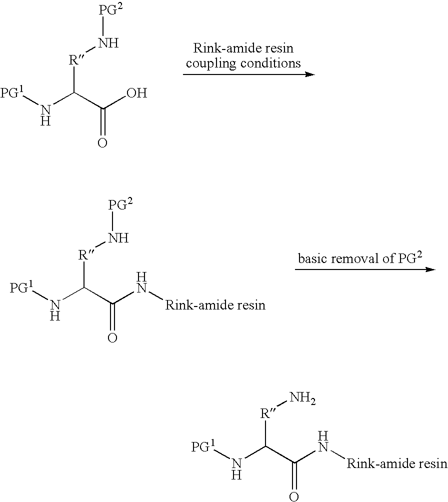

An acid can be attached to the omega amino moiety using standard coupling conditions, such as e.g. use of a carbodiimide e.g. diisopropylcarbodiimide in the presence or absence of a reagent such as e.g. 1-hydroxybenzotriazole, 1-hydroxy-7-azabenzotriazole or 3,4-dihydro-3-hydroxy-4-oxo-1,2,3-benzotriazin and in the presence or absence of a base such as e.g. triethylamine or ethyldiisopropylamine. The intermediate may be cleaved from the solid support under acidic conditions such as e.g. trifluoroacetic acid or a 20-70% solution of trifluoroacetic acid in dichloromethane to give the desired aminamide.

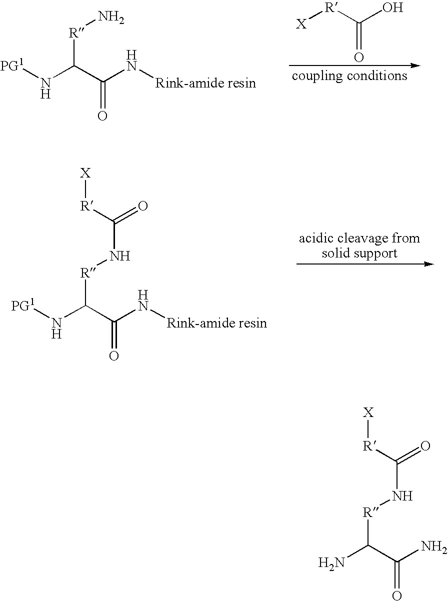

### General Method (U)

A compound of the general formula

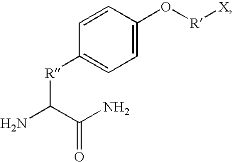

wherein R′ and R″ are defined as above, may be prepared from a suitable amino acid, which is protected with an acid labile protecting group PG¹, such as e.g. Boc or trityl, which is reacted with an excess of ammonia in the presence of a coupling reagent, such as e.g. a carbodiimide e.g. diisopropylcarbodiimide in the presence or absence of a reagent such as e.g. 1-hydroxybenzotriazole, 1-hydroxy-7-azabenzotriazole or 3,4-dihydro-3-hydroxy-4-oxo-1,2,3-benzotriazin.

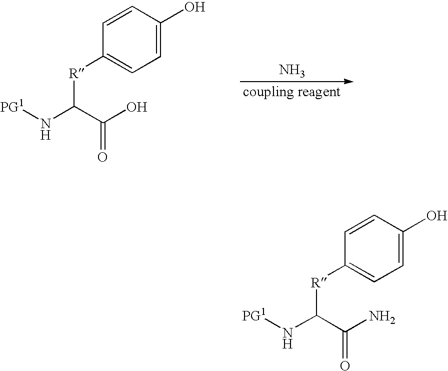

The phenolic hydroxyl group may be alkylated with a suitable halogenide or sulfonate, in which Ra is any suitable substituted alkyl or aryl radical, in the presence of a suitable base such as e.g. potassium carbonate or tetramethylguanidine. The protecting group PG¹ may be removed from the alpha amino acid under acidic conditions and described in the literature for the particular protecting group chosen e.g. in T. W. Greene, P. G. M. Wuts, Protective groups in organic synthesis, 2^{nd} ed., 1991 John Wiley & Sons, Inc. New York, to give the desired amino amide.

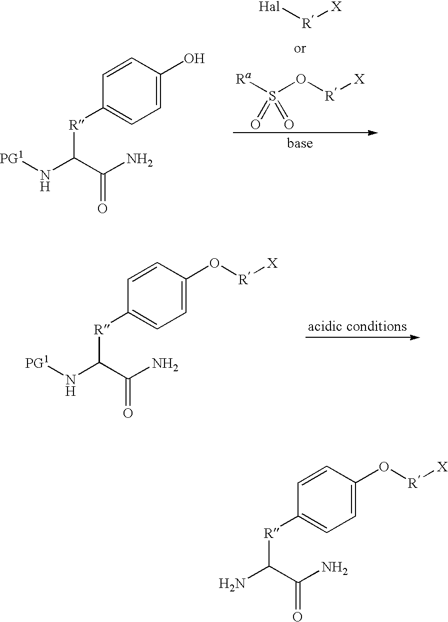

### General Method (V) PEG-Reagent

### A Reagent of the General Formula

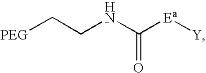

in which

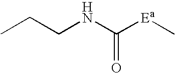

is E, as defined above,
may be prepared from a suitable acid, which may be activated by reaction with a suitable reagent or a combination of reagents, such as e.g. 2-succinimido-1,1,3,3-tetramethyluronium tetrafluoroborate (TSTU) in a suitable solvent such as e.g. N,N-dimethylformamide. The activated acid e.g. the obtained 2,5-dioxopyrrodin-1-yl ester of said acid may be reacted with commercially available PEG-reagents, which are functionalized with a primary amine, optionally in the presence of a suitable base such as e.g. ethyldiisopropylamine or triethylamine.

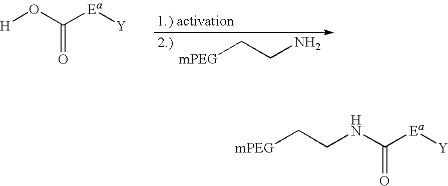

### General Method (W) PEG-Reagent

A reagent of the general formula

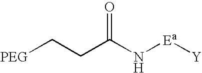

in which

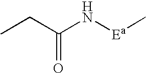

### is E,

may be prepared from a commercially available succinimidyl ester

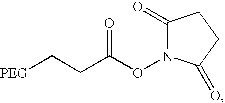

which is reacted with a suitable amine

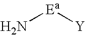

optionally in the presence of a base such as e.g. an amine-base, such as e.g. triethylamine or ethyldiisopropylamine.

### Example 1 N-(4-Aminoxybutyl)-4-(mPEG20000-yloxy)butanolyamide

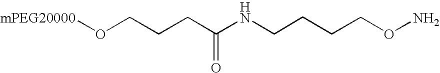

### Step 1

2-(4-(tert-Butoxycarbonylaminoxy)butyl)isoindole-1,3-dione

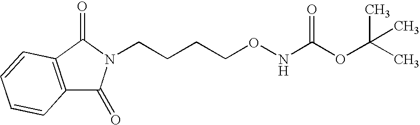

To a mixture of commercially available N-(4-bromobutyl)phthalimide (2.82 g, 10 mmol) and N-Boc-hydroxylamine (2.08 g, 15.6 mmol) was added acetonitrile (2 ml) and successively 1,8-diazabicyclo[5.4.0]undec-7-ene (2.25 ml, 15 mmol). The reaction mixture was stirred at room temperature for 30 min and then at 50° C. for 2 days. It was diluted with a mixture of water (30 ml) and 1 N hydrochloric acid (20 ml). It was extracted with ethyl acetate (2×100 ml). The organic phase was washed with brine (50 ml) and was dried over magnesium sulphate. The crude product was purified by chromatography on silica (60 g), using a gradient of heptane/ethyl acetate 1:0 to 0:1 as eluent to give 2.08 g of 2-(4-(tert-butoxycarbonylaminoxy)butyl)isoindole-1,3-dione.

### Step 2:

N-(4-aminobutoxy)carbamic acid tert-butyl ester

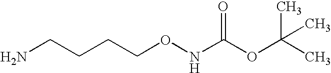

Hydrazine hydrate (1.0 ml, 20 mmol) was added to a solution of 2-(4-(tert-butoxycarbonylaminoxy)butyl)isoindole-1,3-dione (2.08 g, 6.22 mmol) in ethanol (8.0 ml). The reaction mixture was stirred at 80° C. for 65 h. The solvent was removed in vacuo. The residue was dissolved in toluene (10 ml) and the solvent was removed in vacuo. The residue was suspended in 1 N hydrochloric acid (10 ml). The precipitation was removed by filtration and was washed with water (2 ml). The filtrate and the wash-liquids were combined and made basic with potassium carbonate. The solution was extracted with dichloromethane (4×20 ml). The organic layer was dried over magnesium sulphate. The solvent was removed in vacuo to give 0.39 g of N-(4-aminobutoxy)carbamic acid tert-butyl ester. Potassium carbonate (3 g) was added to the aqueous phase, which was extracted with dichloromethane (3×20 ml). These combined organic layers were dried over magnesium sulphate. The solvent was removed in vacuo to give another 0.39 g of N-(4-aminobutoxy)carbamic acid tert-butyl ester.

### Step 3:

N-(4-(4-(m P EG20000-yloxy)butanolyamino)butoxy)carbamic acid tert-butyl ester

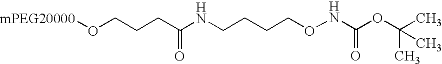

The commercially available N-hydroxysuccinimide ester of mPEG2000-yloxybutanoic acid (Nektar “mPEG-SBA”, # 2M450P01, 3 g, 0.15 mmol) was dissolved in dichloromethane (25 ml). N-(4-Aminobutoxy)carbamic acid tert-butyl ester (0.12 g, 0.59 mmol) was added. The reaction mixture was shaken at room temperature. Diethyl ether was added until a precipitation was obtained. The precipitation was isolated by filtration. The material was dried in vacuo to yield 2.39 g of N-(4-(4-(mPEG20000-yl)butanolyamino)butoxy)carbamic acid tert-butyl ester.

### Step 4:

N-(4-Aminoxybutyl)-4-(m P EG20000-yloxy)butanolyamide

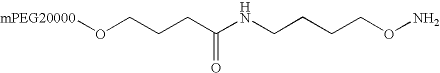

Trifluoroacetic acid (20 ml) was added to a solution of N-(4-(4-(mPEG20000-yl)butanolyamino)butoxy)carbamic acid tert-butyl ester (2.39 g, 0.12 mmol) in dichloromethane (20 ml). The reaction mixture was shaken for 30 min. Diethyl ether (100 ml) was added. The formed precipitation was isolated by filtration. It was washed with diethyl ether (2×100 ml) and dried in vacuo to give 1.96 g of N-(4-aminoxybutyl)-4-(mPEG20000-yl)butanolyamide

### Example 2 (2S)-2-(N-(hGHyl)leucinylamino)-3-(4-((1-(10-(N-(mPEG20000-yl)carbamoyl)decyl)triazol-4-yl)methoxy)phenyl)propionamide

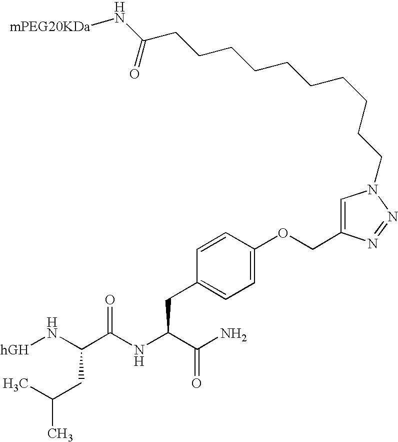

### Step 1:

[(S)-1-Carbamoyl-2-(4-hydroxyphenyl)ethyl]-carbamic acid tert-butyl ester

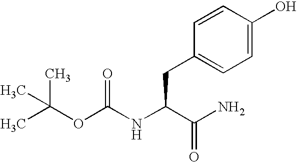

Di-tert-butyl dicarbonate (15 g, 69 mmol) was added to a solution of the hydrochloride salt of tyrosine amide (15 g, 69 mmol) in dioxane (140 ml) and a 1 N aqueous solution of sodium hydroxide (140 ml). The reaction mixture was stirred for 16 h at room temperature. It was diluted with a 10% aqueous solution of sodium hydrogensulphate (200 ml) and extracted with ethyl acetate (3×200 ml). The combined organic layers were washed with a saturated aqueous solution of sodium hydrogencarbonate (100 ml) and dried over magnesium sulphate. The solvent was removed in vacuo. The crude product was purified by flash chromatography on silica (400 g), using a mixture of dichloromethane/methanol (10:1) to give 8.17 g of [(S)-1-carbamoyl-2-(4-hydroxyphenyl)ethyl]-carbamic acid tert-butyl ester.

MS: m/z=303 (M+Na)⁺.

¹H-NMR (DMSO-d₆): δ 1.31 (s 9H); 2.80 (dd, 1H); 2.83 (dd, 1H); 4.00 (m, 1H); 6.62 (d, 2 H); 6.70 (d, 1H); 6.97 (br, 1H); 7.03 (d, 2H); 7.31 (br, 1H); 9.14 (s, 1H).

### Step 2:

[(S)-1-Carbamoyl-2-(4-(prop-2-ynyloxy)phenyl)ethyl]carbamic acid tert-butyl ester

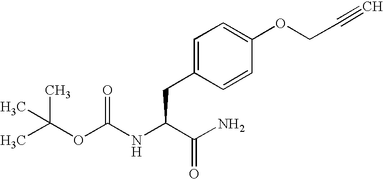

A mixture of [(S)-1-carbamoyl-2-(4-hydroxyphenyl)ethyl]-carbamic acid tert-butyl ester (1.0 g, 3.57 mmol), tetrabutylammonium iodide (65 mg, 0.17 mmol), potassium carbonate (3.94 g, 29 mmol), propargyl bromide (0.38 ml, 4.28 mmol) and N,N-dimethylformamide (15 ml) was heated to 60° C. for 16 h. It was cooled to room temperature, diluted with water (30 ml) and acidified with a 10% aqueous solution of sodium hydrogensulphate. The mixture was extracted with ethyl acetate (2×100 ml). The combined organic layers were washed with a saturated aqueous solution of sodium hydrogencarbonate (200 ml) and dried over magnesium sulphate. The solvent was removed in vacuo. The crude product was purified by flash chromatography on silica (100 g), using a mixture of dichloromethane/methanol (10:1) as eluent, to give 998 mg of [(S)-1-carbamoyl-2-(4-(prop-2-ynyloxy)phenyl)ethyl]carbamic acid tert-butyl ester.

MS: m/z=341 (M+Na)⁺.

¹H-NMR (DMSO-d₆) δ 1.31 (s, 9H); 2.50 (s, 1H); 2.67 (dd, 1H); 2.91 (dd, 1H); 4.03 (m, 1H); 4.74 (s, 2H); 6.77 (d, 1H); 6.86 (d, 2H); 6.99 (s, 1H), 7.17 (d, 2H); 7.35 (s, 1H).

### Step 3:

(2S)-2-Amino-3-(4-(prop-2-ynyloxy)phenyl)propionamide

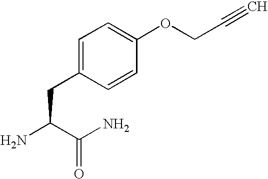

Trifluoroacetic acid (10 ml) was added to a solution of [(S)-1-carbamoyl-2-(4-(prop-2-ynyloxy)phenyl)ethyl]carbamic acid tert-butyl ester (998 mg, 3.13 mmol) in dichloromethane (10 ml). The reaction mixture was stirred for 1.5 h at room temperature. The solvent was removed. The residue was dissolved in dichloromethane (30 ml). The solvent was removed. The latter procedure was repeated twice to give 1.53 g of the trifluoroacetate salt of (2S)-2-amino-3-(4-(prop-2-ynyloxy)phenyl)propionamide.

HPLC (method O₂-B4-4): R_{f}=5.62 min.

MS: m/z=219 (M+1)⁺.

¹H-NMR (CDCl₃) δ 2.51 (s, 1H); 3.02 (m, 2H); 3.90 (m, 1H); 4.78 (s, 2H); 6.95 (d, 2H); 7.20 (d, 2H); 7.56 (s, 1H); 7.87 (s, 1H); 8.10 (br, 3H).

### Step 4:

11-Azidoundecanoic acid 2,5-dioxopyrroldin-1-yl ester

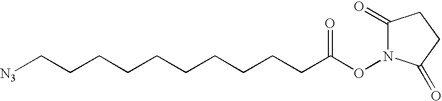

N,N,N′,N-Tetramethyl-O-(N-succinimidyl)uranium tetrafluoroborate (1.32 g, 4.40 mmol) was added to a solution of 11-azidoundecanoic acid (1.00 g, 4.40 mmol) and triethylamine (0.61 ml, 4.40 mmol) in N,N-dimethylformamide (10 ml). The reaction mixture was stirred for 2 h at room temperature. It was diluted with ethyl acetate (50 ml) and washed with water (3×50 ml). The organic phase was dried over sodium sulphate. The solvent was removed in vacuo to give 1.40 g of crude 11-azidoundecanoic acid 2,5-dioxopyrroldin-1-yl ester, which was used in the next steps without further purification.

MS: m/z=347 [M+Na⁺]

¹H-NMR (CDCl₃): δ 1.35 (m, 12H); 1.60 (quintett, 2H); 1.75 (quintett, 2H); 2.60 (t, 2H); 1.85 (m, 4H); 3.25 (t, 2H).

### Step 5:

11-AzidoundecanoylaminomPEG20 kDa

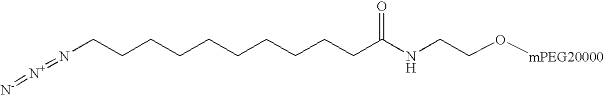

A solution of 11-azidoundecanoic acid 2,5-dioxopyrroldin-1-yl ester (227 mg, 0.7 mmol) was added to a solution of commercially available mPEG20000DA-amine (Nektar 2M2U0P01, 5.00 g, 0.25 mmol) and triethylamine (0.174 ml, 1.25 mmol) in dichloromethane (50 ml). The reaction mixture was stirred at room temperature for 16 h. Ether (800 ml) was added. The formed precipitation was isolated by filtration and washed with ether (2×100 ml). It was dried in vacuo to give 4.58 g of 11-azidoundecanoylaminomPEG20 kDa.

### Step 6:

(S)-3-(4-(propargyloxy)phenyl)-2-((S)-2-hGHylleucinylamino)propionamide

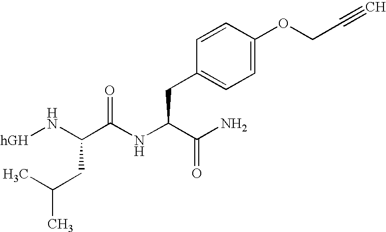

At room temperature a aqueous solution of CPY (200 U/ml, 1 U, 0.005 ml) was added to a solution of hGH-Leu-Ala (1 mg, 45 nmol) and the trifluoroacetate salt of (2S)-2-Amino-3-(4-(prop-2-ynyloxy)phenyl)propionamide (5.2 mg, 0.157 mmol) in a buffer (0.090 ml) consisting of 0.25 M HEPES and 5 mM EDTA, which was adjusted with an aqueous 1 N sodium hydroxide solution to pH 8.02. After 4.5 h, the reaction mixture was diluted with water (0.800 ml). A freshly prepared solution of phenylmethanesulfonyl fluoride (0.32 mg, 1808 nmol) in isopropanol (0.900 ml) was added.

MALDI-MS (CHCA): 11202 (M²⁺)

The products of three runs were taken into a 50 mM TRIS-buffer, which was adjusted with 1 N hydrochloric acid to pH 8.5. They were purified by chromatography on a monoQ column (1 ml), using a gradient of 0-50% of a buffer consisting of 2.0 M sodium chloride and 50 mM TRIS, which was adjusted to pH 8.5 with 1 N hydrochloric acid in a buffer of 50 mM TRIS-buffer, which was adjusted with 1 N hydrochloric acid to pH 8.5, at a flowrate of 0.5 ml/min to give (S)-3-(4-(propargyloxy)phenyl)-2-((S)-2-hGHylleucinylamino)propionamide.

MALDI (CHCA): 22454 (M⁺); 11238 (M²⁺)

### Step 7:

A solution of L-(+)-ascorbic acid (2 mg, 0.008 mmol) and 2,6-lutidine (0.003 ml) in water (0.125 ml) was added to a solution of copper sulphate pentahydrate (0.55 mg) in water (0.125 ml). The solution was kept at room temperature for 5 min. 0.25 ml of this solution was added to another solution, which had been prepared by mixing of a solution of (S)-3-(4-(propargyloxy)phenyl)-2-((S)-2-hGHylleucinylamino)propionamide (0.5 mg, 22 nmol) and 2,6-lutidine (0.001 ml) in water (0.05 ml) and a solution of 11-azidoundecanoylaminomPEG20 kDa (4.51 mg, 223 mmol) in water. The reaction mixture was kept at room temperature for 21 h. The SDS-gel showed the formation of a product with a virtual molecular weight of approx. 65 kDa, in accordance with the expectation for a PEGylated product, since it is known, that pegylated compounds show in SDS-gels higher molecular weights than calculated.

### Example 3

### (S)-2-((hGHleucinyl)amino)-6-(4-(1-(4-(4-(20 kDa mPE-Gyl)butanoylamino)butoxyimino)ethyl)benzoylamino)hexanoic amide

Step 1:

CPY-catalyzed transpeptidation of hGH-Leu-Ala with 4-acetyl-N-((5S)-5-amino-5-carbamoylpentyl)benzamide:

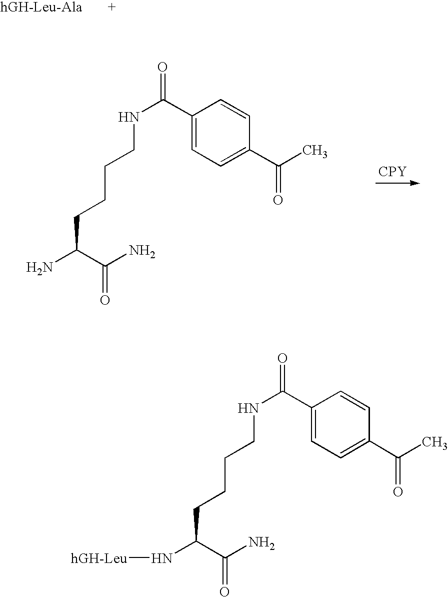

To a solution of hGH-Leu-Ala (20 mg, 0.5 mM final concentration) in H₂O:diisopropylamine (100:1 v/v, 0.6 ml), was added 4-acetyl-N-((5S)-5-amino-5-carbamoylpentyl)benzamide (127 mg, 175 mM final concentration) in solution in HEPES buffer 250 mM pH8.5 containing 5 mM EDTA (0.97 ml). The pH is adjusted to 8.2 by addition of sodium hydroxide (10M in water). The reaction volume was adjusted to 1.77 ml by addition of HEPES buffer 250 mM pH8.5 containing 5 mM EDTA. The reaction was started by addition of the enzyme (Fluka #21943) in solution in water (10 U/ml final concentration). The reaction mixture was incubated at 30° C.

The reaction was monitored by capillary electrophoresis.

CE Analysis Method:

The capillary electrophoresis was performed using a Hewlett Packard 3D CE system equipped with a diode array detector.

The fused silica capillary (Agilent) used had a total length of 64.5, an effective length of 56 cm and an ID of 50 μm. Samples were injected by pressure at 50 mbar for 4s. Separations were carried out at 30° C., under a tension of +25 kV, using phosphate buffer 50 mM pH7 as electrolyte. The analysis was monitored at 200 nm. Between runs, an acidic and a basic wash were performed: the capillary was rinsed with water for 2 min, then with phosphoric acid 0.1 M for 2 min, then with water for 2 min; this was followed by rinsing with sodium hydroxide 0.1 M for 3 min, and water for 2 min, before equilibrating the capillary with the electrolyte.

MALDI-TOF Analysis Method:

The compounds present in the reaction mixture were identified by MALDI-TOF, using α-cyano-4-hydroxy-cinnamic acid as the matrix:

m/z=11156 hGH-Leu-Ala (M+2H)²⁺

m/z=11257 (S)-2-((hGHleucinyl)amino)-5-carbamoylpentyl) 4-acetyl benzamide

(M+2H)²⁺

m/z=11120 hGH-Leu (M+2H)²⁺

When the yield of transpeptidated product reached >80%, the reaction was stopped by inactivating the enzyme with PMSF (100 mM in solution in dry isopropanol, final concentration 2 mM).

After dilution in Tris, HCl buffer 50 mM pH8.5, the excess of reagent was eliminated by ultrafiltration (Millipore Amicon Ultra 10 kD MW) followed by gel filtration on Sephadex G25 (Amersham).

The solution obtained was used in the next step without further purification.

Step 2:

Oximation with N-(4-aminoxybutyl)-4-(20 kDa mPEGyloxy)butanoic amide:

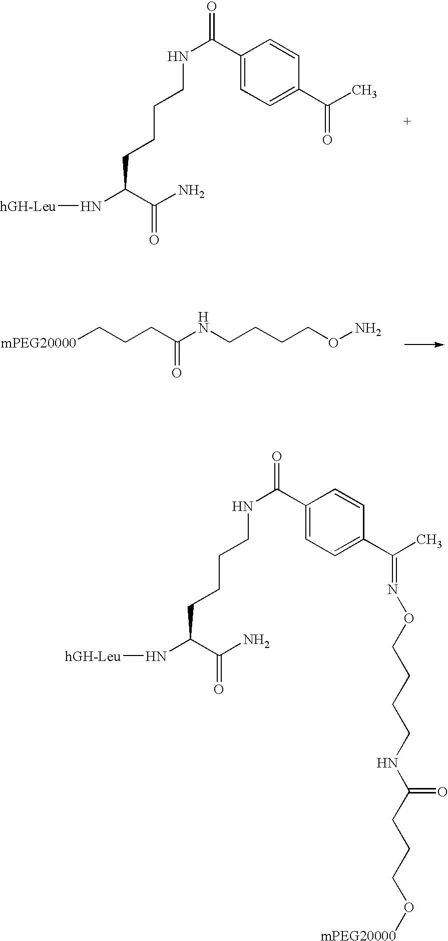

To an aliquot of the solution obtained in the first step (850 μl), containing about 16 mg of transpeptidation product (about 0.7 μmole), was added neat acetic acid to adjust the pH to 4. The mixture was then put on ice before NMP (750 μl) was added (final concentration 25% v/v). The mixture was put at ambiant temperature again, and added to (4-aminoxybutyl)-4-(20 kDa mPEGyloxy)butanoic amide (425 mg, 21 μmoles) in solution in acetate buffer 50 mM pH4 (0.9 ml). The reaction mixture was diluted twice by addition of acetate buffer 50 mM pH4.

The reaction mixture was put at 30° C. under nitrogen for 24 h.

The reaction was followed by HPLC:

HPLC Method:

The analysis was performed using an Agilent 1100 HPLC system equipped with a diode array detector. The column used was a reverse phase Vydac C18 (218TP53) 250×4.6. The elution was performed with the following eluents: A: H₂₀/TFA 0.1% and B: ACN/TFA 0.1%, with a gradient from 10 to 91% B over 27 min, with a flow of 1 ml/min. The analysis was performed at 40° C., and the detection was done at 214, 254, and 280 nm.

Rt of (S)-2-((hGHleucinyl)amino)-5-carbamoylpentyl) 4-acetyl benzamide: 19.6 min

Rt of hGH-Leu: 19.6 min

Rt of the product (S)-2-((hGHleucinyl)amino)-6-(4-(1-(4-(4-(20 kDa mPE-Gyl)butanoylamino)butoxyimino)ethyl)benzoylamino)hexanoic amide: 18.6 min

An SDS gel run after 24 h showed the formation of the pegylated derivative with an estimated yield of 60%.

### Example 4

### 4-Acetyl-N-((5S)-5-amino-5-carbamoylpentyl)benzamide

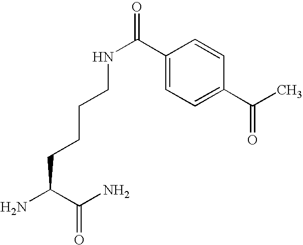

Rink-amide-resin (loading: 0.43 mmol/g, 6.66 g, 2.86 mmol) was swelled with dichloromethane (50 ml). The solvent was removed. A 20% solution of piperidine in N-methylpyrrolidinone was added (50 ml). The reactor was shaken for 20 min. The liquid was removed. The resin was washed with N-methylpyrrolidinone (3×50 ml) and dichloromethane (5×50 ml). A solution of BOC-Lys(FMOC)—OH (5.37 g, 11.5 mmol) in N-methylpyrrolidinone (50 ml) and a solution of 1-hydroxybenzotriazole (1.75 g, 11.5 mmol) in N-methylpyrrolidinone (20 ml) were added successively. Diisopropylcarbodiimide (1.79 ml, 11.5 mmol) and ethyldiisopropylamine (1.96 ml, 11.5 mmol) were added. The reactor was shaken at room temperature for 16 h. The liquid was removed. The resin was washed with N-methylpyrrolidinone (3×50 ml) and dichloromethane (3×50 ml). A solution of 4-acetylbenzoic acid (2.82 g, 11.5 mmol) in N-methylpyrrolidinone (50 ml) and a solution of 1-hydroxybenzotriazole (1.75 g, 11.5 mmol) in N-methylpyrrolidinone (20 ml) were added successively. Diisopropylcarbodiimide (1.79 ml, 11.5 mmol) and ethyldiisopropylamine (1.96 ml, 11.5 mmol) were added. The reactor was shaken at room temperature for 16 h. The resin was washed with N-methylpyrrolidinone (3×50 ml) and dichloromethane (3×50 ml). A solution of 50% of trifluoroacetic acid and 10% triisopropylsilane in dichloromethane (50 ml) was added to the resin. The reaction vessel was shaken for 1 h at room temperature. The liquid was collected. The solvent was removed in vacuo. The residue was redissolved in toluene (50 ml). The solvent was removed in vacuo.

The crude products of 6 runs of the procedure described above were combined. They were purified by HPLC-chromatography on a C₁₋₈-reversed phase column, using a gradient of 3-23% of acetonitrile in water in a 0.1% buffer of trifluoroacetic acid to afford 1.07 g of the trifluoroacetic acid salt of 4-acetyl-N-((5S)-5-amino-5-carbamoylpentyl)benzamide.

MS: m/z=292 (M+1)⁺

HPLC (method 01-B1-2): 5.23 min

### Example 5

### Cloning, Expression and Purification of hGH-Leu-Ala

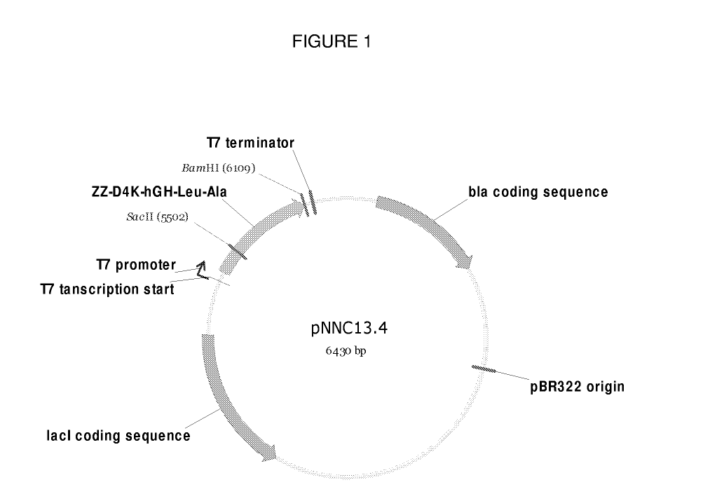

A cloning strategy based on pNNC13, a pET11a derived vector already containing Zbasic2mt-D4K-hGH has been utilized. Using pNNC13 as template and a PCR primer set flanking the Sac II and Bam HI restriction sites, a 628 bp amplicon has been generated encoding two additional amino acids (Leucine and Alanine) in the C-terminal end of hGH. This PCR amplicon was then cloned back into pNNC13 using the existing Sac II and BamHI sited to generate pNNC13.4 encoding Zbasic2mt-D4K-hGH-Leu-Ala. The integrity of the resulting clones was confirmed by DNA sequencing of the coding region. See FIG. 1.

Escherichia coli BL21 (DE3) was transformed with pET11a-Zbasic2mt-D4K-hGH-Leu-Ala. Single colony was inoculated into 100 ml LB media with 100 μg/ml Amp and grown at 37° C. until OD600 reaches 0.6. The cell culture temperature was reduced to 20° C. and the cells were induced with 1 mM IPTG for 6 hours at 20° C. The cells were harvested by centrifugation at 3000 g for 15 minutes.

The cell pellet was re-suspended in cell lysis buffer (25 mM Na₂HPO₄ 25 mM NaH₂PO₄ pH 7, 5 mM EDTA, 0.1% Triton X-100), and the cells were disrupted by cell disruption at 30 kpsi (Constant Cell Disruption Systems). The lysate was clarified by centrifugation at 10,000 g for 35 minutes and the supernatant was used for purification.

Zbasic2mt-D4K-hGH-Leu-Ala was purified on SP Sepharose FF using a step gradient elution (buffer A: 25 mM Na₂HPO₄ 25 mM NaH₂PO₄ pH 7; buffer B: 25 mM Na₂HPO₄ 25 mM NaH₂PO₄ pH 7, 1 M NaCl). The protein was subsequently cleaved using Enteropeptidase for the release of hGH-Leu-Ala. After digestion, hGH-Leu-Ala was further purified on a Butyl Sepharose 4FF column to separate the product from the Zbasic2mt-D4K domain and Enteropeptidase (buffer A: 100 mM Hepes pH 7.5, 2M NaCl; buffer B: 100 mM Hepes pH 7.5, a linear gradient was used).

As digestion is not complete, remaining Zbasic2mt-D4K-hGH-Leu-Ala has to be separated from hGH-Leu-Ala and this is done by loading the protein onto the SP Sepharose FF column again. Buffer exchange into 25 mM Na₂HPO₄ 25 mM NaH₂PO₄ pH 7 is performed on a Sephadex G-25 Medium column before purification on the SP Sepharose FF column. Zbasic2mt-D4K-hGH-Leu-Ala binds to SP Sepharose FF whereas hGH-Leu-Ala is found in the flow through.

The final product of hGH-Leu-Ala is buffer exchanged and lyophilized from 50 mM NH₄HCO₃, pH 7.8.

### Example 6

### 4-Acetyl-N-((5S)-5-amino-5-carbamoylpentyl)benzamide

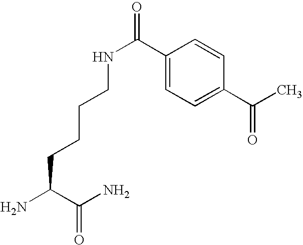

Step 1:

((S)-5-(tert-Butoxycarbonylamino)-5-(carbamoyl)pentyl)carbamic acid benzyl ester

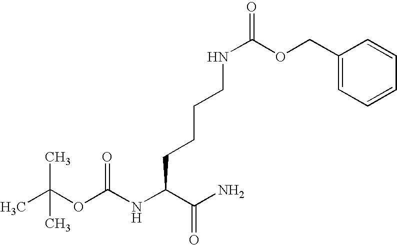

2,5-Dioxopyrrolidin-1-yl (S)-6-((benzyloxycarbonyl)amino)-2-((tertbutoxycarbonyl)amino)hexanoate (commercially available at e.g. Fluka or Bachem, 15. g, 31 mmol) was dissolved in dichloromethane (50 ml). A 25% solution of ammonia in water was added. The reaction mixture was stirred vigorously for 16 h at room temperature. The solvent was removed in vacuo to yield 21.27 g of crude ((S)-5-(tert-butoxycarbonylamino)-5-(carbamoyl)pentyl)carbamic acid benzyl ester, which was used in the next step without further purification.

¹H-NMR (DMSO-d₆): δ 1.2-1.6 (m, 6H); 1.37 (s, 9H); 2.95 (q, 2H); 3.80 (td, 1H); 5.00 (s, 2H); 6.70 (d, 1H); 6.90 (s, 1H); 7.20-7.40 (m, 7H).

MS: m/z=280.

Step 2:

((S)-5-Amino-1-(carbamoyl)pentyl)carbamic acid tert-butyl ester

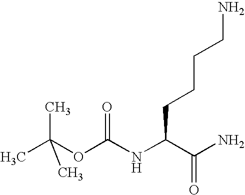

Crude ((S)-5-(tert-butoxycarbonylamino)-5-(carbamoyl)pentyl)carbamic acid benzyl ester (11.92 g, 31.41 mmol) was suspended in methanol (250 ml). Palladium on coal (50% wet) 1.67 g was added. The mixture was subjected to hydrogenation under pressure for 16 h. It was filtered through a plug of celite. The solvent was removed in vacuo to give 13.13 g of crude ((S)-5-amino-1-(carbamoyl)pentyl)carbamic acid tert-butyl ester, which was used in the next step without further purification.

¹H-NMR (DMSO-d₆): δ 1.30-1.60 (m, 6H); 1.37 (s, 9H); 2.65 (t, 2H); 3.80 (dt, 1H); 5.70 (br, 2H); 6.80 (d, 1H); 6.95 (s, 1H); 7.30 (s, 1H).

Step 3:

4-Acetylbenzoic acid 2,5-dioxopyrrolidin-1-yl ester

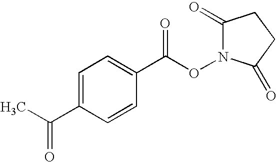

2-Succinimido-1,1,3,3-tetramethyluronium tetrafluoroborate (TSTU, 18.5 g, 60.9 mmol) was added to a solution of 4-acetylbenzoic acid (10.0 g, 60.9 mmol) and triethylamine (8.49 ml, 60.9 mmol) in N,N-dimethylformamide (50 ml). The reaction mixture was stirred for 2 h at room temperature. It was diluted with ethyl acetate (400 ml) and washed with water (3×200 ml). The organic layer was dried over sodium sulphate. The solvent was removed in vacuo to give 13.38 g of 4-acetylbenzoic acid 2,5-dioxopyrrolidin-1-yl ester.

¹H-NMR (CDCl₃): δ 2.67 (s, 3H); 2.93 (s, 4H); 8.05 (d, 2H); 8.20 (d, 2H).

MS: m/z=284 (M+Na)⁺

Step 4:

4-Acetylbenzoic acid 2,5-dioxopyrrolidin-1-yl ester (8.21 g, 31.4 mmol) was added to a suspension of crude ((S)-5-amino-1-(carbamoyl)pentyl)carbamic acid tert-butyl ester (7.71 g, 31.4 mmol) in N,N-dimethylformamide (200 ml). Ethyldiisopropylamine (16.14 ml, 94.3 mmol) was added. The reaction mixture was stirred for 3 days at room temperature. The solvent was removed in vacuo at 70° C. The residue was dissolved in dichloromethane (50 ml). Trifluoroacetic acid (50 ml) was added. The reaction mixture was stirred for 1 h at room temperature. The solvent was removed in vacuo. The residue was taken up dichloromethane (200 ml). A 10% aqueous solution of sodium hydrogen sulphate (50 ml) was added. The mixture was extracted with water (200 ml). The aqueous phase was concentrated in vacuo to approximately 60 ml. It was divided into three parts. Each part was purified by HPLC-chormatography on a C18 reversed phase column, using a gradient of 0-20% of acetonitrile in water, which was buffered with 0.1% trifluoroacetic acid, to give together 8.00 g of the trifluoroacetic salt of 4-acetyl-N-((5S)-5-amino-5-carbamoylpentyl)benzamide.

¹H-NMR (DMSO-d₆): δ1.35 (m, 2H), 1.55 (m, 2H); 1.75 (m, 2H); 2.62 (s, 3H); 3.30 (q, 2H); 3.70 (m, 1H); 7.55 (s, 1H); 7.80 (s, 1H) 7.95 (d, 2H); 8.00 (d, 2H); 8.00 (br, 3H); 8.65 (t, 1H).

MS: m/z=292.

### Example 7

### (S)-2-Amino-6-(3-(azidomethyl)benzoylamino)hexanoic amide

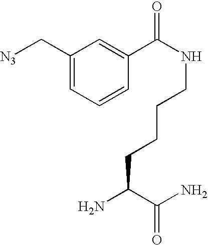

Step 1:

Methyl 3-(azidomethyl)benzoate

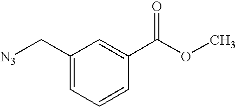

Sodium azide (5.68 g, 87 mmol) was added to a solution of methyl 3-(bromomethyl)benzoate (5.00 g, 22 mmol) in N,N-dimethylformamide (50 ml). Tetrabutylammonium iodide (81 mg, 0.22 mmol) was added. The reaction mixture was heated to 60° C. for 16 h. It was cooled to room temperature and given onto water (200 ml). This mixture was extracted with ethyl acetate (400 ml). The organic layer was washed with water (3×200 ml) and successively dried over sodium sulphate. The solvent was removed in vacuo to give 4.11 go of crude methyl 3-(azidomethyl)benzoate, which was used without further purification.

MS: m/z=192.

10 ¹H-NMR (CDCl₃): δ3.92 (s, 3H); 4.40 (s, 2H); 7.50 (m, 2H); 8.00 (m, 2H).

Step 2:

3-(Azidomethyl)benzoic acid

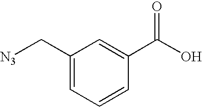

A solution of lithium hydroxide (3.81 g, 21.5 mmol) in water (25 ml) was added to a solution of crude methyl 3-(azidomethyl)benzoate (4.11 g, 21.5 mmol) in 1,4-dioxane (25 ml). Water and 1,4-dioxane was added until a clear solution was obtained. The reaction mixture was stirred for 16 h at room temperature. An 1 N aqueous solution of sodium hydroxide (100 ml) was added. The reaction mixture was washed with tert-butyl methyl ether (2×100 ml). The aqueous phase was acidified with a 10% aqueous solution of sodium hydrogensulphate. It was extracted with ethyl acetate (2×200 ml). The combined ethyl acetate phases were dried over magnesium sulphate. The solvent was removed in vacuo to give 3.68 g of crude 3-(azidomethyl)benzoic acid, which was used without further purification.

MS: m/z=150

25 ¹H-NMR (CDCl₃): δ 4.57 (s, 3H); 7.55 (m, 2H); 8.00 (m, 2H); 13.10 (br, 1H).

Step 3:

Rink Amide-resin (Novabiochem 01-64-0013, loading 0.70 mmol/g, 0.652 g, 2.7 mmol) was swelled in dichloromethane (50 ml). The solvent was removed. A 20% solution of piperidine in N,N-dimethylformamide (50 ml) was added. The mixture was shaken for 20 min at room temperature. The solvent was removed. The resin was washed with N-methylpyrrolidinone (3×50 ml) and dichloromethane (5×50 ml). A solution of Boc-Lys(FMOC)—OH (5.00 g, 10.7 mmol) in N-methylpyrrolidinone (12.5 ml) and a solution of 1-hydroxybenzotriazole (1.63 g, 10.7 mmol) were added successively to the resin. A solution of diisopropylcarbodiimide (1.67 ml, 10.7 mmol) in dichloromethane (25 ml) was added. Ethyldiisopropylamine (1.83 ml, 10.7 mmol) was added. The reaction mixture was shaken for 2 days at room temperature. The liquid was removed. The resin was washed with N-methylpyrrolidinone (3×50 ml) and dichloromethane (5×50 ml). A 20% solution of piperidine in N,N-dimethylformamide (50 ml) was added. The mixture was shaken for 20 min at room temperature. The solvent was removed. The resin was washed with N-methylpyrrolidinone (3×50 ml) and dichloromethane (5×50 ml). A solution of the crude 3-(azidomethyl)benzoic acid (1.89 g, 10.7 mmol) in N-methylpyrrolidinone (12.5 ml) and dichloromethane (12.5 ml) was added, followed by a solution of 1-hydroxybenzotriazole (1.63 g, 10.7 mmol) in N-methylpyrrolidinone (12.5 ml). A solution of diisopropylcarbodiimide (1.67 ml, 10.7 mmol) in dichloromethane (12.5 ml) was added. Ethydiisopropylamine (1.83 ml, 10.7 mmol) was added. The reaction mixture was shaken for 16 h at room temperature. The liquid was removed. The resin was washed with N-methylpyrrolidinone (3×50 ml) and dichloromethane (5×50 ml). A 50% solution of trifluoroacetic acid in dichloromethane (20 ml) was added to the resin. Triisopropylsilane (5 ml) was added. The reaction mixture was shaken for 1 h at room temperature. The liquid was collected. The resin was washed with dichloromethane (30 ml). These two latter liquids were combined. The solvent was removed in vacuo. The crude product was purified by HPLC-chromatography on a C18-reversed-phase column, using a gradient of 13-33% acetonitrile in water, which was acidified by addition of 0.1% trifluoroacetic acid to give 300 mg of the trifluoroacetate salt of (S)-2-amino-6-(3-(azidomethyl)benzoylamino)hexanoic amide.

MS: m/z=305

¹H-NMR (DMSO-d₆, trifluoroacetate salt): δ 1.37 (m, 2H); 1.55 (m, 2H); 1.77 (m, 2H); 3.28 (m, 2H); 3.71 (t, 1H); 4.53 (s, 2H); 7.51 (m, 3H); 7.84 (m, 3H); 8.10 (br, 3H); 8.54 (t, 1H).

### Example 8

### N-((S)-5-Amino-5-(carbamoyl)pentyl)-3-(aminooxymethyl)benzamide

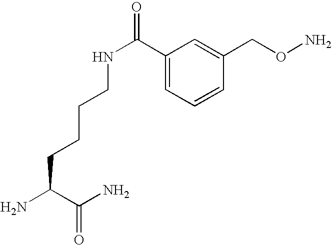

Step 1:

Methyl 3-(((tert-butoxycarbonyl)aminoxy)methyl)benzoate

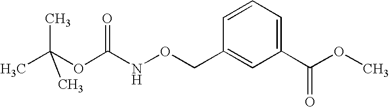

Methyl 3-bromomethylbenzoate (10.0 g, 43.7 mmol) was dissolved in acetnitrile (50 ml). tert-Butyl N-hydroxycarbamate (8.72 g, 65.5 mmol) and 1,8-diazabicycloundec-7-ene (DBU, 9.79 ml, 65.48 mmol) were added successively. The reaction mixture was heated to 50° C. for 16 h. It was cooled to room temperature and diluted with water (200 ml) and concentrated hydrochloric acid (25 ml). It was extracted with ethyl acetate (3×200 ml). The combined organic layers were washed with brine (200 ml) and dried over sodium sulphate. The solvent was removed in vacuo to give 14.15 g of methyl 3-(((tert-butoxycarbonyl)aminoxy)methyl)benzoate, which was used without further purification.

¹H-NMR (CDCl₃): δ 1.48 (s, 9H); 3.91 (s, 3H); 4.90 (s, 2H); 7.45 (m, 2H); 7.60 (d, 1H); 8.00 (d, 1H); 8.05 (s, 1H).

MS: m/z=183.

Step 2:

3-(((tert-Butoxycarbonyl)aminoxy)methyl)benzoic acid

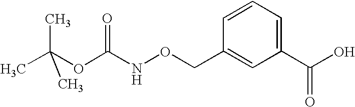

Crude methyl 3-(((tert-butoxycarbonyl)aminoxy)methyl)benzoate (12.28 g, 43.6 mmol) was dissolved in dioxan (50 ml). A solution of lithium hydroxide (1.26 g, 52.4 mmol) in water (50 ml) was added. Dioxan and water were added until a clear solution was obtained. The reaction mixture was stirred at room temperature for 16 h. An 1 N aqueous solution of sodium hydroxide (200 ml) was added. The aqueous solution was washed with tert-butyl methyl ether (2×200 ml). The aqueous phase was acidified by addition of a 10% aqueous solution of sodium hydrogensulphate. It was extracted with ethyl acetate (2×200 ml). The combined ethyl acetate layers were dried over sodium sulphate. The solvent was removed in vacuo to give 12.28 g of crude 3-(((tert-butoxycarbonyl)aminoxy)methyl)benzoic acid, which was used in the next step without further purification.

¹H-NMR (CDCl₃): δ 1.49 (s, 9H); 4.92 (s, 2H); 7.50 (t, 1H); 7.65 (br, 1H); 7.65 (d, 1H); 8.07 (d, 1H); 8.12 (s, 1H).

MS: m/z=169.

Step 3:

2,5-Dioxopyrrolidine-1-yl 3-(((tert-butoxycarbonyl)aminoxy)methyl)benzoate

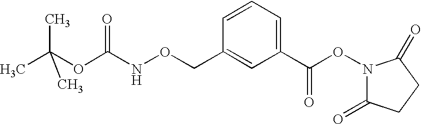

2-Succinimido-1,1,3,3-tetramethyluronium tetrafluoroborate (TSTU, 16.71 g, 55.13 mmol) was added to a solution of crude 3-(((tert-butoxycarbonyl)aminoxy)methyl)benzoic acid (12.28 g, 45.94 mmol) and triethlyamine (7.68 ml, 55.13 mmol) in N,N-dimethylformamide (75 ml). The reaction mixture was stirred at room temperature for 16 h. It was diluted with ethyl acetate (300 ml) and washed with water (3×150 ml). The organic layer was washed with a saturated aqueous solution of sodium hydrogencarbonate (200 ml) and dried over sodium sulphate. The solvent was removed in vacuo to give 11.04 g of crude 2,5-dioxopyrrolidine-1-yl 3-(((tert-butoxycarbonyl)aminoxy)methyl)benzoate, which was used without further purification.

¹H-NMR (CDCl₃): δ 1.47 (s, 9H); 2.91 (s, 4H); 4.92 (s, 2H); 7.28 (s, 1H); 7.53 (t, 1H); 7.75 (d, 1H); 8.10 (d, 1H); 8.15 (s, 1H).

MS: m/z=265

Step 4:

A solution of crude 2,5-dioxopyrrolidine-1-yl 3-(((tert—butoxycarbonyl)aminoxy)methyl)benzoate (11.45 g, 31.41 mmol) in N,N-dimethylformamide (100 ml) was added to a solution of ((S)-5-amino-1-(carbamoyl)pentyl)carbamic acid tert-butyl ester (7.71 g, 31.41 mmol) in N,N-dimethylformamide (50 ml). Ethyldiisopropylamine (16.13 ml, 94.23 mmol) was added. The reaction mixture was stirred at room temperature for 16 h. The solvent was removed in vacuo at 70° C. The residue was dissolved in dichloromethane (50 ml). Trifluoroacetic acid (50 ml) was added. The mixture was stirred for 1 h at room temperature. The solvent was removed in vacuo. The residue was dissolved in dichloromethane (100 ml). A 10% aqueous solution of sodium hydrogensulphate (50 ml) water (200 ml) were added successively. The aqueous phase was concentrated in vacuo to approximately 60 ml. This solution was divided into four parts. Each of them were subjected to a HPLC-chromatography on a C18 reversed phase column, using gradients of 0-20, 0-11, 0-9 or 0-2% respectively acetonitril in water in a buffer of 0.1% trifluoroacetic acid to give together 4.38 g of N-((S)-5-amino-5-(carbamoyl)pentyl)-3-(aminooxymethyl)benzamide.

HPLC: Rₜ=3.24 min (method O₂-b1-2).

¹H-NMR (DMSO-d₆): δ 1.35 (m, 2H); 1.55 (m, 2H); 1.75 (m, 2H); 3.25 (m, 2H); 3.72 (m, 1H); 5.05 (s, 2H); 7.54 (m, 3H); 7.88 (m, 3H); 8.12 (br, 3H); 8.55 (t, 1H).

MS: m/z=295

### Example 9

### (S)-2-(hGH-Leu-amino)-3-(4-((1-(10-(4-(2-((20 kDa mPEGyl)carbamoyloxy)-1-(((20 kDa mPEGyl)carbamoyloxy)methyl)ethoxy)butanoylamino)decyl)1,2,3-triazol-4-yl)methoxy)phenyl)propionamide

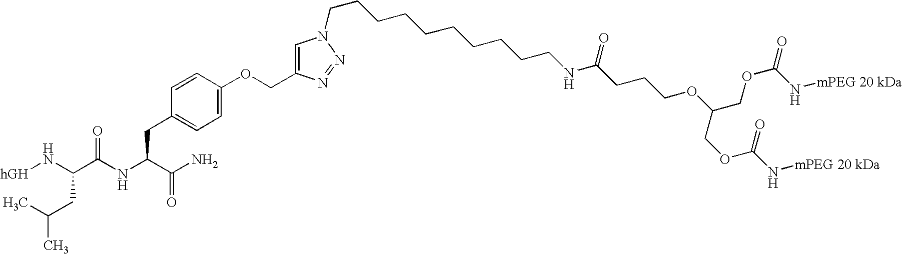

Step 1:

10-Azidodecanol

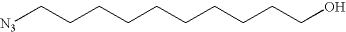

Sodium azide (4.39 g, 67 mmol) was added to a solution of commercially available 10-bromodecanol (4.0 g, 17 mmol) in N,N-dimethylformamide (12 ml). The reaction mixture was stirred at 60° C. for 16 h. It was cooled to room temperature and diluted with water (100 ml) and a saturated aqueous solution of sodium hydrogencarbonate (50 ml). It was extracted with ethyl acetate (3×50 ml). The combined organic layers were dried over magnesium sulphate. The solvent was removed in vacuo. The crude product was purified by flash chromatography on silica (100 g), using a mixture of ethyl acetate/heptane (1:2) as eluent to give 3.25 g of 10-azidodecanol.

MS: 172 [M-N₂]⁺, 222 [M+Na]⁺

¹H-NMR (CDCl₃): δ1.55 (m, 12H); 1.47 (s, 1H); 1.60 (m, 4H); 3.25 (t, 2H); 3.65 (t, 2H).

Step 2:

10-Azidodecyl 4-methylbenzenesulfonic ester

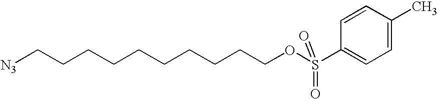

At 0° C., toluenesulfonic chloride (3.27 g, 17.1 mmol) was added to a solution of 10-azidodecanol (3.25 g, 16.3 mmol) and triethylamine (6.83 ml, 49.0 mmol) in dichloromethane (70 ml). The reaction mixture was stirred for 4 days, while the temperature rose slowly to room temperature. It was diluted with ethyl acetate (300 ml) and washed with a 10% aqueous solution of sodium hydrogensulphate. The aqueous phase was extracted with ethyl acetate (100 ml). The combined organic layers were washed with a saturated aqueous solution of sodium hydrogencarbonate (200 ml) and dried over magnesium sulphate. The solvent was removed in vacuo. The crude product was purified by flash chromatography on silica (100 g), using ethyl acetate/heptane (1:2) as eluent, to give 1.85 g of 10-azidodecyl 4-methylbenzenesulfonic ester.

MS: 376 [M+Na]⁺, 326 [M-N₂]⁺.

¹H-NMR (CDCl₃): δ1.15-1.45 (m, 12H); 1.60 (m, 4H); 2.45 (s, 3H); 3.25 (t, 2H); 4.00 (t, 2H); 7.35 (d, 2H); 7.80 (d, 2H).

Step 3

2-(10-Azidodecyl)isoindol-1,3-dione

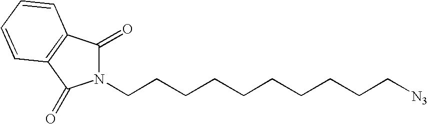

A mixture of 10-azidodecyl 4-methylbenzenesulfonic ester (1.85 g, 5.23 mmol) and commercially available potassium phthalimide (1.45 g, 7.84 mmol) in N,N-dimethylformamide (12 ml) was heated to 10° C. for 4 h. The reaction mixture was cooled to room temperature. It was diluted with ethyl acetate (200 ml) and a 10% aqueous solution of sodium hydrogensulphate (200 ml). The phases were separated. The aqueous phase was extracted with ethyl acetate (100 ml). The combined organic layers were washed with a saturated aqueous solution of sodium hydrogencarbonate (200 ml) and dried over magnesium sulphate. The solvent was removed in vacuo. The crude product was purified by flash chromatography on silica (70 g), using ethyl acetate/heptane (1:2) as eluent, to give 1.33 g of 2-(10-azidodecyl)isoindol-1,3-dione.

MS: 301 [M-N₂]⁺, 351 [M+Na]⁺.

¹H-NMR (CDCl₃): δ 1.20-1.45 (m, 12H); 1.50-1.75 (m, 4H); 3.25 (t, 2H); 3.70 (t, 2H); 7.70 (m, 2H); 7.85 (m, 2H).

Step 4

10-Azidodecylamine

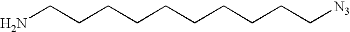

A solution of 2-(10-azidodecyl)isoindol-1,3-dione (1.33 g, 4.05 mmol) and hydrazine hydrate (0.19 ml, 4.85 mmol) in ethanol (30 ml) was heated to 85° C. for 3.25 h. It was cooled to room temperature. The solvent was removed in vacuo. The crude product was purified by flash chromatography on silica (30 g), using a mixture of dichloromethane/methanol/25% aqueous ammonia (100:20:2) as eluent, to give a material, which was dissolved in ethanol. The solvent was removed in vacuo to give 440 mg of 10-azidodecylamine.

MS: 199 [M+H]⁺.

¹H-NMR (CDCl₃): δ 1.25-1.40 (m, 12H); 1.45 (m, 2H); 1.60 (m, 2H); 2.00 (br, 2H); 2.70 (t, 2H); 3.25 (t, 2H).

Step 5

N-(10-Azidodecyl)-4-(2-((20 kDa mPEGyl)carbamoyloxy)-1-(((20 k Da mPEGyl)carbamoyloxy)methyl)ethoxy)butanoic amide

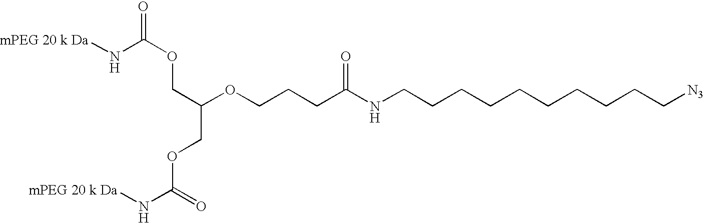

2,5-Dioxopyrrolidin-1-yl 4-(2-((20 kDa mPEGyl)carbamoyloxy)-1-(((20 k Da mPE-Gyl)carbamoyloxy)methyl)ethoxy)butanoic ester (Nektar 2Z3Y0T01, batch PT-09E-02, 1 g, 0.025 mmol) was dissolved in dichloromethane (50 ml). A solution of 10-azidodecylamine (50 mg, 0.25 mmol) in dichloromethane (2 ml) and triethylamine (0.02 m. 0.12 mmol) were added successively. The reaction mixture was stirred at room temperature for 16 h. Ether (800 ml) was added. The formed precipitation was isolated by filtration and was dried in vacuo. It was dissolved in dichloromethane (50 ml). Amberlyst 15 (1.0 g) was added. The mixture was stirred for 5 min at room temperature. The solid was removed by filtration. Ether (800 ml) was added. The formed precipitation was isolated by filtration and dried in vacuo to give N-(10-azidodecyl)-4-(2-((20 kDa mPEGyl)carbamoyloxy)-1-(((20 k Da mPEGyl)carbamoyloxy)methyl)ethoxy)butanoic amide.

Step 6:

(S)-3-(4-(propargyloxy)phenyl)-2-((S)-2-hGHylleucinylamino)propionamide

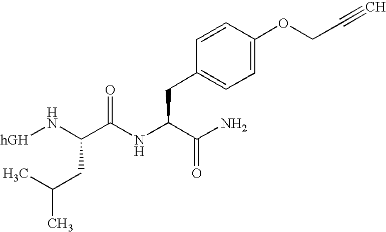

At room temperature a aqueous solution of CPY (200 U/ml, 1 U, 0.005 ml) was added to a solution of hGH-Leu-Ala (15 mg, 672 nmol) and the trifluoroacetate salt of (2S)-2-Amino-3-(4-(prop-2-ynyloxy)phenyl)propionamide (78 mg, 0.23 mmol) in a buffer (1.35 ml) consisting of 0.25 M HEPES and 5 mM EDTA, which was adjusted with an aqueous 1 N sodium hydroxide solution to pH 7.97. After 2.75 h, 0.013 ml of a freshly prepared stock solution of phenylmethanesulfonyl fluoride, prepared by dissolving phenylmethanesulfonyl fluoride (174 mg, 1.0 mmol) in isopropanol (10 ml), were added. The reaction mixture was diluted with a buffer (10 ml) of 50 mM 2-amino-2-(hydroxymethyl)1,3-propanediol, which was adjusted with hydrochloric acid to pH 8.5. The buffer was changed by ultracentrifugation (RCF=3600) using a filter cut-off of 10 kDa to a 5 mM buffer (5 ml) of 2-amino-2-(hydroxymethyl)1,3-propanediol, which was adjusted with hydrochloric acid to pH 8.5. The mixture was filtered through a 450 nm-filter.

MALDI-MS (CHCA): 11231 (M²⁺).

This material was purified by ion-exchange chromatography on a MonoQ column 10/100 GL (Amersham), using a gradient of 0-100% over 60 column volumes of a buffer consisting of 2.0 M sodium chloride and 50 mM TRIS, which was adjusted to pH 8.5 with 1 N hydrochloric acid in a buffer of 50 mM TRIS-buffer, which was adjusted with 1 N hydrochloric acid to pH 8.5, at a flowrate of 0.5 ml/min to give (S)-3-(4-(propargyloxy)phenyl)-2-((S)-2-hGHylleucinylamino)propionamide.

MALDI-MS (CHCA): 11230 (M²⁺).

The buffer was changed to a 50 mM ammonium hydrogencarbonate-buffer (2.5 ml) by ultracentrifugation (RCF=3600) using a filter cut-off of 10 kDa. The material was lyophilized.

Step 7

The protein isolated in step 6 (2.77 mg, 123 nmol) was partly dissolved in a buffer, consisting of 2% 2,6-lutidine in water (0.123 ml). A solution of N-(10-azidodecyl)-4-(2-((20 kDa mP EGyl)carbamoyloxy)-1-(((20 k Da mP EGyl)carbamoyloxy)methyl)ethoxy)butanoic amide (49 mg, 1230 nmol) was dissolved in a buffer, consisting of 2% 2,6-lutidine in water (0.320 ml) was added to the solution of the protein. A solution of copper(II) sulphate (60 mg) in water (6 ml). 1 ml of this copper-solution was added to 1 ml of a solution prepared from ascorbic acid (220 mg) in water (6 ml) and lutidine (0.150 ml), to give a solution of a copper(I)-salt, which was left for 5 min at room temperature. 0.062 ml of this solution of the copper(I)-salt was added to the mixture of the protein and the PEG-reagent. The reaction mixture was shaken gently for 4.5 h. It was diluted with water (2.8 ml) and a buffer (2.77 ml), consisting of 10 mM TRIS-buffer, which was adjusted with 1 N hydrochloric acid to pH 8.0. It was filtered through a 450 nm filter. It was purified by ion-exchange-chromatography on a MonoQ 10/100 GL (Amersham) column, using a gradient of 0-100% over 20 column volumes of a buffer consisting of 0.2 M sodium chloride and 10 mM TRIS, which was adjusted to pH 8.0 with 1 N hydrochloric acid in a buffer of 10 mM TRIS-buffer, which was adjusted with 1 N hydrochloric acid to pH 8.0, at a flowrate of 2 ml/min. The SDS-gel showed a band at a molecular weight of approximately 116 kDa compared to a Marker 12 (Invitrogen), which stained both with the silver Quest staining procedure (Invitrogen) and a PEG-sensitive staining method (Kurfurst, M. M. Analytical Biochemsitry 1992, 200, 244-248), which all is within the expectation for (S)-2-(hGH-Leu-amino)-3-(4-((1-(10-(4-(2-((20 kDa mPEGyl)carbamoyloxy)-1-(((20 kDa mP EGyl)carbamoyloxy)methyl)ethoxy)butanoylamino)decyl) 1,2,3-triazol-4-yl)methoxy)phenyl)propionamide.

### Example 10

### (S)-2-((hGHylleucinyl)amino)-6-(3-((4-((4-(bis(20 kDa mPEGylcarbamoyloxymethyl)methoxy)butyrylamino)methyl)triazol-1-yl)methyl)benzoylamino)hexanoic amide

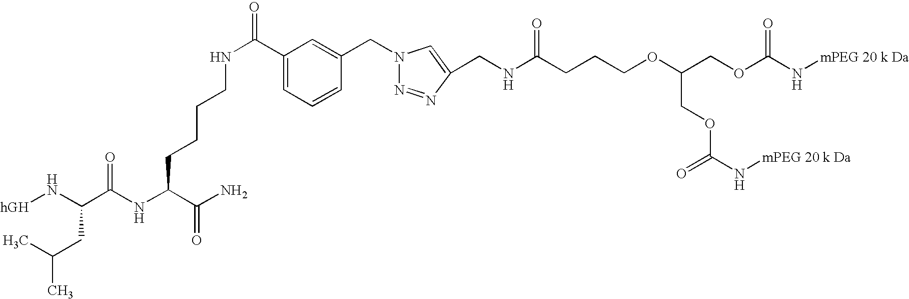

Step 1:

4-(bis(20 kDa mPEGylcarbamoyloxymethyl)methoxy)-N-prop-2-ynylbutyric amide

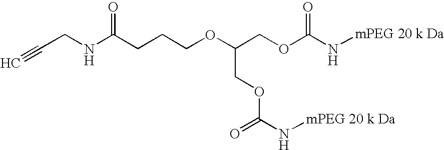

4-(bis(20 kDa mPEGylcarbamoyloxymethyl)methoxy)butyric acid (purchased from Nektar, catalog number 2Z3Y0T01, 2 g, 0.05 mmol) was dissolved in dichloromethane (25 ml). Ethyldiisopropylamine (0.042 ml, 0.248 mmol) and propargylamine (0.014 ml, 0.198 mmol) were added successively. The reaction mixture was stirred for 3 days. Diethyl ether was added until a precipitation occurred. The mixture was cooled to 0° C. and was filtered through a P1-glass-filter. The precipitation was collected and dissolved in dichloromethane (18 ml) and ethanol (2 ml). Amberlyst 15 (1.0 g) was washed with ethanol and added. The mixture was stirred gently for 30 min and was filtered. The solvent was removed in vacuo. Diethylether (50 ml) was added. The precipitation was isolated by filtration and dried 2 days in vacuo to give 1.36 g of 4-(bis(20 kDa mPEGylcarbamoyloxymethyl)methoxy)-N-prop-2-ynylbutyric amide.

Step 2:

(S)-2-((hGHylleucinyl)amino)-6-(3-(azidomethyl)benzoylamino)hexanoic amide

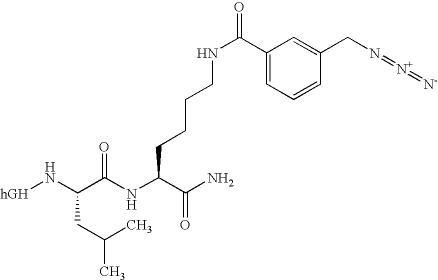

hGH-Leu-Ala (15 mg, 672 nmol) was dissolved in water (0.450 ml) and diisopropylethylamine (0.007 ml). A solution of (S)-2-amino-6-(3-(azidomethyl)benzoylamino)hexanoic amide (98 mg, 0.235 mmol) in 0.120 ml of a buffer of 0.25 M HEPES and 5 mM EDTA, which had been adjusted to pH 8 with a 1 N solution of sodium hydroxide, was added. The solution was filtered and diluted with a buffer of 0.25 M HEPES and 5 mM EDTA, which had been adjusted to pH 8 with a 1 N solution of sodium hydroxide, and a 1 N solution of sodium hydroxide in order to obtain 1.344 ml of a solution with a pH of 7.8. A solution of CPY (200 U/ml, 0.075 ml, 15 U) was added. The reaction mixture was gently shaken at 30° C. for 21 h. A freshly prepared 100 mM solution of phenylmethanesulfonyl fluoride (0.0135 ml) in isopropanol was added. The reaction mixture was kept for 1 day at room temperature. It was chromatographed on a HiPrep 26/10 desalting column, using a 50 mM ammonium bicarbonate buffer in water as eluent. A freshly prepared 100 mM solution of phenylmethanesulfonyl fluoride (0.0045 ml per 0.5 ml fraction-volume) in isopropanol was added to the fractions immediately, containing the protein. These fractions were combined and were subjected to ultracentrifugation, using an Amicon Ultra-15 vial with a cut off of 10 kDa filter. They were diluted with a 50 mM solution of ammonium bicarbonate and lyophilized.

Step 3:

A solution of copper(II) sulfate pentahydrdate (41 mg, 0.16 mmol) in water (9.17 ml) was prepared. A solution of ascorbic acid (145 mg, 0.82 mmol) in water (8.94 ml) and 2,6-lutidine (0.229 ml) was prepared. Of both of the latter solution 3.51 ml were taken and were mixed. They were left at room temperature for 5 min to form a copper(I)-salt solution, which was used directly after the 5 min had passed.

A solution of (S)-2-((hGHylleucinyl)amino)-6-(3-(azidomethyl)benzoylamino)hexanoic amide (7.08 mg, 314 nmol) in a mixture of water (0.862 ml) and 2,6-lutidine (0.18 ml) was added to a solution of 4-(bis(20 kDa mPEGylcarbamoyloxymethyl)methoxy)-N-prop-2-ynylbutyric amide (127 mg, 0.003 mmol) in water (0.700 ml). 0.351 ml of the copper(I)-salt solution was added. The reaction mixture was gently shaken for 20 h. The reaction mixture was filtered and was diluted to 2 ml with a 10 mM buffer of TRIS, which had been adjusted with 1 N hydrochloric acid to pH 8.0. It was subjected to a gel chromatography, using a HiLoad 26/60 Superdex 200 column in a 10 mM Tris buffer, which had been adjusted to pH 8 with 1 N hydrochloric acid. The fractions containing the desired protein were combined and concentrated by ultracentrifugation using an Amicon Ultra-15 vial with a cut off of 10 kDa. It was diluted with a 50 mM Tris-buffer (20 ml), which had been adjusted to pH 8.5 with 1 N hydrochloric acid. It was purified by ion-exchange-chromatography on a MonoQ 10/100 GL column, using a 50 mM Tris buffer, which had been adjusted to pH 8.5 with 1 N hydrochloric acid as buffer A and a 50 mM Tris/2 M sodium chloride buffer, which had been adjusted to pH 8.5 with 1 N hydrochloric acid as buffer B. The fractions containing the desired protein were combined and concentrated by ultracentrifugation using an Amicon Ultra-1±5 vial with a cut off of 10 kDa. The buffer was changed to a 50 mM ammonium bicarbonate buffer by ultracentrifugation using an Amicon Ultra-1±5 vial with a cut off of 10 kDa and lyophilized. The SDS-gel showed a compound, which had the expected properties of the title compound.

### Example 11

### (S)-2-(hGHylleucinylamino)-6-(3-((4-((4-(30 kDa mPEGyloxy)butyrylamino)methyl)1,2,3-triazol-1-yl)methyl)benzoylamino)hexanoic amide

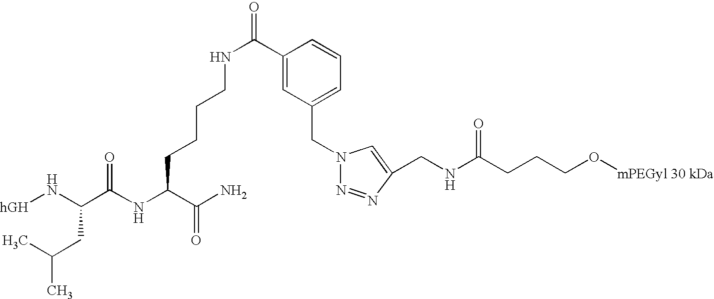

Step 1:

4-(30 kDa mPEGyl)-N-(prop-2-ynyl)butanoic amide

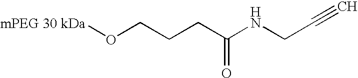

2,5-Dioxoprrolidin-1-yl 4-(30 kDa mPEGyl)butanoic ester (purchased at Nektar, 2.5 g, 0.083 mmol) was dissolved in dichloromethane (25 ml). Ethyldiisopropylamine (0.071 ml, 0.413 mmol) and propargylamine (0.023 ml, 0.33 mmol) were added successively. The reaction mixture was stirred at room temperature over night. Diethyl ether was added until a precipitation was formed. The mixture was cooled to 0° C. and the precipitation was isolated by filtration through a glass-filter P1. The isolated material was dissolved in a 10% solution of ethanol in dichloromethane (15 ml). Amberlyst 15 (2.0 g), which had been washed with a 10% solution of ethanol (20 ml) prior its use, was added. The mixture was stirred slowly for 30 min. The Amberlyst-material was removed by filtration and was washed with dichloromethane (20 ml). The solution was concentrated in vacuo. Ether was added, until a precipitation occurred. The mixture was cooled to 0° C. The precipitation was isolated by filtration through a glass-filter P1 and dried in vacuo to give 2.04 g of 4-(30 kDa mPEGyl)-N-(prop-2-ynyl)butanoic amide.

Step 2

A solution of (S)-2-((hGHylleucinyl)amino)-6-(3-(azidomethyl)benzoylamino)hexanoic amide (11.0 mg, 488 nmol) was dissolved in a mixture of water (1.328 ml) and 2,6-lutidine (0.028 ml). This solution was filtered into a solution of 4-(30 kDa mPEGyl)-N-(prop-2-ynyl)butanoic amide (147 mg, 4860 nmol) in water (1.00 ml). A copper(I)-salt solution was prepared by addition of a solution of copper(II) sulphate pentahydrate (24.3 mg, 0.097 mmol) in water (5.44 ml) to a solution of ascorbic acid (85.0 mg, 0.488 mmol) in a mixture of water (5.30 ml) and 2,6-lutidine (0.135 ml). This copper(I)-salt solution was left for 5 min at room temperature. A part of this copper(I)-salt solution (0.544 ml) was added to the solution containing the protein. The reaction mixture was gently shaken for 22 h. The solution was filtered. The filter was washed with a buffer consisting of 25 mM Tris in water, which was adjusted to pH 8.5 with 1 N hydrochloric acid. The solution was run on a column, using a HiPrep 26/10 desalting column with a flow of 20 ml/min and a buffer consisting of 25 mM Tris in water, which was adjusted to pH 8.5 with 1 N hydrochloric acid. The fractions, containing protein, were collected and combined. The protein was purified on by ion-exchange chromatography using a MonoQ 10/100 GL column, a buffer consisting of 25 mM Tris in water, which was adjusted to pH 8.5 with 1 N hydrochloric acid as buffer A and a buffer consisting of 0.2 M sodium chloride and 25 mM Tris in water, which was adjusted to pH 8.5 with 1 N hydrochloric acid as buffer B, applying a gradient of 0-100% buffer B over 100 column volumes with a flow of 0.50 ml/min. The fractions containing the desired protein were collected, combined and concentrated via ultracentrifugation using Amicon Ultra centrifugation vials with a cut-off of 10 kDa. After concentration, the buffer was changed to a 50 mM ammonium hydrogencarbonate buffer in the same ultracentrifugation vials. The material was lyophilized to give 4.6 mg of the title compound. SDS-gels are in accordance with the expectation for the title compound. The characterization of the compounds were done by SDS-gel PAGE, using silver staining and a specific PEG staining as described by Kurfurst (Analytical Biochemistry 1992, 200, 244-248.).

### Example 12

### (S)-6-(3-((4-((4-(N-(3-(omega-(2,3-bis(20 kDa mPEgyloxy)propoxy)₂₋₅ kDa PEGyloxy)propyl)carbamoyl)butyrylamino)methyl)triazol-1-yl)methyl)benzoylamino)-2-((hGHyl)leucinylamino)hexanoic amide

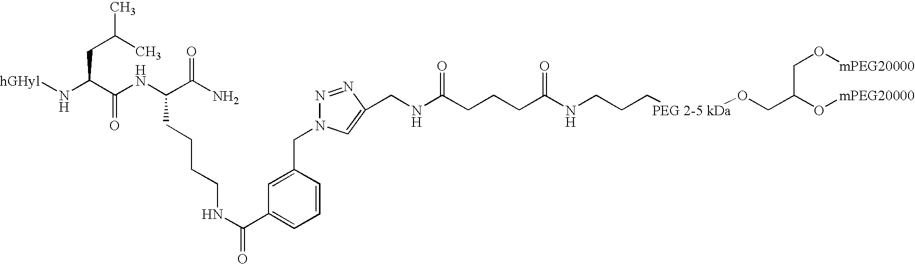

Step 1:

4-(N-(3-(omega-(2,3-Bis(20 kDa mPEGyloxy)porpoxy)₂₋₅ kDa PEGyloxy)propyl)carbamoyl)-N-(prop-2-ynyl)butyric amide

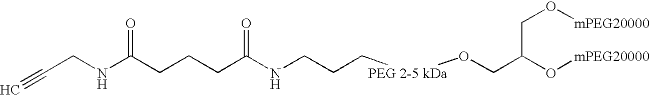

2,3-Bis(20 kDa PEGyloxy)-1-({3-[(1,5-dioxo-5-succinimidyloxypentyl)amino]propoxy} 2-5 kDa PEGyloxy)propane (purchased from NOF, order number: Sunbright GL3-400GS2, 1.00 g, 0.023 mmol) was dissolved in dichloromethane (10 ml). Ethyldiisopropylamine (0.019 ml, 0.113 mmol) and propargylamine (0.006 ml, 0.091 mmol) were added successively. The reaction mixture was stirred for 16 h at room temperature. Diethylether was added until a precipitation was obtained. The mixture was kept at 0° C. for 1 h. The precipitation was isolated by filtration through a filter paper P1.

Amberlyst 15 ion-exchange material (1.0 g) was suspended in a mixture of dichloromethane (10 ml) and ethanol (1 ml). The mixture was stirred gently for 30 min. The amberlyst was isolated by filtration.

The precipitation of the PEG-reagent was dissolved in a mixture of dichloromethane (10 ml) and ethanol (1 ml). The amberlyst material was added. The mixture was stirred gently for 30 min at room temperature. The amberlyst was removed by filtration and washing with dichloromethane. The combined solutions were concentrated in vacuo to approx. 2 ml. Diethylether was added, until a precipitation was obtained. The mixture was kept at 0° C. for 1 h. The precipitation was isolated by filtration through a filter paper P1 and dried in vacuo to give 0.83 g of 4-(N-(3-(omega-(2,3-bis(20 kDa mPEGyloxy)porpoxy)₂₋₅ kDa PEGyloxy)propyl)carbamoyl)-N-(prop-2-ynyl)butyric amide.

Step 2:

A solution of (S)-2-((hGHylleucinyl)amino)-6-(3-(azidomethyl)benzoylamino)hexanoic amide (8.23 mg, 365 nmol) was dissolved in a mixture of water (0.996 ml) and 2,6-lutidine (0.021 ml). This solution was added to a solution of 4-(N-(3-(omega-(2,3-bis(20 kDa mPEGyloxy)porpoxy)₂₋₅ kDa PEGyloxy)propyl)carbamoyl)-N(prop-2-ynyl)butyric amide (161 mg, 3650 nmol) in water (0.75 ml). A copper(I) salt solution was prepared by mixing of a solution of copper(II) sulphate pentahydrate (18.23 mg, 0.073 mmol) in water (4.08 ml) with a solution of ascorbic acid (64.52 mg, 0.366 mmol) in a mixture of water (3.98 ml) and 2,6-lutidine (0.10 ml). This solution was shaken for 5 min at room temperature. 0.41 ml of this copper(I) solution was taken and added to the solution containing the protein and the PEG-reagent. The reaction mixture was shaken gently for 16 h at room temperature. It was filtered through a 450 nm filter. A gel-chromatography was performed, using a HiPrep 26/10 desalting column (Amersham) and a buffer of 25 mM TRIS, which had been adjusted to pH 8.5 with 1 N hydrochloric acid, at a flow of 10 ml/min. The fractions containing the desired compound were diluted with a buffer 25 mM TRIS, which had been adjusted to pH 8.5 with 1 N hydrochloric acid (45 ml). An ion-exchange chromatography was performed, using a MonoQ 10/100 GL column (Amersham) at a flow of 0.50 ml/min and a gradient of 0-100% of a buffer of 0.2 M sodium chloride and 25 mM TRIS, which had been adjusted to pH 8.5 with 1 N hydrochloric acid, in a buffer of 25 mM TRIS, which had been adjusted to pH 8.5 with 1 N hydrochloric acid over 25 column volumes. The fractions, containing the desired compound were combined. This solution was divided into two parts. Each of these parts were subjected to a gel-chromatography, using a HiPrep 26/10 desalting column (Amersham) and a solution of 50 mM ammonium hydrogencarbonate at a flow of 10 ml/min. The fractions of both runs, which contained the desired product were combined and lyophilized to give 2.6 mg of the desired compound. The characterization of the compounds were done by SDS-gel PAGE, using silver staining and a specific PEG staining as described by Kurfurst (Analytical Biochemistry 1992, 200, 244-248.).

### Example 13

### (S)-2-((N^{1alpha}-(4-(2-(2-(2-(2-(4-(bis((20 kDa mPEGylaminocarbonyloxy)methyl)methoxy)butyrylamino)ethoxy)ethoxy)ethoxy)ethoxy)butyl)hGHyl)leucylamino)-6-(3-((4-((4-(30 kDa mPegyloxy)butyrylamino)methyl)-1,2,3-triazol-1-yl)methyl)benzoylamino)hexanoic amide

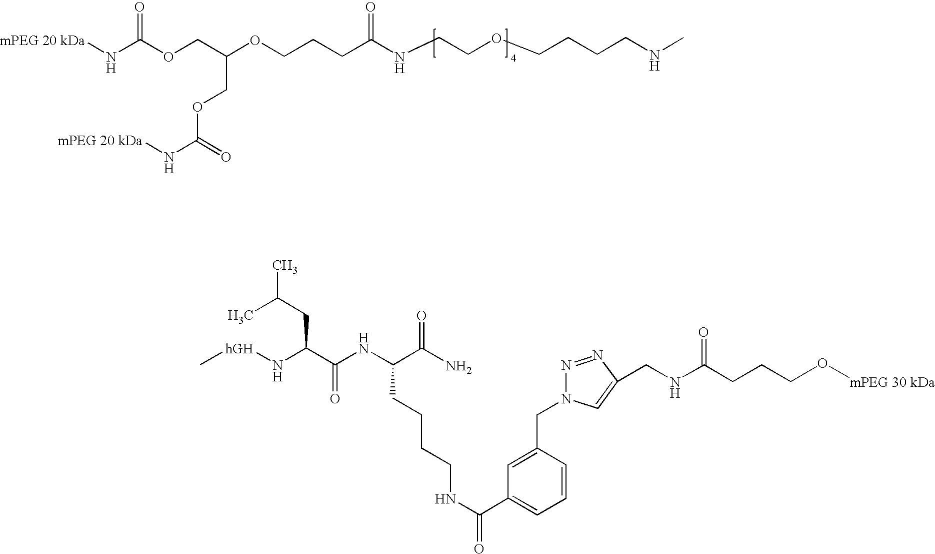

Step 1:

(S)-6-(3-(azidomethyl)benzoylamino)-2-((N^{1alpha}-(4-(2-(2-(2-(2-(4-(bis((20 kDa mPEGylaminocarbonyloxy)methyl)methoxy)butyrylamino)ethoxy)ethoxy)ethoxy)ethoxy)butyl)hGHyl)leucylamino)hexanoic amide

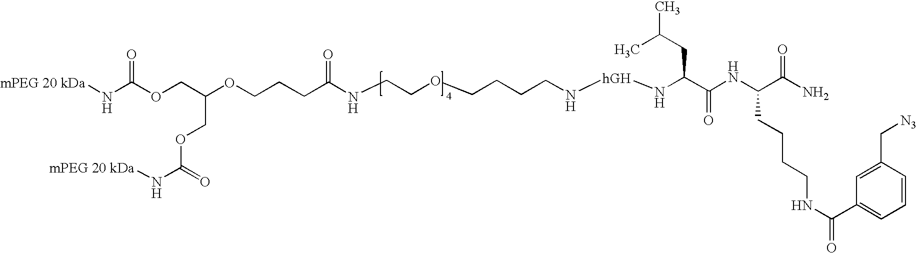

(S)-2-((hGHylleucinyl)amino)-6-(3-(azidomethyl)benzoylamino)hexanoic amide (25.8 mg, 0.001 mmol) was suspended in a 25 mM HEPES-buffer (0.500 ml), which had been adjusted to pH 7. Ethyldiisopropylamine (0.004 ml) was added. A clear solution was obtained. 25 mM HEPES-buffer (2.050 ml), which had been adjusted to pH7, was added. The solution was adjusted to pH 6.98 by addition of 1 N hydrochloric acid (0.040 ml). 4-(2-(2-(2-(2-(4-(bis((20 kDa mPEGylaminocarbonyloxy)methyl)methoxy)butyrylamino)ethoxy)ethoxy)ethoxy)ethoxy)butanal (23.39 mg, 0.0006 mmol) was added. A freshly prepared of sodium cyanoborohydride (0.025 ml, 0.025 mmol) was added over a period of 5 h. The reaction mixture was left gently shaken at room temperature in the dark for 16 h. The reaction mixture was filtered. The buffer was changed to a 25 mM TRIS-buffer pH 8.5 via chromatography on a HiPrep26/10 desalting column. This solution was subjected to a ion-exchange chromatography on a MonoQ10/100 GL column. While the sample was put onto the column the flow was 0.5 ml/min. For elution a gradient was used of 0-75% of a 25 mM TRIS/0.2 M sodium chloride buffer in a 25 mM TRIS-buffer, which both had been adjusted to pH 8.5 over 30 column volumes followed by 75-100% of a 25 mM TRIS/0.2 M sodium chloride buffer in a 25 mM TRIS-buffer, which both had been adjusted to pH 8.5 with a flow of 4.0 ml/min. The fraction containing the desired compound were identified by SDS-gel electrophoresis. They were pooled. The buffer was changed to a 50 mM ammonium hydrogencarbonate buffer by subjecting it to a chromatography on a HiPerpe26/10 desalting column. The material was lyophilized to give 3.8 mg of (S)-6-(3-(azidomethyl)benzoylamino)-2-((N^{1alpha}-(4-(2-(2-(2-(2-(4-(bis((20 kDa mPEGylaminocarbonyloxy)methyl)methoxy)butyrylamino)ethoxy)ethoxy)ethoxy)ethoxy)butyl)hGHyl)leucylamino)hexanoic amide.

Step 2:

A solution of copper(II) sulphate (3.0 mg) in water (0.680 ml) was added to a solution of ascorbic acid (10.7 mg) in a mixture of water (0.66 ml) and 2,6-lutidine (0.015 ml). This solution was left at room temperature for 5 min. 0.068 ml of this solution was added to a solution of (S)-6-(3-(azidomethyl)benzoylamino)-2-((N^{1alpha}-(4-(2-(2-(2-(2-(4-(bis((20 kDa mPEGylaminocarbonyloxy)methyl)methoxy)butyrylamino)ethoxy)ethoxy)ethoxy)ethoxy)butyl)hGHyl)leucylamino)hexanoic amide (3.8 mg, 60 nmol) and 4-(30 kDa mPEGyl)-N-(prop-2-ynyl)butanoic amide (18.1 mg, 600 nmol) in water (1.18 ml) and 2,6-lutidine (0.020 ml). The reaction mixture was shaken gently at room temperature for 22 h. The buffer was changed to a 50 mM ammonium hydrogencarbonate buffer by subjecting it to a chromatography on a HiPrep26/10 desalting column. The buffer was changed again to a 25 mM Tris-buffer, which had been adjusted to pH 8.5, by subjecting it to a chromatography on a HiPrep26/10 desalting column. This solution was subjected to a ion-exchange chromatography on a MonoQ10/100 GL column. While the sample was put onto the column the flow was 0.5 ml/min. For elution a gradient was used of 0-75% of a 25 mM TRIS/0.2 M sodium chloride buffer in a 25 mM TRIS-buffer, which both had been adjusted to pH 8.5 over 30 column volumes followed by 75-100% of a 25 mM TRIS/0.2 M sodium chloride buffer in a 25 mM TRIS-buffer, which both had been adjusted to pH 8.5 with a flow of 4.0 ml/min. The fractions containing the desired compound were identified by SDS-gel electrophoresis. They were pooled. The buffer was changed to a 50 mM ammonium hydrogencarbonate buffer by subjecting it to a chromatography on a HiPrep26/10 desalting column. The solution was lyophilized to give 0.42 mg of (S)-2-((N^{1alpha}-(4-(2-(2-(2-(2-(4-(bis((20 kDa mPEGylaminocarbonyloxy)methyl)methoxy)butyrylamino)ethoxy)-ethoxy)ethoxy)ethoxy)butyl)hGHyl)leucylamino)-6-(3-((4-((4-(30 kDa mPegyloxy)butyrylamino)methyl)-1,2,3-triazol-1-yl)methyl)benzoylamino)hexanoic amide.

### Example 14

### (S)-2-{(hGHylleucinyl)amino}-6-{4-(1-(4-(4-(2-(N-(20 kDa mPEGyl)carbamoyloxy)-1-((N-(20 kDa mPEGyl)carbamoyloxy)methyl)ethoxy)butyrylamino)butoxyimino)ethyl)benzoylamino}hexanoic amide

Step 1:

as in Example 3.

Step 2:

Oximation with N-(4-(aminoxy)butyl)-4-(2-((20 kDa mPEGyl)carbamoyloxy)-1-(((20 kDa mP EGyl)carbamoyloxy)methyl)ethoxy)butanoic amide

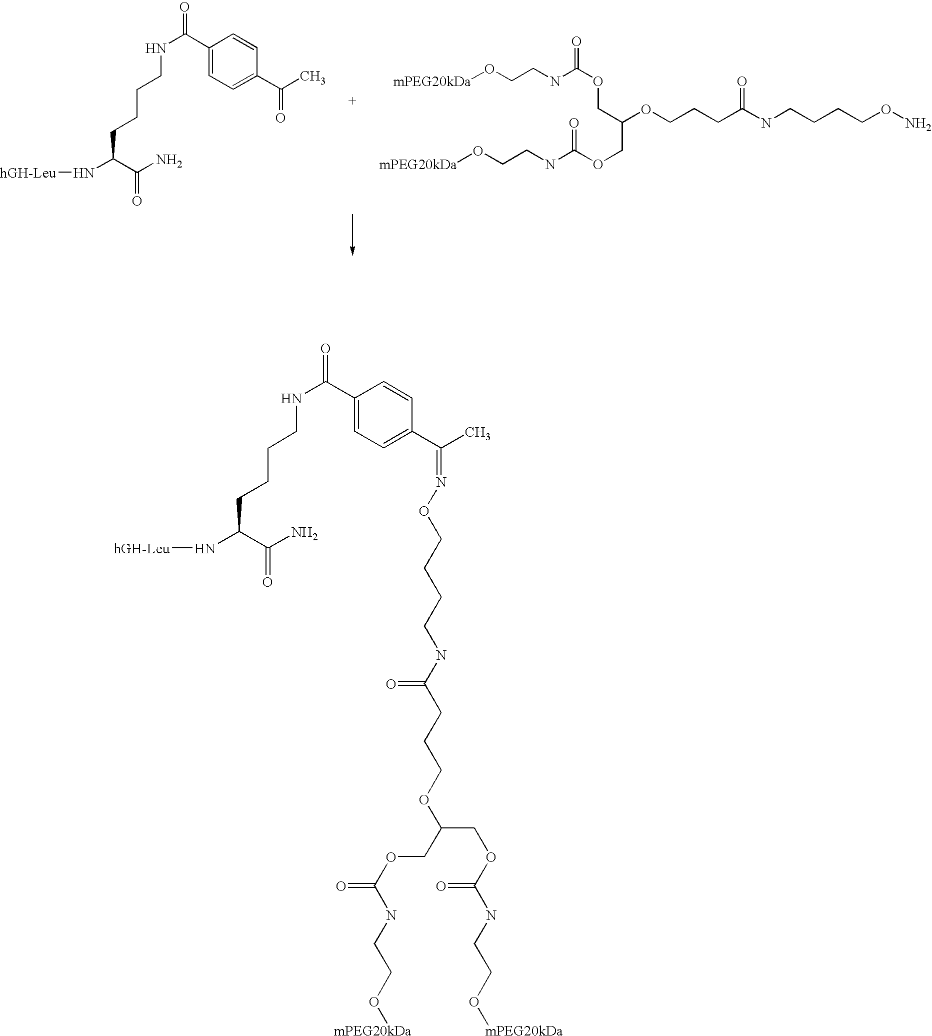

The pooled fractions (3 mg/ml, 6 ml) from the first step were put on ice bath. Ice cold DMF was added (1.32 ml, 15% final concentration). The PEG reagent was added (281 mg, about 10 equivalents in solution in 3-Methylthio-1 propanol 0.14M (1 ml)). The volume was adjusted to 8.8 ml by addition of MES buffer 50 mM pH6. The final pH of the reaction mixture was 6.

Reaction progress was followed by running analyses on Agilent 2100 Bioanalyzer.

The reaction mixture was incubated at 30° C. under nitrogen for 10 days.

Purification was done on an aliquot of the reaction mixture by size exclusion chromatography (Amersham Superdex 200 26/26, eluent: Tris, HCl 50 mM pH8.5, 2.5 ml/min), followed by ion exchange (MonoQ10/100GL, A buffer: Tris 50 mM pH8.5, buffer B: A+0.2M NaCl, 0 to 100% B over 100 column volumes, 0.5 ml/min).

Yield: 2 mg of product was isolated (3.5% from the starting hGH-Leu-Ala protein)

### Example 15

### (S)-2-((hGHylleucinyl)amino)-6-(4-(1-((3-((4-(2-(2-(2-(2-(4-(bis((20 kDa mPegylcarbamoyloxy)methyl)methoxy)butyrylamino)ethoxy)ethoxy)ethoxy)ethoxy)butylidene)aminoxy)propoxy)imino)ethyl)benzoylamino)hexanoic amide

Step 1:

as in example 3.

Step 2:

Oximation with 1,3-diaminoxy propane:

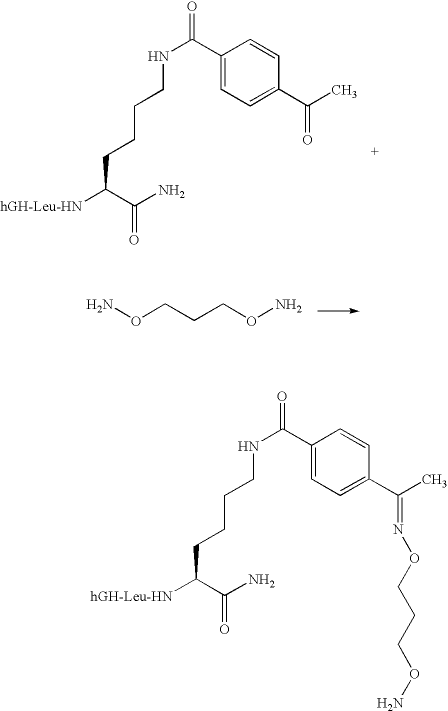

To a solution of the starting ketone in aqueous 0.14M 3-methylthio-1propanol (28 mg, 0.37 mM final concentration) (3 ml) was added 1,3-diaminoxypropane (TFA salt) (68 mg, 300 equivalents, 111 mM final concentration) in solution in aqueous 0.14M 3-methylthio-1 propanol (0.4 ml). The final pH was 4.

The reaction was followed by CE. CE analysis method:

The capillary electrophoresis was performed using a Hewlett Packard 3D CE system equipped with a diode array detector.

The fused silica capillary (Agilent) used had a total length of 64.5, an effective length of 56 cm and an ID of 50 μm. Samples were injected by pressure at 50 mbar for 4 s. Separations were carried out at 30° C., under a voltage of +25 kV, using phosphate buffer 50 mM pH2.5 as electrolyte. The analysis was monitored at 200 nm. Between runs, a basic wash were performed: the capillary was rinsed with water for 2 min, then with sodium hydroxide 0.1 M for 3 min, and water for 2 min, before equilibrating the capillary with the electrolyte.

The reaction ran to completion after 1 h.

The identity of the product was confirmed by MALDI-TOF analysis.

MALDI-TOF analysis method: as in example 3, step 1.

m/z=11301 (product) (M+2H)²⁺

After addition to the reaction mixture of a buffer containing triethanolamine (45 mM) and 3-methylthio-1 propanol (0.14M), the excess of reagent was eliminated by ultra filtration on Millipore Amicon Ultra cut off 10 kD (twice). The remaining protein solution was ultrafiltered again after addition of 3-methylthio-1 propanol (0.14M) (twice).

Finally, the protein solution obtained was run on desalting column (Amersham HiPrep 26/10 Desalting, eluent: Tris 50 mM pH8.5, 10 ml/min). A buffer shift to 3-methylthio-1 propanol (0.14M) was then performed. The final protein concentration was 10 mg/ml.

Step 3:

Oximation with mPEG2-ButyrALD-40K

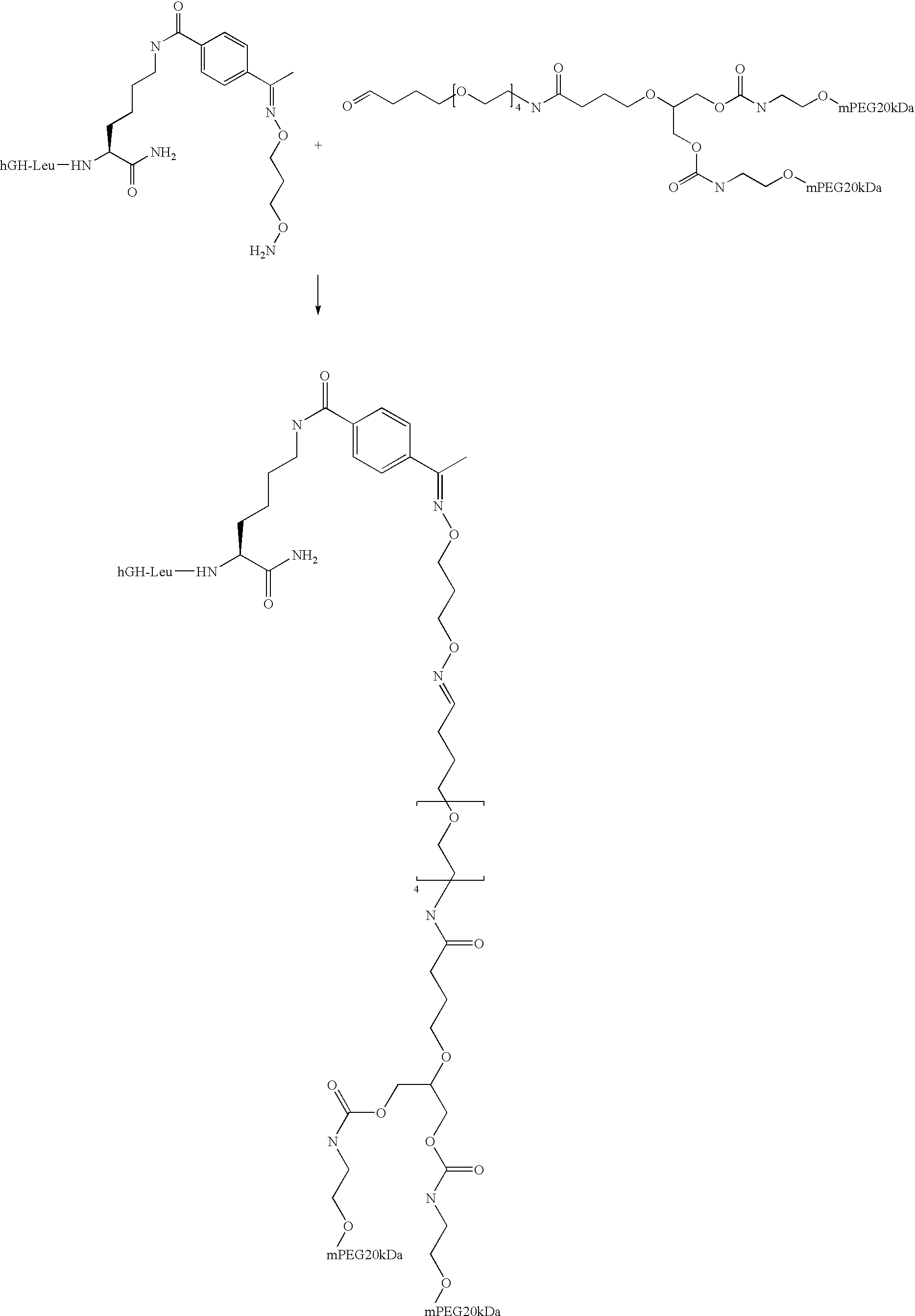

To the protein solution obtained in step 2 (28 mg, 7 mg/ml) was added mPEG2-ButyrALD-40K (Nektar #083Y0T01)(164 mg, 4.1 μmoles, 3 equivalents) in solution in 0.14M 3-methylthio-1 propanol (0.4 ml)

The reaction mixture was incubated at 30° C. for 48 h.

The product was purified on ion exchange (Amersham MonoQ 10/100 GL, A buffer: Tris 50 mM pH8.5, buffer B: A+0.2M NaCl, 0 to 50% B over 20 column volume, 50 to 100% over 3 column volumes, 4 ml/min).

The pooled fractions were lyophilized after buffer shift to ammonium bicarbonate.

Yield: 33 mg of product was isolated (33% from the starting hGH-Leu-Ala protein)

### Example 16

### (S)-6-(4-(1-(3-((3-(omega-(2,3-bis(20 kDa mPEGyloxy)propyl)₂₋₅ kDa PEGyloxy) propylidene)aminoxy)propoxyimino)ethyl)benzoylamino)-2-(((hGHyl)leucinyl)amino)hexanoic amide

Step 1:

as in Example 3.

Step 2:

Oximation with 1,3-diaminoxy propane: as in example 15 except that the reaction was run at pH6.5. The reaction mixture was incubated 18 h at 30° C.

The work up was as described in step 2 of example 15.

Step 3:

Oximation with “Sunbright GL3-400AL2”:

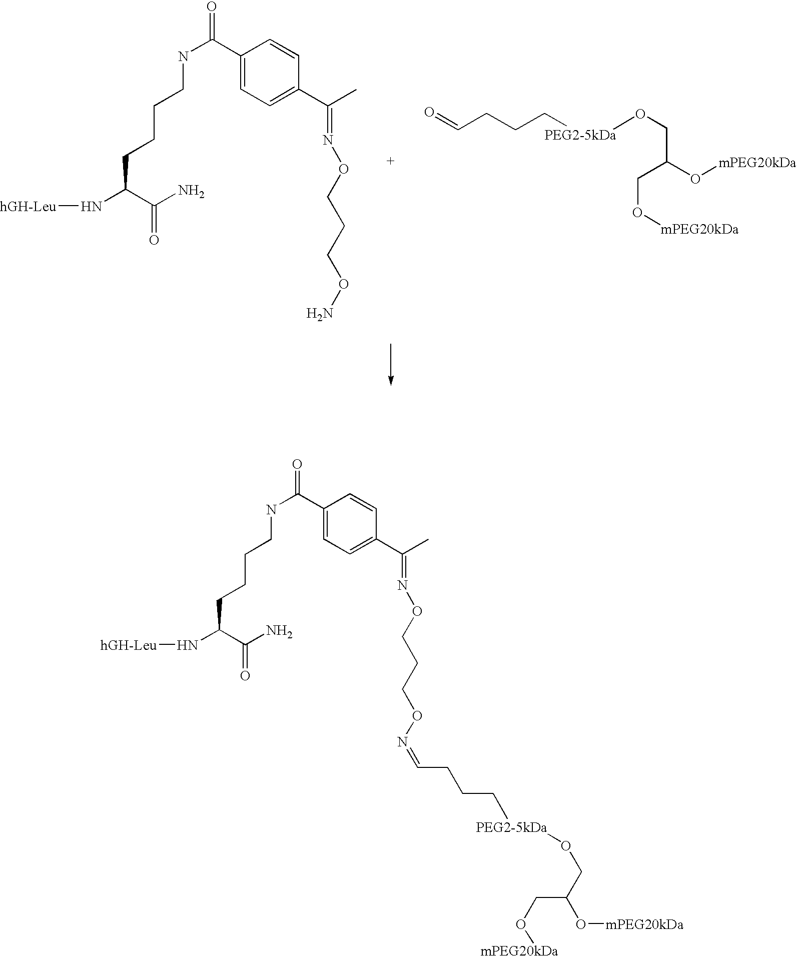

To “Sunbright GL3-400AL2” (NOF product) (130 mg, 3.2 μmoles, about 3 equivalents) in solution in 0.14M 3-methylthio-1 propanol (1 ml) was added the protein solution obtained in step 2 (3 ml, about 10 mg/ml))

The reaction mixture was incubated at 30° C. and the reaction followed by analysis on Agilent 2100 Bioanalyzer.

After 18 h reaction time, the yield was 39% according to the bioanalyzer integration results.

The product was purified on ion exchange (Amersham MonoQ 10/100 GL, A buffer: Tris 50 mM pH8.5, buffer B: A+0.2M NaCl, 0 to 50% B over 20 column volume, 50 to 100% over 3 column volumes, 4 ml/min).

The pooled fractions were lyophilized after buffer shift to ammonium bicarbonate.

Yield: 9 mg of product was isolated (10% from the starting hGH-Leu-Ala protein)

### Example 17

### (S)-6-(3-(((3-(omega-(2,3-bis-(20 kDa m-PEGyloxy)propyl)₂₋₅ kDa PEGyloxy)propylidine)aminoxy)methyl)benzoylamino)-2-((hGHyl)leucinylamino)hexanoic amide

Step 1:

CPY-catalyzed transpeptidation of hGH-Leu-Ala with N-((S)-5-Amino-5-(carbamoyl)pentyl)-3-(aminoxymethyl)benzamide

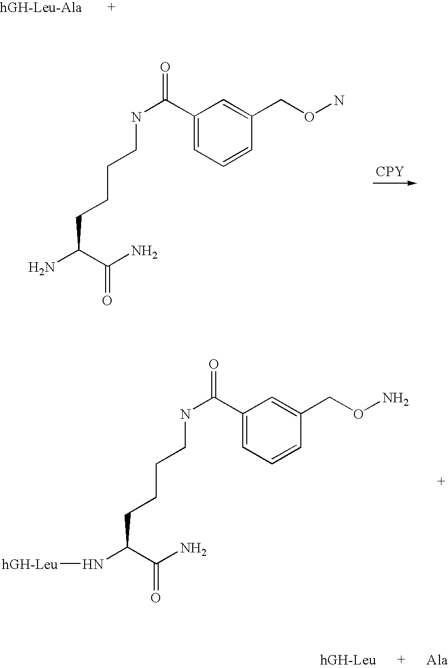

To a solution of hGH-Leu-Ala (15 mg, 0.5 mM final concentration) in H₂O:diisopropylamine (100:1 v/v, 0.6 ml), was added N-((S)-5-Amino-5-(carbamoyl)pentyl)-3-(aminoxymethyl)benzamide

(86.4 mg, 159 mM final concentration) in solution in HEPES buffer 250 mM pH8.5 containing 5 mM EDTA (0.73 ml). The pH was adjusted to 8.2 by addition of sodium hydroxide 10M. The reaction volume was adjusted to 1.32 ml by addition of HEPES buffer 250 mM pH8.5 containing 5 mM EDTA. The reaction was started by addition of the enzyme (Fluka #21943) in solution in water (10 U/ml final concentration) (15 U added). The reaction mixture was incubated at 30° C.

The reaction was followed by MALDI analysis. After 3 h reaction time, only traces of the starting material could be detected.

After addition to the reaction mixture of a buffer containing triethanolamine (45 mM), 3-methylthio-1 propanol (0.14M) and PMSF (2 mM), the excess of reagent was eliminated by ultra filtration on Millipore Amicon Ultra cut off 10 kD (twice). The remaining protein solution was ultrafiltered again after addition of a solution containing 3-methylthio-1 propanol (0.14M) and PMSF (2 mM) (twice).

Finally, the protein solution obtained was run on desalting column (Amersham HiPrep 26/10 Desalting, eluent: Tris 50 mM pH8.5, 10 ml/min). A buffer shift to 3-methylthio-1 propanol (0.14M) was then performed. The final protein concentration was about 10 mg/ml.

This protein solution was used directly in the next step.

Step 2:

Oximation with “Sunbright GL3-400AL2”:

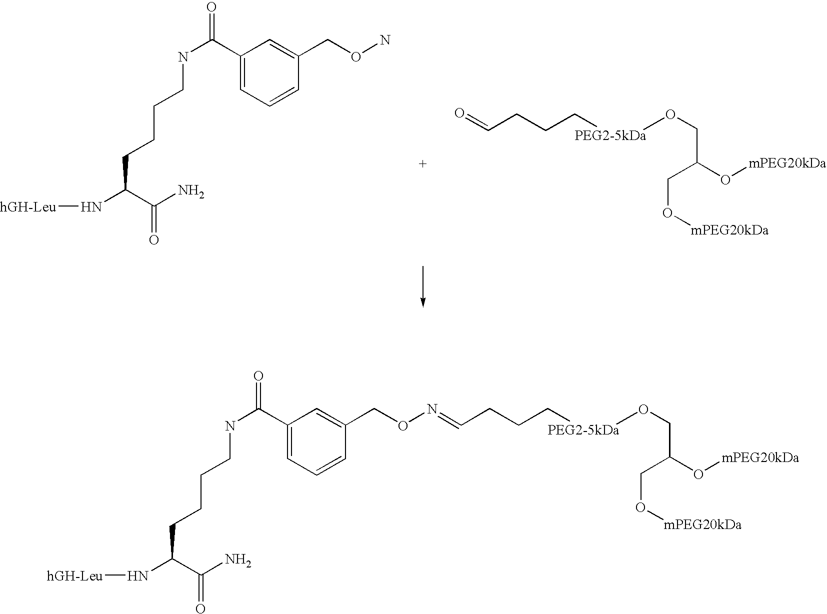

The protein solution obtained in step 1 (1.5 ml, about 10 mg/ml)) was added to the solution of “Sunbright GL3-400AL2” (NOF product) (71 mg, 1.6 μmoles, about 3 equivalents) in 0.14M 3-methylthio-1 propanol (0.5 ml).

The reaction mixture was incubated at 30° C.

After 24 h reaction time, the product was purified on ion exchange (Amersham MonoQ 10/100 GL, A buffer: Tris 50 mM pH8.5, buffer B: A+0.2M NaCl, 0 to 50% B over 20 column volume, 50 to 100% over 3 column volumes, 4 ml/min).

The pooled fractions were lyophilized after buffer shift to ammonium bicarbonate.

Yield: 4 mg of product was isolated (9% from the starting hGH-Leu-Ala protein)

### Example 18

### (S)-2-Amino-6-(3-(azidomethyl)benzoylamino)hexanoic amide

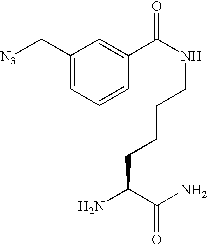

Step 1:

Pyrrolidin-2,5-dione-1-yl 3-(azidomethyl)benozoic ester

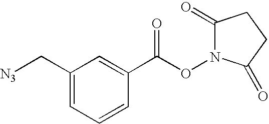

2-Succinimido-1,1,3,3-tetramethyluronium tetrafluoroborate (TSTU, 32.52 g, 107 mmol) was added to a solution of 3-(azidomethyl)benzoic acid (19.01 g, 107 mmol) and triethylamine (14.96 ml, 107 mmol) in N,N-dimethylformamide (50 ml). The reaction mixture was stirred for 16 h at room temperature. It was diluted with ethyl acetate (250 ml) and washed with water (3×120 ml). The organic layer was washed with a saturated aqueous solution of sodium hydrogencarbonate (150 ml) and dried over sodium sulphate. The solvent was removed in vacuo to give 25.22 g of pyrrolidin-2,5-dione-1-yl 3-(azidomethyl)benozoic ester.

¹H-NMR (CDCl₃) δ 2.92 (m, 4H); 4,45 (s, 2H); 7.55 (t, 1H), 7.65 (d, 2H); 8.10 (m, 2H).

Step 2:

(S)-6-(3-(Aminomethyl)benzoylamino)-2-(tert-butoxycarbonylamino)hexanoic amide

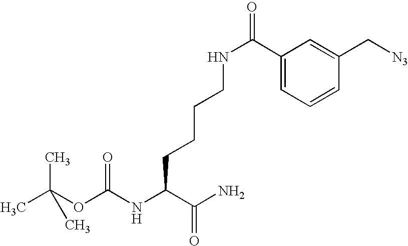

Crude (S)-5-amino-1-(carbamoyl)pentyl)carbamic acid tert-butyl ester (10.26 g, 41.82 mmol) was dissolved in N,N-dimethylformamide (150 ml). Pyrrolidin-2,5-dione-1-yl 3-(azidomethyl)benozoic ester (11.47 g, 41.822 mmol) and ethyldiisopropylamine (21.48 ml, 125.5 mmol) were added successively. The reaction mixture was stirred for 16 h at room temperature. It was diluted with ethyl acetate (500 ml) and washed first with a 10% aqueous solution of sodium hydrogensulphate (200 ml), water (3×250 ml) and a saturated aqueous solution of sodium hydrogencarbonate (200 ml). It was dried over sodium sulphate. The solvent was removed in vacuo to give 6.05 g of (S)-6-(3-(aminomethyl)benzoylamino)-2-(tertbutoxycarbonylamino)hexanoic amide.

¹H-NMR (CDCl₃) δ 1.40 (s, 9H); 1.63 (m, 4H); 1.83 (m, 2H); 3.43 (q, 2H); 4.15 (m, 1H); 4.37 (s, 2H); 5.56 (d, 1H); 6.08 (s, 1H); 6.75 (s, 1H); 7.00 (s, 1H); 7.43 (m, 2H); 7.77 (m, 2H).

MS: m/z=427 (M+Na)⁺, 305 (M-Boc)⁺.

Step 3:

Gaseous hydrogen chloride was bubbled two times for 15 min each through a suspension of (S)-6-(3-(aminomethyl)benzoylamino)-2-(tert-butoxycarbonylamino)hexanoic amide (6.05 g, 14.96 mmol) in ethyl acetate (75 ml). The solvent was removed in vacuo. The crude product was purified by 9 runs of a HPLC-chromatography on a C18-reversed phase column, using a gradient of 8-28% acetonitrile in water, which was buffered with 0.1% trifluoroacetic acid, to give together 5.03 g of the trifluoroacetic acid salt of (S)-2-amino-6-(3-(azidomethyl)benzoylamino)hexanoic amide

HPLC: 6.53 min (method O₂-b1-2).

¹H-NMR (DMSO-d₆) δ 1.36 (m, 2H); 1.55 (m, 2H); 1.75 (m, 2H); 3.26 (q, 2H); 3.70 (m, 1H); 4.53 (s, 2H); 7.52 (m, 3H); 7.84 (m, 3H); 8.06 (br, 3H); 8.54 (t, 1H).

MS: m/z=305 (M+1)⁺

### Pharmacological Methods

### Assay (I) BAF-3 GHR Assay to Determine Growth Hormone Activity

The BAF-3 cells (a murine pro-B lymphoid cell line derived from the bone marrow) was originally IL-3 dependent for growth and survival. 11-3 activates JAK-2 and STAT which are the same mediators GH is activating upon stimulation. After transfection of the human growth hormone receptor the cell line was turn into a growth hormone-dependent cell line. This clone can be used to evaluate the effect of different growth hormone samples on the survival of the BAF-3 GHR.

The BAF-3 GHR cells are grown in starvation medium (culture medium without growth hormone) for 24 hours at 37° C., 5% CO₂.

The cells are washed and re-suspended in starvation medium and seeded in plates. 10 μl of growth hormone compound or human growth hormone in different concentrations or control is added to the cells, and the plates are incubated for 68 hours at 37° C., 5% CO₂.

AlamarBlue® is added to each well and the cells are then incubated for another 4 hours. The AlamarBlue® is a redox indicator, and is reduced by reactions innate to cellular metabolism and, therefore, provides an indirect measure of viable cell number.

Finally, the metabolic activity of the cells is measure in a fluorescence plate reader. The absorbance in the samples is expressed in % of cells not stimulated with growth hormone compound or control and from the concentration-response curves the activity (amount of a compound that stimulates the cells with 50%) can be calculated.

All references, including publications, patent applications and patents, cited herein are hereby incorporated by reference to the same extent as if each reference was individually and specifically indicated to be incorporated by reference and was set forth in its entirety herein.

All headings and sub-headings are used herein for convenience only and should not be construed as limiting the invention in any way,

Any combination of the above-described elements in all possible variations thereof is encompassed by the invention unless otherwise indicated herein or otherwise clearly contradicted by context.

The terms “a” and “an” and “the” and similar referents as used in the context of describing the invention are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate).

All methods described herein can be performed in any suitable order unless otherwise indicated herein or otherwise clearly contradicted by context.

The use of any and all examples, or exemplary language (e.g., “such as”) provided herein, is intended merely to better illuminate the invention and does not pose a limitation on the scope of the invention unless otherwise indicated. No language in the specification should be construed as indicating any element is essential to the practice of the invention unless as much is explicitly stated.

The citation and incorporation of patent documents herein is done for convenience only and does not reflect any view of the validity, patentability and/or enforceability of such patent documents,

The description herein of any aspect or embodiment of the invention using terms such as “comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

This invention includes all modifications and equivalents of the subject matter recited in the aspects or claims presented herein to the maximum extent permitted by applicable law.

1. A compound according to formula I 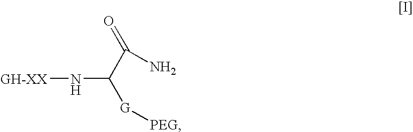
wherein GH represent a growth hormone compound; G represents R-A-E, wherein R and E each independently represents a bond or a linker, and wherein A represents a bi-radical of any chemical moiety; PEG represents a polyethylene glycol radical; and XX represent an amino acid residue selected from histidine, asparatic acid, arginine, phenylalanine, alanine, glycine, glutamine, glutamic acid, lysine, leucine, methionine, asparagine, serine, tyrosine, threonine, isoleucine, tryptophane, proline, and valine; and pharmaceutically acceptable salts, solvates and prodrugs thereof.

2. The compound according to claim 1, with a structure according 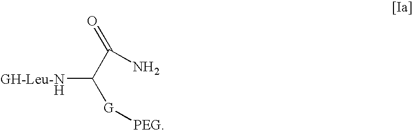
to formula Ia

3. The compound according to claim 2, with a structure according 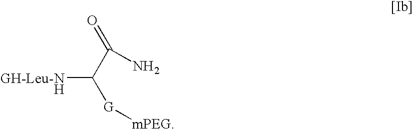
to formula Ib

4. The compound according to claim 2 with a 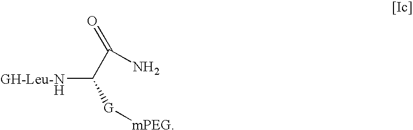
structure according to formula Ic

5. The compound according to claim 2, with a structure selected from the group consisting of: according to formula Id, Ie, or If, 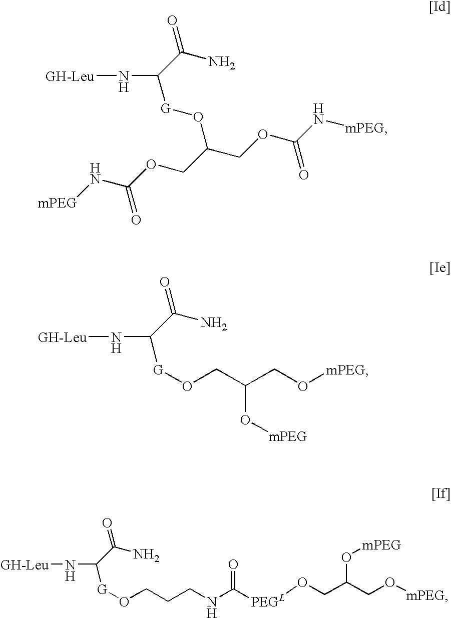
wherein PEG^{L} is a di-radical of a PEG with a molecular weight between 2 kDa and 5 kDa.

6. The compound according to claim 5 with a structure selected from the group consisting of: according to formula Ig, Ih, or Ii,

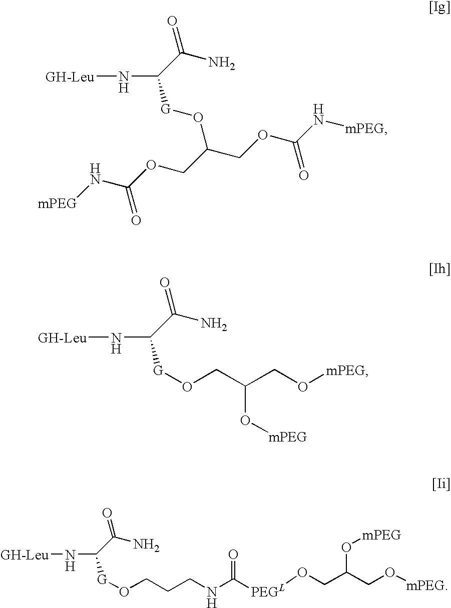

7. The compound according to claim 1, wherein GH represents a growth hormone compound comprising an amino acid sequence having at least 90% identity to the amino acid sequence of human growth hormone (hGH) (SEQ ID NO:1).

8. The compound according to claim 7, wherein GH comprises the amino acid sequence of hGH (SEQ ID NO: 1).

9. The compound according to claim 1, wherein A represents an oxime bond, hydrazone bond, phenylhydrazone bond, semicarbazone moiety, triazole bond, isooxazolidine bond, amide bond, or aralkyne bond.

10. The compound according to claim 1, wherein G is selected from

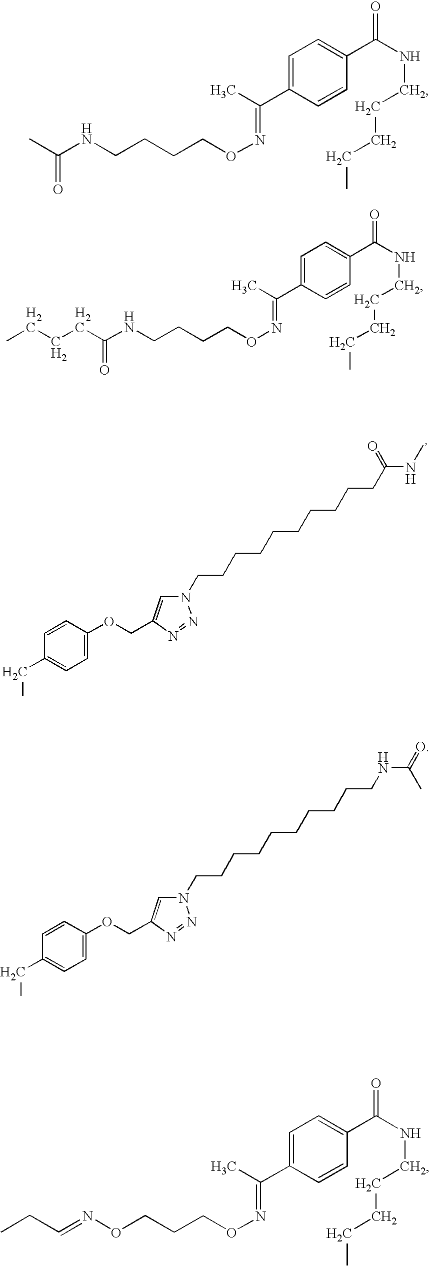

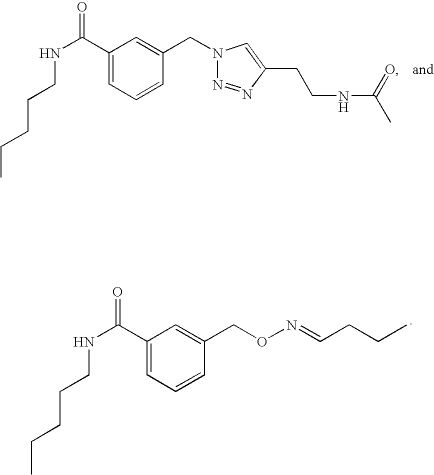

11. The compound according to claim 3, wherein mPEG represents a methoxy polyethylene glycol with a molecular weight between around 5 kDa and around 60 kDa.

12. The compound according to claim 11, wherein said mPEG represents a methoxy polyethylen glycol with a molecular weight around 10 kDa, around 20 kDa, around 30 kDa or around 40 kDa.

13. The compound according to claim 1 selected from 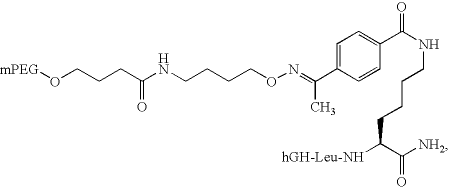
in which mPEG has a molecular weight of about 5 kDa, 10 kDa, 15 kDa, 20 kDa, 30 kDa, 40 kDa or 60 kDa; 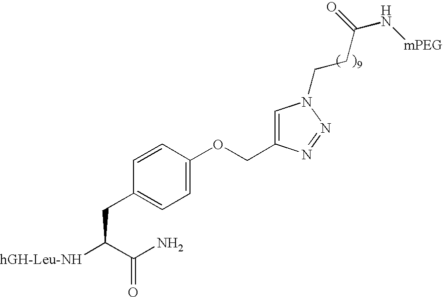
in which mPEG has a molecular weight of about 5 kDa, 10 kDa, 15 kDa, 20 kDa, 30 kDa, 40 kDa or 60 kDa; 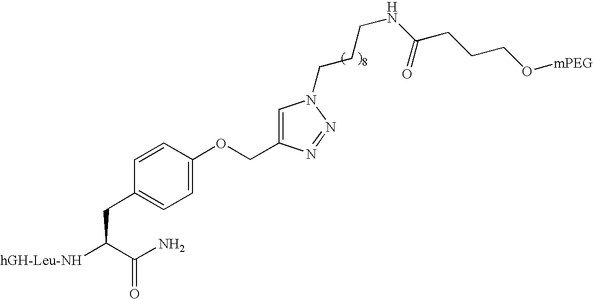
in which mPEG has a molecular weight of about 5 kDa, 10 kDa, 15 kDa, 20 kDa, 30 kDa, 40 kDa or 60 kDa; 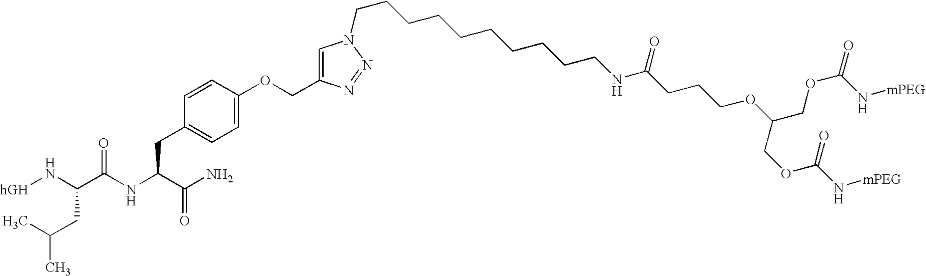
in which each mPEG has a molecular weight of about 5 kDa, 10 kDa, 15 kDa, 20 kDa, 30 kDa, 40 kDa or 60 kDa; 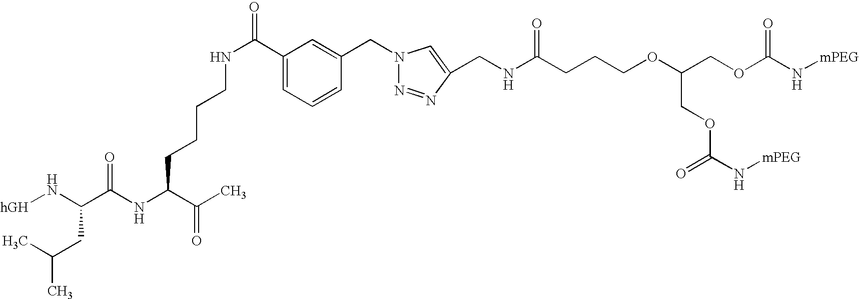
in which each mPEG has a molecular weight of about 5 kDa, 10 kDa, 15 kDa, 20 kDa, 30 kDa, 40 kDa or 60 kDa; 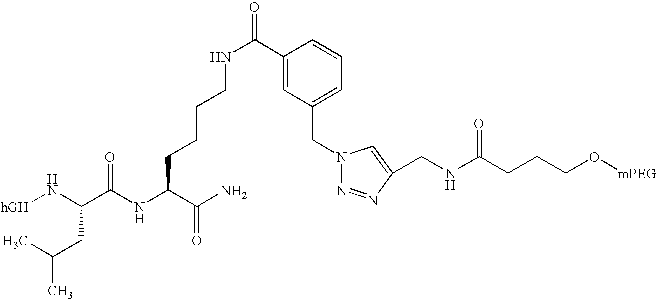
in which mPEG has a molecular weight of about 5 kDa, 10 kDa, 15 kDa, 20 kDa, 30 kDa, 40 kDa or 60 kDa; 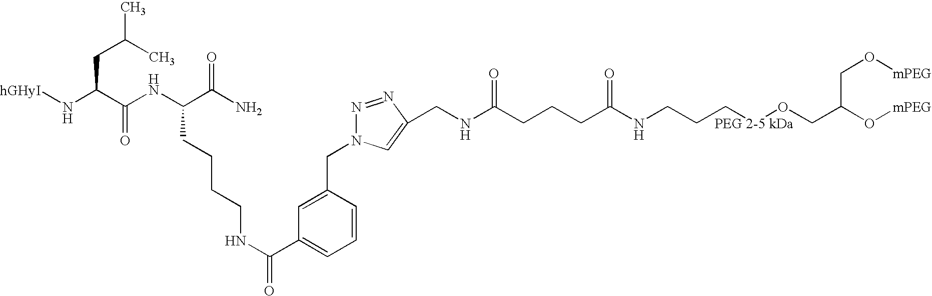
in which each mPEG has a molecular weight of about 5 kDa, 10 kDa, 15 kDa, 20 kDa, 30 kDa, 40 kDa or 60 kDa; 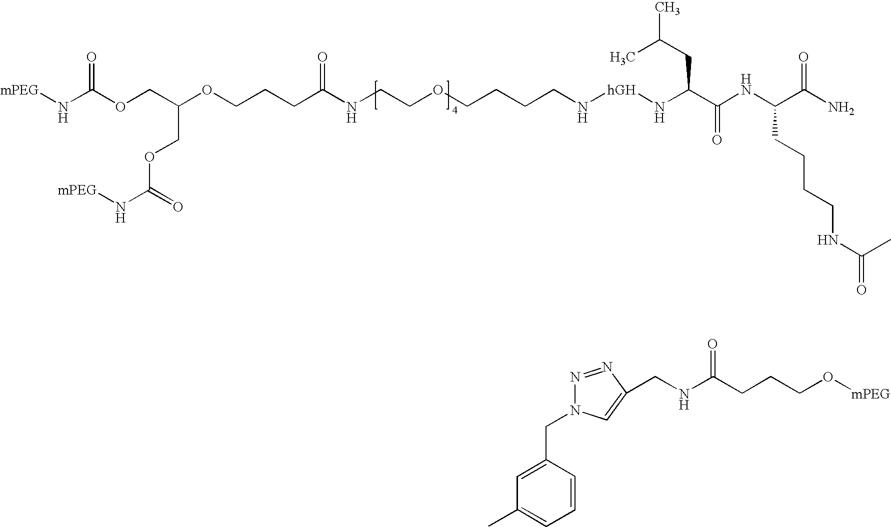
in which each mPEG has a molecular weight of about 5 kDa, 10 kDa, 15 kDa, 20 kDa, 30 kDa, 40 kDa or 60 kDa; 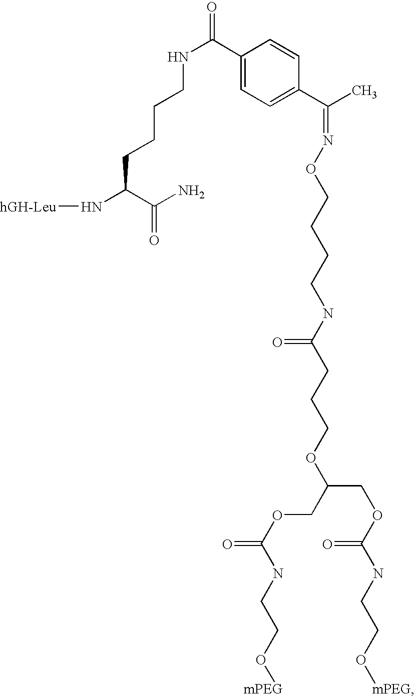
in which each mPEG has a molecular weight of about 5 kDa, 10 kDa, 15 kDa, 20 kDa, 30 kDa, 40 kDa or 60 kDa; 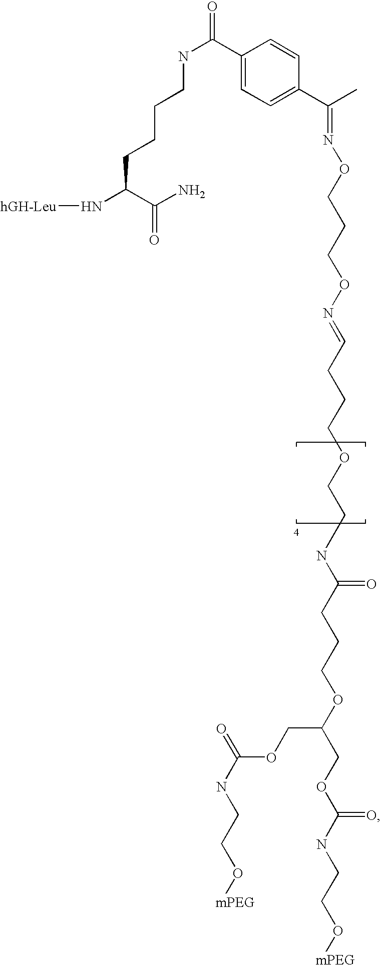
in which each mPEG has a molecular weight of about kDa, 10 kDa, 15 kDa, 20 kDa, 30 kDa, 40 kDa or 60 kDa; 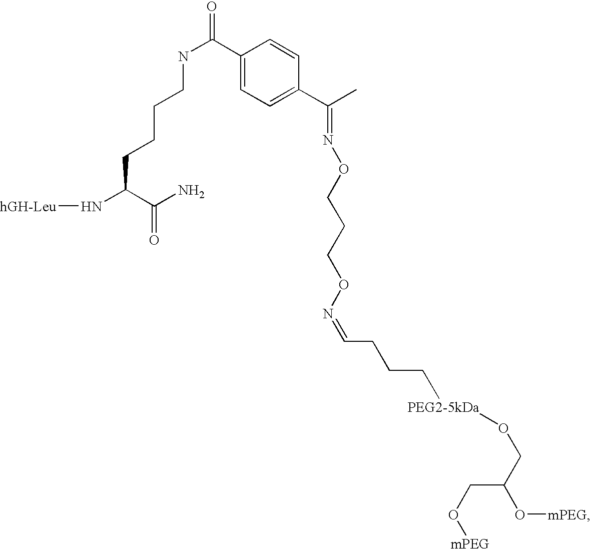
in which each mPEG has a molecular weight of about 5 kDa, 10 kDa, 15 kDa, 20 kDa, 30 kDa, 40 kDa or 60 kDa; and 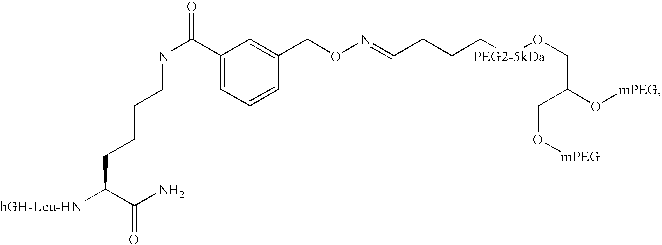
in which each mPEG has a molecular weight of about 5 kDa, 10 kDa, 15 kDa, 20 kDa, 30 kDa, 40 kDa or 60 kDa.

14. (canceled)

15. A pharmaceutical composition comprising a compound according to formula I 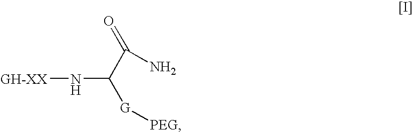
wherein GH represent a growth hormone compound; G represents R-A-E, wherein R and E each independently represents a bond or a linker and wherein A represents a bi-radical of any chemical moiety; PEG represents a polyethylene glycol radical; and XX represent an amino acid residue selected from histidine, asparatic acid, arginine, phenylalanine alanine, glycine, glutamine, glutamic acid, lysine, leucine, methionine, asparagine, serine tyrosine, threonine, isoleucine tryptophane, proline and valine; and pharmaceutically acceptable salts solvates and prodrugs thereof, optionally in combination with a pharmaceutical excipient.

16. A method of treating growth hormone deficiency (GHD), the method comprising administrating to a patient in need thereof an effective amount of a therapeutically effective amount of a compound according to formula I 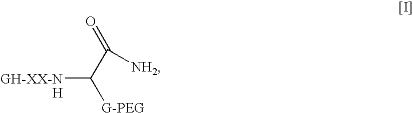
wherein GH represent a growth hormone compound; G represents R-A-EB wherein R and E each independently represents a bond or a linker and wherein A represents a bi-radical of any chemical moiety; PEG represents a polyethylene glycol radical; and XX represent an amino acid residue selected from histidine, asparatic acid, arginine, phenylalanine alanine, glycine, glutamine, glutamic acid, lysine, leucine, methionine, asparagine, serine tyrosine, threonine, isoleucine tryptophane, proline and valine; and pharmaceutically acceptable salts solvates and prodrugs thereof.

17. A method of treating Turner Syndrome; Prader-Willi syndrome (PWS); Noonan syndrome; Down syndrome; chronic renal disease, juvenile rheumatoid arthritis; cystic fibrosis, HIV-infection in children receiving HAART treatment (HIV/HALS children); short children born short for gestational age (SGA);
short stature in children born with very low birth weight (VLBW) but SGA; skeletal dysplasia; hypochondroplasia; achondroplasia; idiopathic short stature (ISS); GHD in adults; fractures in or of long bones, such as tibia, fibula, femur, humerus, radius, ulna, clavicula, matacarpea, matatarsea, and digit; fractures in or of spongious bones, such as the scull, base of hand, and base of food; patients after tendon or ligament surgery in e.g. hand, knee, or shoulder; patients having or going through distraction oteogenesis;
patients after hip or discus replacement, meniscus repair, spinal fusions or prosthesis fixation, such as in the knee, hip, shoulder, elbow, wrist or jaw; patients into which osteosynthesis material, such as nails, screws and plates, have been fixed; patients with non-union or mal-union of fractures; patients after osteatomia, e.g. from tibia or 1^{st} toe; patients after graft implantation; articular cartilage degeneration in knee caused by trauma or arthritis; osteoporosis in patients with Turner syndrome; osteoporosis in men; adult patients in chronic dialysis (APCD); malnutritional associated cardiovascular disease in APCD; reversal of cachexia in APCD; cancer in APCD; chronic abstractive pulmonal disease in APCD; HIV in APCD; elderly with APCD; chronic liver disease in APCD, fatigue syndrome in APCD; Crohn's disease; impaired liver function; males with HIV infections; short bowel syndrome; central obesity; HIV-associated lipodystrophy syndrome (HALS); male infertility; patients after major elective surgery, alcohol/drug detoxification or neurological trauma; aging; frail elderly; osteo-arthritis; traumatically damaged cartilage; erectile dysfunction; fibromyalgia; memory disorders; depression; traumatic brain injury; subarachnoid haemorrhage; very low birth weight; metabolic syndrome; glucocorticoid myopathy; short stature due to glucucorticoid treatment in children, the acceleration of the healing of muscle tissue, nervous tissue or wounds; the acceleration or improvement of blood flow to damaged tissue; or the decrease of infection rate in damaged tissue, the method comprising administrating to a patient in need thereof an effective amount of a therapeutically effective amount of a compound according to formula I 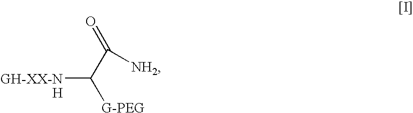
wherein GH represent a growth hormone compound; G represents R-A-E, wherein R and E each independently represents a bond or a linker and wherein A represents a bi-radical of any chemical moiety PEG represents a polyethylene glycol radical; and XX represent an amino acid residue selected from histidine, asparatic acid, arginine, phenylalanine alanine, glycine, glutamine, glutamic acid, lysine, leucine, methionine, asparagine, serine tyrosine, threonine, isoleucine tryptophane, proline and valine; and pharmaceutically acceptable salts solvates and prodrugs thereof.

18.-19. (canceled)

20. A method for the preparation of a compound according to formula I 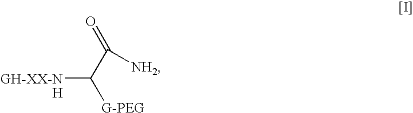
wherein GH represent a growth hormone compound; G represents R-A-EB wherein R and E each independently represents a bond or a linker and wherein A represents a bi-radical of any chemical moiety PEG represents a polyethylene glycol radical; and XX represent an amino acid residue selected from histidine, asparatic acid, arginine, phenylalanine alanine, glycine, glutamine, glutamic acid, lysine, leucine, methionine, asparagine, serine tyrosine, threonine, isoleucine tryptophane, proline and valine; and pharmaceutically acceptable salts solvates and prodrugs thereof, the method comprising the steps of reacting GH-XX-Ala in one or more steps with a first compound, which is an α-amino acid amide represented by the formula 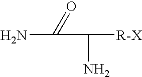
in the presence of Carboxypeptidase Y (CPY) to form a transacylated compound of the formula 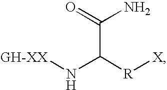
said transacylated peptide being further reacted in one or more steps with a second compound of the formula
Y-E-PEG
to form a conjugated GH of the formula 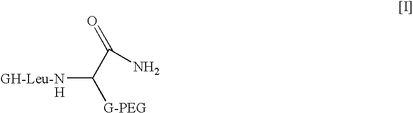
wherein G represent R-A-E, wherein R represents a linker or a bond; E represents a linker or a bond; A represents the moiety formed by the reaction between the functional groups comprised in X and Y; and
GH represent a growth hormone compound;
X represents a radical comprising a functional group not accessible in the amino acid residues constituting the GH;
Y represents a radical comprising one or more functional groups which groups react with functional groups present in X, and which functional groups do not react with functional groups accessible in the GH;
PEG represent a poly ethylene glycol moiety;
and XX represents an amino acid residue selected from histidine, asparatic acid, arginine, phenylalanine, alanine, glycine, glutamine, glutamic acid, lysine, leucine, methionine, asparagine, serine, tyrosine, threonine, isoleucine, tryptophane, proline, and valine.

21. The method according to claim 20, wherein XX represents Leu.

22. The method according to claim 20, wherein said α-amino acid amide is selected from 2-amino-3-oxo-butyramide, 2-amino-6-(4-oxo-pentanoylamino)-hexanoic acid amide, 2-amino-3-(2-oxo-2-phenylethylsulfanyl)-propionamide, 2-amino-5-oxo-hexanoic acid amide, 2-amino-3-oxopropionamide, 2-amino-6-(4-acetylbenzoylamino)hexanoic acid amide, (2S)-2-amino-3-[4-(2-oxopropoxy)phenyl]propionamide, (2S)-2-amino-3-[4-(2-oxobutoxy)phenyl]propionamide, (2S)-2-amino-3-[4-(2-oxopentoxy)phenyl]propionamide, (2S)-2-amino-3-[4-(4-oxopentoxy)phenyl]propionamide, (2S)-2-amino-6-(4-oxo-4-phenylbutyrylamino)hexanoic acid amide, (2S)-2-amino-6-(4-oxo-4-(4-chlorophenylbutyrylamino)hexanoic acid amide, 3-acetyl-N-((5S)-5-amino-5-carbamoylpentyl)benzamide, 2-acetyl-N-((5S)-5-amino-5-carbamoylpentyl)benzamide, (2S)-2-amino-3-(4-(prop-2-ynyloxy)phenyl)propionamide, (S)-2-aminopent-4-ynoicacid amide, (2S)-2-amino-6-(3-(prop-2-ynyl)benzoylamino)hexanoic amide, (2S)-2-amino-6-(4-(prop-2-ynyl)benzoylamino)hexanoic amide, (2S)-2-amino-6-(2-(prop-2-ynyl)benzoylamino)hexanoic amide, (2S)-2-amino-3-[4-(1-oxoethyoxy)phenyl)propionamide,(S)-2-amino-6-(3-(aziodmethyl)benzoylamino)hexanoic amide, (S)-2-amino-6-(3-(aminoxymethyl)benzoylamino)hexanoic amide, and S-phenylacylcysteine amide.

23. The method according to claim 20, wherein Y-E-PEG represents 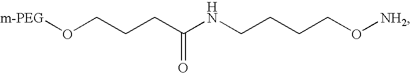
wherein mPEG has a molecular weight of 20 kDa or 30 kDa, 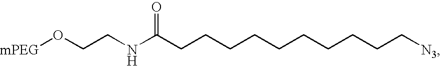
wherein mPEG has a molecular weight of 20 kDa or 30 kDa, 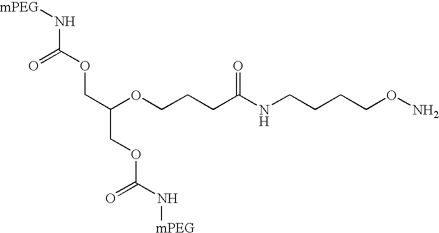
wherein mPEG has a molecular weight of 20 kDa or 30 kDa, 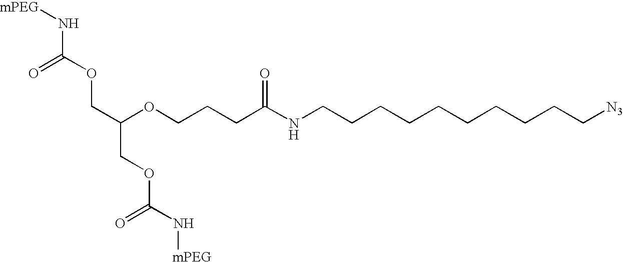
wherein mPEG has a molecular weight of 20 kDa or 30 kDa, 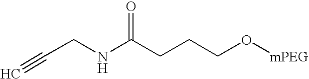
wherein mPEG has a molecular weight of 20 kDa or 30 kDa, 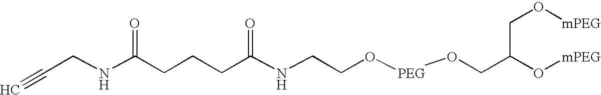
wherein mPEG has a molecular weight of 20 kDa or 30 kDa, and PEG has a molecular weight between 2 and 5 kDa, 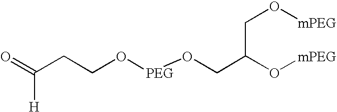
wherein mPEG has a molecular weight of 20 kDa or 30 kDa, and PEG has a molecular weight between 2 and 5 kDa, or 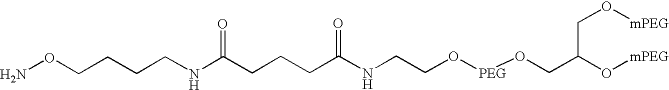
wherein mPEG has a molecular weight of 20 kDa or 30 kDa and PEG has a molecular weight between 2 and 5 kDa.

24. A compound of the formula GH-XX-Ala, wherein GH comprises a peptide of an amino acid sequence having at least 90% identity to the amino acid sequence of hGH (SEQ ID NO:1), and XX represents an amino acid residue selected from histidine, asparatic acid, arginine, phenylalanine, alanine, glycine, glutamine, glutamic acid, lysine, leucine, methionine, asparagine, serine, tyrosine, threonine, isoleucine, tryptophane, proline, and valine.

25. The compound according to claim 24, wherein GH is hGH.

26. The compound according to claim 25, which is hGH-Leu-Ala.
